(19) United States
(12) Patent Application Publication
(10) Pub. No.: US 2017/0370917 A1
(43) Pub. Date: Dec. 28, 2017

(54) MOLECULAR SENSORS

(71) Applicant: UNIVERSITY COLLEGE DUBLIN, NATIONAL UNIVERSITY OF IRELAND, DUBLIN, Dublin (IE)

(72) Inventors: Donal Mackernan, Dublin (IE); Shourjya Sanyal, Dublin (IE)

(21) Appl. No.: 15/694,592

(22) Filed: Sep. 1, 2017

Related U.S. Application Data
(63) Continuation-in-part of application No. PCT/IB2016/051249, filed on Mar. 4, 2016.

(30) Foreign Application Priority Data
Mar. 4, 2015 (GB) 1503671.8
Apr. 30, 2015 (GB) 1507378.6
Oct. 15, 2015 (GB) 1518231.4

(51) Int. Cl. G01N 33/542; C07K 14/47; G01N 33/58; G01N 21/64; C12N 9/12; C12N 9/10; A61K 38/00

(52) U.S. Cl. CPC G01N 33/542; C12N 9/12; C07K 14/47; C12N 9/1077; C12Y 204/02036; G01N 33/582; G01N 21/6428; C12Y 207/11011; C07K 2319/55; A61K 38/00; C07K 2319/61; G01N 2021/6439

(57) ABSTRACT

The invention relates to a sensor molecule for detecting a target molecule comprising: (a) a rod-like molecule L and a rod-like molecule R connected to each other by a joint molecule C to form a hinge; (b) a target binding molecule A bonded to the end of rod-like molecule L opposite to the joint molecule C; (c) a binding molecule A′ bonded to the end of rod-like molecule R opposite the joint molecule C; wherein the target binding molecule A is arranged to bind to the target molecule to be detected, and binding molecule A′ is arranged to bind to: i) the same target molecule as target binding molecule A; or ii) a complex of the target binding molecule A and the target; and wherein the hinge is biased into an open position, such that target binding molecule A and binding molecule A′ are biased apart by the hinge.

### CROSS-REFERENCING

This application is a continuation-in-part of International Application No. PCT/IB2016/051249, filed on Mar. 4, 2016, which claims benefit of priority to United Kingdom Application Nos. 1503671.8, filed on Mar. 4, 2015, 1507378.6, filed on Apr. 30, 2015, and 1518231.4, filed on Oct. 15, 2015, which applications are incorporated by reference herein.

### TECHNICAL FIELD

The invention relates generally to the field of molecular biology, food and health science and metabolomics. More specifically, the invention relates to molecular sensors, such as unimolecular sensors, and methods using intramolecular resonance energy transfer (RET) and fluorescence for detecting the presence of analytes or ligand binding in assays and in vivo, including the use of immunoassays to detect analytes such as antigens and antibodies in the life and environmental sciences and related industries. The invention also relates to analyte dependent activation of pharmaceuticals and chemo-toxins.

### BACKGROUND

A variety of genetic biosensors have been developed since the discovery of green fluorescent protein (FP) and its subsequent cloning from the jellyfish Aequorea Victoria. These biosensors have permitted the investigation of cellular mechanisms using optical microscopy, permitting the tracking of analytes (i.e. cellular proteins and other signalling molecules) within their endogenous environment (as described for example in reference NP1), and in vitro. The FP's when built into appropriate sensors can effectively illuminate the internal workings of the cell. As the sensors are proteins, they can be inserted within the cellular compartments to report on local concentrations of analytes or ligands of interest, which is known as genetic targeting, through “off the shelf” biotechnology kits, provided appropriate sequences of amino-acid residues have been determined, or designed. It is this design step which arguably now has become the most demanding and rewarding, and created, for instance, opportunities to investigate signalling networks in living cells. The range of application of such biosensors includes use as neurotransmitter sensors, in high-throughput screening drug discovery, the observation of turnover of select metabolites at the single cell level in real time, and the visualization of specific macromolecular machines within the cellular environment.

Biosensors can be designed to respond to changes in local analyte concentrations, by changing their internal structures, which in turn can be observed optically, through changes in the fluorescence properties of individual FP's, or changes in the resonance energy transfer (RET) rates between pairs of FP's, termed the donor and acceptor. Sensors designed to exploit RET are particularly capable of providing fine scale spatial and temporal information within cellular compartments. In some cases the light source for these probes is endogenous, coming from naturally bio-luminescent proteins, so called BRET probes. However, most require an external light source, and are termed FRET probes (described generally in references NP2, NP3, NP4).

In each case, the quantum mechanism used is the same. If the donor is in a suitable optically excited state, and if the distance r between it and the other chromophore (the acceptor) is sufficiently close (1-10 nm) (see reference NP5) RET can take place. As RET efficiency typically has an r⁻⁶ dependence, changes in r can effectively switch RET on and off. It is the nature and design of this molecular and genetically targetable switch that lies at the heart of the success of FRET based microscopy in biology.

The following six representative examples of genetic biosensors devised to monitor/report on local intracellular concentrations serve to explain the context of the present invention, and in particular the problem it addresses and solves.

The first example is provided by the biosensor cameleon-1 (see reference NP6), which was the first genetically encodable Ca²⁺ indicator. It was created by placing a protein composed of the C-terminus of calmodulin (CaM) and the CaM binding peptide M13, between the FRET pair Cyan Fluoroscent Protein (CFP) and Green Fluroscent Protein (GFP). Changes in the intracellular concentration of the indicator induce conformational changes between CAM and M13, corresponding changes in the distance and relative orientation between CFP and GFP, and ensuing variation in the FRET rate. Through various mutations, derivatives of cameleon-1 can now measure Ca²⁺ molar concentrations ranging from 10⁻⁷ to 10⁻⁴ M in a variety of cellular compartments.

The second example is provided by Allen and Zhang (see references NP7, NP8), who, motivated by a desire to track cAMP activity, for example at the inner side of the plasma membrane, cytoplasm, nucleus, and mitochondria in living cells, systematically optimized probes through circular permutation (cp).

The third example is provided by Lissandron et al (see reference NP9) who improved cAMP unimolecular probes combining molecular simulation with experiment to optimise linkers through mutation of selective residues, and thereby almost doubled FRET efficiency and substantially improved “dynamic range” (a measure of the signal to noise ratio).

The fourth example is provided by Pertz et al. (see reference NP10), who by focusing on the Rho family of GTPases (G proteins) which regulate the actin and adhesion dynamics that control cell migration, developed a fluorescent biosensor to visualize the spatiotemporal dynamics of RhoA activity during cell migration, and subsequently engineered a library of probes (see reference NP4) by varying several geometrical parameters, such as fluorophore distance (using linkers of different length), dipole orientation (using cp mutants in both the acceptor and donor fluorophores), and sensing module domain topology.

Despite the widespread use and development of RET reporters such as those mentioned above, various difficulties remain. For instance, a strong RET signal, and a strong signal to noise ratio, can be achieved by increasing the tendency of the FP's to bind in the ON state (i.e., when the analyte binds to the ligand binding domain as a result of its intracellular concentration going beyond an effective threshold value.) But this tends also to increase the perturbation of the endogenous system, making it more difficult to track fine scale changes in time and space. For instance, the over-expression of the biosensors designed to measure analytes such as Ca²⁺ can interfere with the proper function of endogenous CaM molecules. As another illustration of the difficulty, consider the demands made of a probe to monitor protein kinases activity which are often dynamically regulated. It is desirable that the probe can continuously track up and down regulation of kinase activities whilst maintaining a strong RET signal and a high signal to noise ratio, yet is difficult to realise in practice, as pointed out by a fifth set of examples of the (see references NP1, NP11.)

A sixth example of over-expression of bio-sensors is found in the work of Palmer et al., who employed a “bump and hole” strategy to diminish interactions between wild-type CaM and the M13 peptide in the sensor, while maintaining the sensitivity of the reporter (see reference NP12). But despite their success in overcoming such difficulties for a particular reporter, an approach and associated general purpose mechanism applicable to a large and quite wide class of reporters has remained elusive.

In effect the ligand binding domain and sensor domain remain in the ON state long after they should have separated if they were to be able to respond sensitively to up and down variations of the analyte concentration corresponding to the endogenous system. As a consequence, the FRET signal remains high long after it should desirably have dropped.

Immunoassays are biochemical tests used to measure the presence and concentration of analytes using antibodies or immunoglobulin. A wide variety of macromolecules can be detected, including antigens, and antibodies. In general, the detection of the analyte involves one or more antibodies binding to it, with at least one antibody being “labelled” with some form of marker molecule, frequently a fluorescent protein, a dye, or an enzyme. For instance, the enzyme-linked immunosorbent assay (ELISA) is a test that is extensively used as a diagnostic tool in wide variety of applications from medicine and plant pathology to environmental science and food industry. In the simplest version of this test, known as Direct ELISA, a microtiter surface is exposed to an antigen solution for enough time that the antigen bonds to the surface, to which subsequently is added a primary antibody conjugated with an enzyme. After some time, a substrate is also added to the microtiter, which reacts with the enzyme, thereby changing colour. The microtiter is then washed to remove the excess—leaving the effectively stained antibodies bound to the plastic surface. Indirect ELISA is similar to Direct ELISA, except that the primary antibody is not conjugated. Instead an additional secondary antibody conjugated with an enzyme is added, which binds to the primary antibody. In yet another assay known as Sandwich ELISA, a primary antibody is anchored to a microtiter surface, to which is added a solution containing the antigen, and another primary antibody capable of binding to a different epitope on the antigen. The excess antibody is washed away, and another solution containing an enzyme conjugated secondary antibody which binds to the primary antibodies. In sandwich ELISA, the presence of the antigen effectively turns on an attractive indirect interaction between the two primary antibodies. In competitive ELISA, like Direct ELISA, the unlabelled primary antigen is incubated with the antigen in a microtiter so that it binds to the surface. In the next step, a solution including unconjugated antigens and primary antibodies is added to microtiter, and incubated. The surface is then washed to remove unbound antigens and antibody, and then an enzyme conjugated secondary antibody is added which binds to the primary antibody-antigen complex. The key feature of this technique is the competition between the antigens attached to the microtiter surface and antigens in solution to bind to primary antibodies. The above ELISA tests can be used to detect either antigens, or antibodies, and are described in detail in reference NP28.

Immunofluorescence is an alternative method to measure the presence of antigens, where the role of the enzyme in ELISA tests is instead played by fluorescent proteins or dyes. Thus, in Direct immunofluorescence, a fluorescent molecule or dye is conjugated to the primary antibody, which can be viewed through a microscope. In Indirect, Sandwich and Competitive immunofluorescence, a fluorescent molecule or dye is conjugated to the secondary antibody, which can be viewed through a microscope. As in ELISA, many washing and incubating steps are required in the above immunofluorescence assays, and are described in detail in reference NP29.

Western Blot is another form of immunoassay where the target analytes are proteins, and which combines immunofluorescence with gel electrophoresis to more easily identify different analytes according to their molecular weight. NP30.

To reduce the problem of unintended cross interactions between antibodies and other immune-interactions, it is also possible to use specific fragments of antibodies such as IgG and IgM, capable of binding to one or more epitopes on the antigen, such as F(ab′)2, Fab, Fab′ and Fv as described in references NP31 and NP32.

In both ELISA based assays and immunofluorescence, it is necessary to conjugate antibodies with appropriate labels, such as dyes (Cyanine Dyes, Fluoroscine, Rhodamine, Texas red, Aminomethylcoumarin and Phycoetherine), enzymes or fluorescent proteins. Other methods such as split GFP (or split Gaussia Luciferase) can also be used for tagging, where the two moieties fluoresce (or bioluminesce) only when they are brought together to form again a functional GFP (or bioluminescent molecule). Further details are given, for instance, described in references NP33 and NP34.

These conjugation methods target specific chemical groups available in the antibodies including tyrosine, lysine, glutamate, aspartate, methionine, serine, histidine, and arginine. The most common chemical reactions used target primary amines (—NH2), carboxyls (—CHO) and thiolates (SH). Lysine, which contains a primary amine, is a very common residue on practically all antibodies, and is the primary targeting site for conjugation. However this conjugation method can on occasion reduce the ability of the antibody to recognise corresponding antigen(s) due to accidental unintended labelling at the F(ab) region of the antibody. For this, and other reasons, carboxyls are perhaps the second most common labelling target, where typically the (—CHO) conjugation sites are on the Fc region of the antibody, without significantly affecting the antigen-binding capacity. In the case of antibody fragments (e.g. F(ab)₂, Fc, Fv), thiolates are the typical choice of labelling target.

The standard methods and reagents used for conjugation are described in references NP31 and NP35, and for the specific case of suflhydryls, see also references NP36 and NP33. The standard chemistry for labelling of amines uses either heterobifunctional reagents, or NHS esters, or carbodiimides, or sodium periodate. In the case of carboxyls, the carbohydrates must first be oxidized to create reactive aldehydes. In the case of carboxyls, the carbohydrates must first be oxidized to create reactive aldehydes. If primary amines are accessible on the label, the reacted aldehyde in the carbohydrates can be conjugated using reductive amination. In the absence of accessible primary amines on the label, the reacted aldehyde in the carbohydrates can be conjugated using hydrazide groups. For example, if the label is a protein, the hyrazide group can be functionalised selectively on either its C or N terminals. In the case that two different types of antibodies (for example a primary and a secondary antibody) are to be bound to the C and N terminals of a protein respectively, a blocked hyrazide group can be added to the C terminal of the protein, which can be subsequently chemically unblocked after conjugation of the first antibody at the N terminal of the protein (or vice-versa). Conjugation at sulphur atoms requires that the thiols exist as free suflhydryls (—S) (using for instance reagents such as DTT and TCEP), which can be reacted to the label activated with maleimide or iodoacetyl groups.

Although the assay methods described above are widely used, they have a number of difficulties. Typically they require several washing steps to remove excess antibodies which have not bonded to their target antigens or primary antibodies, and as a consequence are laborious or require specialised, bulky and expensive equipment. Estimating the actual concentration of the analyte(s) present in a sample is generally difficult, indirect and not accurate. Measurements cannot easily be made to track real time changes in the concentration of analytes, other than by taking consecutive samples and running each sample through complex protocols of washing, and addition of various reagents etc.

The microtiter described above can be of various shapes and sizes, including Micro and Macro arrays, Micro-well arrays, Micro-zone arrays fabricated in paper and Microfluidic chips (described generally in references NP37, NP38 and NP39).

Binding in these assays is frequently detected using, for instance, confocal scanning microscopy, and more recently with desktop scanners. Confocal microscopy uses a laser scanner (described in reference PT2) and a microscope to build up a three dimensional image of a sample through a series of two dimensional images of the sample made at various depths. In the context of immunoassays, the corresponding images can be at a cellular or sub-cellular level. The main drawback of this complex and rather fragile machinery is cost. Desktop scanning cannot be easily used to make three dimensional images. However for surface imagery, it is much cheaper than confocal microscopy, largely because the laser and complex optical lenses and mirrors used in the latter are replaced by a either a single LED of a single wavelength or a combination of Red-Green-Blue LEDs producing the target wavelength, and an ordinary CCD camera. Another method of measuring immunofluorescence uses single or multiple LED's as combined with photomultiplier diode chips.

Chromophores including fluorescent proteins can be combined with quenching nanoparticles such as gold (described in reference NP41) and antibodies to detect analytes, and to heat tissue in living samples (described in reference NP42).

As an alternative to localised tissue heat treatment, photodynamic therapy uses photosensitizer or photosensitizing agents such as porphyrin and phthalocyanin, to expose, for instance cancerous cells to reactive oxygen species upon exposure of the photosensitizing agent to electro-magnetic fields/light of appropriate wavelengths (described in reference NP43). Photodynamic therapy can also be used to treat microbial infection (described in reference NP44). It is also possible to combine photosensitizing agents with antibodies targeting specific antigens, and even to form larger complexes such as antibody-phthalocyanine-gold nanoparticle conjugates.

An aim of the present invention is to provide an improved molecular sensor that is capable of overcoming some of the problems of the above described sensors and methods.

According to a first aspect of the invention, there is provided a sensor molecule for detecting a target molecule comprising:
(a) a rod-like molecule L and a rod-like molecule R connected to each other by a joint molecule C to form a hinge;
(b) a target binding molecule A bonded to the end of rod-like molecule L opposite to the joint molecule C;
(c) a binding molecule A′ bonded to the end of rod-like molecule R opposite the joint molecule C;
   wherein the target binding molecule A is arranged to bind to the target molecule to be detected, and binding molecule A′ is arranged to bind to:
i) the same target molecule as target binding molecule A; or
ii) a complex of the target binding molecule A and the target; and
wherein the hinge is biased into an open position, such that target binding molecule A and binding molecule A′ are biased apart by the hinge.

In one embodiment, the presence and binding of a target molecule by target binding molecule A, and the binding of binding molecule A′ to either i) the target molecule or ii) the complex of the target binding molecule and target molecule, is arranged to bias the hinge into a closed position in opposition to the force of the hinge, which is biased to an open position.

The sensor molecule may comprise a state denoted as the ON state wherein A is attracted towards A′ and the hinge is arranged to repeatedly open and close. For example, the hinge will close due to A and A′ being brought into closer proximity by binding to each other in the presence of the target molecule, or both binding to the target molecule, by temporally overcoming the bias energy of the hinge to open. The bias energy of the hinge to open can temporarily force A and A′ apart. Therefore, the sensor molecule is in a state of dynamic switching to an open and closed position in the presence and detection of a target molecule. This dynamic state is designated the ON state.

The sensor molecule may comprise a state denoted as the OFF state wherein the hinge is in an open position and A is not attracted towards A′.

The ON and/or OFF state may be detectable. Additionally or alternatively, a transition between the ON and OFF states may be detectable. In one embodiment, the ON state is detectable.

In one embodiment, the binding molecule A′ is not capable of binding (or not arranged to bind) to the target binding molecule A in the absence of the target molecule (i.e. not complexed with the target molecule). For example, in one embodiment the binding molecule A′ does not have affinity for the target binding molecule A in the absence of the target molecule. The lack of affinity of the binding molecule A′ for the target binding molecule A alone (i.e. not complexed with the target molecule) prevents the sensor molecule from inadvertently switching to the ON state in the absence of the target.

The binding of target binding molecule A to the target molecule may be direct binding or through one or more intermediate molecules. Additionally, the binding of binding molecule A′ to the target molecule may be direct binding or through one or more intermediate molecules.

A characteristic feature of the present invention is that it solves the problem of the prior art described above (of the ligand binding domain and sensor domain remaining in the ON state long after they should have separated if they were to be able to respond sensitively to up and down variations of the analyte concentration corresponding to the endogenous system) in a counter-intuitive way by not requiring the ligand binding domain and sensor domain to be tightly bound to each other in the ON state. Instead the ON state is characterised by frequent transitions from a bound conformation (where the ligand binding domain and sensor domain are essentially in contact) to an unbound conformation (where the ligand binding domain and sensor domain are far from being in contact) and vice-versa. The OFF state is essentially always unbound.

In one embodiment, the target binding molecule A and/or the binding molecule A′ are capable of emitting a signal for detection when they are in proximity to each other, or bound to each other. Alternatively, the sensor may further comprise a signal molecule B and a signal molecule B′.

The detectable ON state signal may be provided by the pair of signal molecules B and B′ being brought into sufficient proximity to cause a detectable ON state signal to be emitted.

The signal molecule B and/or B′ may comprise a chromophore, fluorophore or bioluminescent molecule. In another embodiment, the target binding molecule A and/or binding molecule A′ may comprise a chromophore, fluorophore or bioluminescent molecule. The fluorophores or bioluminescent molecules may be photo-activatable (such as PA-mRFP1 or PA-mCherryl), photo-convertible convertible (such as Kaede or Dendra2), photo-switchable (such as Dronpa or Pardon), fluorescent protein timers (such as DsRed-E5 or Fast-FT), or phosphorescent. One example is to use the reversible photoswitching of Dronpa mutant K145N (see Zhou et. al. Optical Control of Protein Activity by Fluorescent Protein Domains. Science. 2012 Nov. 9; 338(6108): 810-814) and thereby the light dependent association-disassociation could be used to trigger the ON and OFF state of the biosensor. When used in association with a sensor and ligand binding domain these can be used to get a better signal to noise, as well as measure the binding energy.

In one embodiment, the detectable ON state signal is provided by resonance energy transfer (RET) between signal molecule B and signal molecule B′. In another embodiment, the detectable ON state signal is provided by resonance energy transfer (RET) between target binding molecule A and binding molecule A′. Signal molecules B and B′ may undergo measurable resonance energy transfer when sufficiently close to each other when the sensor molecule is in the ON state (e.g. when A′ and A are closer together). The resonance energy transfer (RET) may be Förster resonance energy transfer (FRET) or bioluminescent resonance energy transfer (BRET).

The signal molecule B may be bound to ligand binding molecule A and the signal molecule B′ may be bound to binding molecule A′ (or vice versa). Alternatively, the signal molecule B may be bound to the end of rod-like molecule L opposite the joint molecule C and the signal molecule B′ may be bound to the end of the rod-like molecule R opposite the joint molecule C (or vice versa). In embodiments wherein B and B′ are respectively bound to A and A′ (or vice versa), the binding may be direct, or via a spacer molecule. The binding may be covalent.

In one embodiment sensor molecules B and B′ each comprise a part of a split molecule. A split molecule may be a functioning molecule that can be split into two or more parts to a non-functioning state, and can be rejoined when the two or more parts are brought back together, such that the function is restored. For example, when brought close together due to the close presence of the target molecule, the split molecule parts may undergo resonance energy transfer in the presence of a suitable electro-magnetic field of external or endogenous origin. In one embodiment the split molecule is a bioluminescent molecule, which is capable of being split into parts, whereby the re-joining (or at least the close proximity) of the parts can lead to a restored bioluminescent function.

The split molecule may comprise a split fluorescent protein. Examples of split fluorescent proteins are well known in the art. For example the split fluorescent protein may comprise green fluorescent protein (GFP). A cleavage/split site of GFP is known to be between strand 10 and 11 of GFP. In another embodiment, the split fluorescent protein may comprise yellow fluorescent protein (YFP) or cyan fluorescent protein (CFP). Examples are described in US 20120282643 A1.

An advantage of the sensor molecule of the invention is that it makes the use of split FP's or split bioluminescent proteins reversible. Previously in the art, irreversibility (i.e. the tendency of the split pair once reunited not to come apart) has limited their use in the tracking of time dependent changes in analyte concentration. In this invention, the sensor molecule pulls them apart, provided an adequate bias has been selected for the hinge to open.

In another embodiment, the split molecule may comprise a biological active molecule that can be split into two or more parts, such that when the parts are brought back together in the presence of a target molecule of the sensor molecule, the function of the biological active molecule is restored.

The biological active molecule may comprise an active drug, a pro-drug, an enzyme, or a co-factor.

An embodiment of the invention providing a split molecule that is a biological active molecule is that a target effect can be provided. For example, an active drug can be provided upon the detection of a target molecule, such that the split molecule is brought back together and the active drug is provided. Localised or timed effects can be provided in this embodiment. For example, the split molecule may only become active when a target molecule is present. Therefore, the effect of the target molecule are latent until levels of the target molecule increase. For localisation, the split molecule may only be switched on in locations where a target molecule is present, for example only in specific cells, or cell compartments, or only in specific tissues that a target molecule is present.

In one embodiment, the activated drug may comprise an activated chemo-toxin.

In another embodiment, the split molecule may comprise a catalyst that initiates or enhances a chemical reaction in the presence of the target molecule. In another embodiment, the split molecule may comprise a molecule that releases heat through quenching in the presence of the target molecule and a suitable electro-magnetic field. In another embodiment, the split molecule may comprise a molecule that becomes an activated photosensitizer complex producing oxygen radicals in the presence of the target molecule and a light source.

In another embodiment, signal molecule B and signal molecule B′ may comprise reactive compounds that produce a chemical reaction in the presence of the target molecule.

The split molecule may comprise a toxin. For example the toxin may be the A and B components of an AB protein toxin, for example Diphtheria toxin. When A and B components of the toxin are brought together in the presence of a target molecule (for example on a target cell), the toxin is capable of binding and penetrating a target cell. In another example of the diphtheria toxin, the B component is split into two components B1 and B2 such that B1 remains fused to A. When A-B1 and B2 components of the toxin are brought together in the presence of the target molecule (for example on a target cell), the toxin A-B1-B2 is capable of binding and penetrating a target cell.

In some embodiments, the roles/function of molecules A and A′ relative to molecules B and B′ may be reversed. In the hinge, A may substantially oppose B or B′. Similarly A′ may substantially oppose B or B′. In one embodiment, molecule A or A′ will be positioned further from the joint molecule C relative to B or B′. In an alternative embodiment, molecule B or B′ will be positioned further from the joint molecule C than A or A′. The skilled person can design the relative positioning of the molecules, A, A′, B and B′ to suit the particular target molecule and sensor system required.

The spacer molecule may comprise a peptide, such as a polypeptide. In one embodiment, the spacer molecule may also contribute to the energy to bias the hinge apart. The spacer molecule may comprise a flexible polymer or a rigid polymer or rod. The length and flexibility of the spacer molecule may be designed/tuneable to accommodate the hinge open and closed dimensions for any given sensor molecule. The length of the spacer molecule may not exceed the sum of the lengths of each arm of the hinge, half of that value is the typical choice. For example, the ON state may be controlled by providing a higher flexibility to the spacer molecule in order for it to flex into an appropriate position sufficient for interaction of B and B′ to provide a signal. In one embodiment the spacer molecule comprises or consists of the sequence [GSG]^{m} or the sequence A[GSG]^{m}A (SEQ ID NO: 114), wherein m is 1, 2, 3, 4, 5, or more.

The rod-like molecules L and R may have a high aspect ratio (i.e substantially longer than it's width). For example, the aspect ratio of the rod-like molecules L and R may be about 6-10:1 (length to width). The aspect ratio of the rod-like molecules L and R may be at least about 6:1 (length to width). This is to ensure that the sensor will not significantly interfere with the chemistry it is designed to monitor (or in the case of drug/toxin delivery, be precise in its manipulation of the endogenous system). In one embodiment, the rod-like molecules L and R may each be at least 40 Ångströms in length. In another embodiment, the rod-like molecules L and R may each be at least 50 Ångströms in length. In another embodiment, the rod-like molecules L and R may each be at least 60 Ångströms in length. The rod-like molecules L and R may each be between about 40 and about 100 Ångströms in length. Alternatively, the rod-like molecules L and R may each be between about 50 and about 100 Ångströms in length. Alternatively, the rod-like molecules L and R may each be between about 60 and about 100 Ångströms in length. Alternatively, the rod-like molecules L and R may each be between about 60 and about 90 Ångströms in length. Alternatively, the rod-like molecules L and R may each be between about 60 and about 80 Ångströms in length.

The rod-like molecules L and R may each be between about 6 and about 8 Ångströms in width. The rod-like molecules L and R may each be between about 40 and about 100 Ångströms in length and between about 6 and 8 Ångströms in width. In another embodiment, the rod-like molecules L and R may each be between about 50 and about 100 Ångströms in length and between about 6 and 8 Ångströms in width. In another embodiment, the rod-like molecules L and R may each be between about 60 and about 100 Ångströms in length and between about 6 and 8 Ångströms in width. In another embodiment, the rod-like molecules L and R may each be between about 60 and about 90 Ångströms in length and between about 6 and 8 Ångströms in width. In another embodiment, the rod-like molecules L and R may each be between about 60 and about 80 Ångströms in length and between about 6 and 8 Ångströms in width.

Furthermore, the rod-like molecules L and R may not be so flexible that the rod easily folds. For example, the rod-like molecules L and R may be substantially rigid. This can be measured in terms of the average length of the molecule (along the most extended axis) and the fluctuations from the average length. For example, the length of the rod-like molecules L and R should not fluctuate by more than 20% to 30%. The length of the rod-like molecules L and R may not fluctuate by more than 25%. The length of the rod-like molecules L and R may not fluctuate by more than 30%.

In one embodiment, the rod-like molecule L and/or rod-like molecule R may comprise a polypeptide. The polypeptide may form an alpha-helical structure. Therefore, in one embodiment, the rod-like molecule L and/or rod-like molecule R comprise or consist of an alpha-helical polypeptide.

In another embodiment, the rod-like molecule L and/or rod-like molecule R may comprise a carbon nanotube, which has been sufficiently treated or produced to be substantially hydrophilic, and therefore soluble under physiological conditions. A person skilled in the art can select or functionalise carbon nanotubes to match the solvent.

Advantageously, the alpha-helical structure of the rod-like molecules L and R provides an effective rod, which is sufficiently rigid to provide a biased hinge mechanism, when joined by joint molecule C. For example the rods are not flexible to a degree that they can conform to any space or shape under normal physiological conditions. For example, the rod-like molecules L and R may be sufficiently rigid such that they allow the sensor to flex and return to their original shape/conformation. The rigidity of rods can be defined in various ways including: the ratio of the variance of the length of the rod to its average length, or alternatively the Young's modulus. This can be measured through a variety of experimental and theoretical/simulation methods, for example Atomic Force Microscopy, single molecular FRET microscopy, and through molecular simulation.

In one embodiment, the rod-like molecule L and/or rod-like molecule R may comprise an alpha-helical structure of a Leucine Zipper, such as the GCN4 leucine zipper. In one embodiment, the rod-like molecule L and/or rod-like molecule R may comprise an alpha-helical structure of a BAR protein. In one embodiment, the rod-like molecule L and/or rod-like molecule R may comprise collagen.

The rod-like molecule L and/or rod-like molecule R may be about 42 amino acids in length, for example when (EAAAK (SEQ ID NO: 106))⁶ (see Boersma et. al. Nature Methods, 2015; DOI: 10.1038/nmeth.3257). The rod-like molecule L and/or rod-like molecule R may be between about 35 and about 60 amino acids in length. The rod-like molecule L and/or rod-like molecule R may be between about 40 and about 60 amino acids in length. The rod-like molecule L and/or rod-like molecule R may be between about 40 and about 50 amino acids in length. The rod-like molecule L and/or rod-like molecule R may be between about 40 and about 45 amino acids in length.

The rod-like molecule L may comprise a number N of constituent molecules q1, q2, . . . , qN. The rod-like molecule R may comprise a number N′ of constituent molecules q′1, q′2, . . . , q′N′.

q1, q2, . . . , qN, q′ 1, q′2, . . . , q′N′ may be selected to be charged amino acids, or hydrophilic or hydrophobic amino acids, or a combination thereof. For example, the rod-like molecules L and R may comprise an alpha-helical polypeptide, which comprises qN, wherein q is charged, hydrophilic or hydrophobic amino acids, and N is the number of such amino acids typically between 1 and 20.

The rod-like molecules L and R may each comprise separate clusters of constituent molecules (q). For example, the rod-like molecules L and R may each comprise a cluster of constituent molecules q1 and a second cluster of constituent molecules q2. Two, three, four, five, six, seven, eight, nine, ten, eleven, twelve or more clusters (q) may be provided. Each constituent molecule cluster q may comprise the same number of residues and/or the same sequence of residues.

In one embodiment, the rod-like molecules L and R of the sensor molecule are symmetrical. For example, they may comprise similar or identical sequences. For example they may mirror each other when opposed in the hinge of the sensor molecule. Where the rod-like molecule L comprises a specified sequence, the rod-like molecule R may comprise the same sequence in reverse (i.e. a sequence running N to C terminal on one rod would be the same sequence on the opposing rod as the sequence running C to N terminal). The cluster of constituent molecules q of rod-like molecule L may align with the cluster of constituent molecules q of rod-like molecule R, such that they oppose each other in the hinge.

In an embodiment wherein the rod-like molecules L and R of the hinge comprise alpha-helices, the appropriate residue sequences may be provided by the skilled person using information in the literature regarding alpha helices and their stability, for instance, it is common general knowledge for the skilled person that the residue alanine has the highest tendency to form alpha helices when combined together.

Further examples of selection criteria that like charges at positions i, and i+4 in the peptide sequence should be avoided; and that the effective charges of the residues depend on their pKₐ values and the pH of the solvent. The skilled person will have access to publically available predictive tools online allowing the skilled person to assess the likely stability and solubility of the hinge at different pH conditions. Once a sequence for an appropriate alpha helix is selected, the corresponding structure can be built (as a pdb coordinate file) using bio-informatics tools available online. For example, the server known as IntFold may be used. All of the different peptides are combined together using protein alignment tools such as Modeller. The simulation codes used can be open source, free, and are commonly used in theoretical chemistry/biophysics to estimate free energy properties, with the most commonly used examples in biophysics being NAMD, and GROMACS. The typical fore-fields used in bio-simulations, including in this invention, is CHARMM27 and CHARMM36 including CMAP or Amber.

The choice of residues may be tailored by the skilled person to suit: (a) the solvent or fluid containing the sensor molecule (typically physiological conditions of temperature, pressure, pH and salt, but other conditions may pertain in for example in assays) and (b) the ligand binding and sensor domains binding energy in the ON state—values of which are known in the art (as binding affinities) (see for example table 1 herein) in embodiments wherein the target molecule (ligand) and target binding molecule A are each primary antibodies for specific antigens and their corresponding epitopes. Binding affinities (K_{D}) are typically of the order of μM, and correspond to a Gibbs free energy of the order of 8.5 kcal/mol. Binding affinities (K_{D}) may also be in mM to the nM range.

In one embodiment, the bias energy associated with the hinge opening is 5-20 kcal/mol. In another embodiment, the bias energy associated with the hinge opening is 8-15 kcal/mol. In another embodiment, the bias energy associated with the hinge opening is 8-12 kcal/mol. In another embodiment, the bias energy associated with the hinge opening is 10-12 kcal/mol.

The skilled person will understand that the binding energy of A and A′ may be substantially similar to the opposing bias energy of the hinge. Such comparable energies will prevent the hinge from being always closed or always open in the presence of the target molecule (e.g. it allows the dynamic switching between the two open and closed states of the hinge in the ON state).

The hinge may be biased into the open position by the constituent molecules. In particular, the constituent molecules of the rod-like molecule L may repel the constituent molecules of the rod-like molecule R in the presence of the solvent.

In one embodiment, the rod-like molecules L and R comprise an alpha helix of the following repeat residues [EAAAK(SEQ ID NO: 106)]^{m} and [EAAAK(SEQ ID NO: 106)]^{m} respectively, where m is the number of repeats ranging from 6 to 12, and E and K are positively charged at physiological pH condition (e.g. about pH 7.3). In one embodiment, the rod-like molecules L and R comprise an alpha helix of the following repeat residues [EAAAK(SEQ ID NO: 106)]^{m} and [KAAAE(SEQ ID NO: 107)]^{m} respectively, where m is the number of repeats ranging from 6 to 12, and E and K are positively charged at physiological pH condition (e.g. about pH7.3).

In one embodiment, the rod-like molecules L and R comprise an alpha helix of the following repeat residues [EAAAAK(SEQ ID NO: 108)]^{m} and [EAAAAK(SEQ ID NO: 108)]^{m} respectively, where m is the number of repeats ranging from 6 to 12, and E and K are positively charged at physiological pH condition (e.g. about pH7.3). In one embodiment, the rod-like molecules L and R comprise an alpha helix of the following repeat residues and [EAAAAK(SEQ ID NO: 108)]^{m} and [KAAAAE(SEQ ID NO: 109)]^{m} respectively, where m is the number of repeats ranging from 6 to 12, and E and K are positively charged at physiological pH condition (e.g. about pH7.3).

In one embodiment, the rod-like molecules L and R comprise an alpha helix of the following repeat residues [EAAAAAK(SEQ ID NO: 110)]^{m} and [EAAAAAK(SEQ ID NO: 110)]^{m} respectively, where m is the number of repeats ranging from 6 to 12, and E and K are positively charged at physiological pH condition (e.g. about pH7.3). In one embodiment, the rod-like molecules L and R comprise an alpha helix of the following repeat residues [EAAAAAK(SEQ ID NO: 110)]^{m} and [KAAAAAE(SEQ ID NO: 111)]^{m} respectively, where m is the number of repeats ranging from 6 to 12, and E and K are positively charged at physiological pH condition (e.g. about pH7.3).

The rod-like molecules L and R may comprise the sequence EAAKAAKA (SEQ ID NO: 112), or the mirror sequence AKAAKAAE (SEQ ID NO: 113) at the end immediately adjacent to the joint molecule C. For example, the joint molecule C may be flanked according to the following sequence [EAAAAAK(SEQ ID NO: 110)]⁴ EAAKAAKA(SEQ ID NO: 112)-[Joint Molecule C]-AKAAKAAE(SEQ ID NO: 113) [KAAAAAE (SEQ ID NO: 111)⁴

The hinge may comprise or consist of the hinge of the FRET crowding sensor molecule of Boersma et. al. (Nature Methods, 2015; DOI: 10.1038/nmeth.3257), or parts thereof.

The sensor molecule may be unimolecular (i.e. a unimolecular sensor). The sensor molecule may be a fusion protein.

In one embodiment the sensor molecule comprises the sequence of SEQ ID NO: 1. In another embodiment the sensor molecule comprises the sequence of SEQ ID NO: 2.

In one embodiment the sensor molecule comprises the rod-like molecules L and R of the sequence of SEQ ID NO: 1 or SEQ ID NO: 2. In another embodiment the sensor molecule comprises the joint molecule C of the sequence of SEQ ID NO: 1 or SEQ ID NO: 2. In another embodiment the sensor molecule comprises the rod-like molecules L and R, and the joint molecule C of the sequence of SEQ ID NO: 1 or SEQ ID NO: 2.

In one embodiment, the sensor molecule comprises or consists of the protein sequence FP1-A[GSG]^{m1} A (SEQ ID NO: 114)-TBM-A[GSG]^{m2} A L (SEQ ID NO: 114)-[hinge]-R A[GSG]^{m3} A (SEQ ID NO: 114)-BM-A[GSG]^{m4} A (SEQ ID NO: 114)-FP2,
wherein FP1 and FP2 are a signal molecule B and B′ respectively; TBM and BM are the target binding molecule A and binding molecule A′ respectively;
L and R denote the Left and Right alpha helices of the hinge;
A, S, and G denote the amino acids Alanine, Glycine and Serine; and
m1, m2, m3 and m4 are appropriately selected number of repeats to ensure that the sensor is functional according to the invention.

In one embodiment ml may be an integer of between 1 and 10. Additionally or alternatively, m2 may be an integer of between 1 and 10.

In one embodiment m =6-9 so that the biased energy of the hinge to be open, is equal to or lower than the binding energy of the target binding molecule A and binding molecule A′ in the presence of the target molecule. In several embodiments the hinge [EAAAK (SEQ ID NO: 106)⁶[SGS][KAAAE(SEQ ID NO: 107)]⁶ can be used which has a biased energy of 12 kcal/mol. This biased energy can be reduced by addition of flexible spacers. The typical reduction in the bias is in the range of 0.05 to 0.15 kcal/mol per Angstrom length of the spacer molecule. The skilled person will readily find the appropriate biased energy of the hinge by simulation or experimentation. Similarly, the binding energy of the target binding molecule A and binding molecule A′ in the presence of the target molecule will be known in the prior art (for example, if using a known ligand binding system) or estimated through experiment or molecular simulation by the skilled person.

In one embodiment, the rod-like molecules L and R and joint molecule C (the hinge) are composed of residue sequences such as:
[EAAAK (SEQ ID NO: 106)]ⁿ A[joint molecule C]^{m} A [KAAAE(SEQ ID NO: 107)]ⁿ; or
[EAAAK (SEQ ID NO: 106)]ⁿ A[joint molecule C]^{m} A [KAAAE (SEQ ID NO: 107)]ⁿ,
wherein E, A, G, S, and K are the single letter codes for amino acids and n and m are non-zero positive integers.

In one embodiment, n ranges from 12 to 24.

In one embodiment a rod-like molecule L or R may comprise the sequence [EAAAAAK(SEQ ID NO: 110)]⁴ EAAKAAKA(SEQ ID NO: 112). The rod-like molecule L and R together with the joint molecule C may comprise the sequence [EAAAAAK(SEQ ID NO: 110)]⁴ EAAKAAKA(SEQ ID NO: 112) S G S AKAAKAAE(SEQ ID NO: 113) [KAAAAAE(SEQ ID NO: 111)]⁴.

The rod molecules L and R may be neutral. Alternatively, rod molecules L and R may have a substantially low overall charge. In the example directly above the overall net charge is +2 electron Coulomb, under physiological conditions. The overall net charge may be no more than +1, +2, +3, +4, +5, or +10. The rod molecules L and R and/or the joint molecule C may be hydrophilic.

The joint molecule C may be flexible. The joint molecule C may comprise or consist of amino acids. The joint molecule C may comprise or consist of the amino acid glycine. The joint molecule C may comprise the amino acid sequence SGS or GS. In another embodiment, the joint molecule C may comprise a repeat of SGS or SG, for example [SGS]^{m} or [SG]^{m}, wherein m is selected from 2, 3, 4, 5, 6, 7, 8, 9, or 10.

The target binding molecule A may comprise an antibody, antibody fragment or mimic thereof. The target binding molecule A may comprise an antigen, for example a protein or peptide, which is capable of being bound by an antibody. The target binding molecule A may comprise a receptor protein, which comprises a ligand binding site. Alternatively, the target binding molecule A may comprise a ligand, which is capable of being bound by a receptor molecule.

The target binding molecule A may comprise nucleic acid.

The binding molecule A′ may comprise an antibody, antibody fragment or mimic thereof. The binding molecule A′ may comprise an antigen, for example a protein or peptide, which is capable of being bound by an antibody.

In one embodiment the target binding molecule A and binding molecule A′ are selected to each be primary antibodies targeting different epitopes on the same target, such as an analyte or antigen. In one embodiment, the target binding molecule A is a primary antibody targeting an analyte or antigen, and binding molecule A′ is the corresponding secondary antibody. In another embodiment, the target binding molecule A is an antigen targeting a primary antibody, and binding molecule A′ is the corresponding secondary antibody.

The target may comprise amino acid, such as a peptide or protein. In another embodiment, the target may comprise nucleic acid. The target may comprise a small molecule (i.e. having a MW of less than 900 KDa). The target may comprise a specific epitope. The target may comprise a complex of two or more molecules, such as a complex of sub-units.

The target may comprise a ligand, a receptor, an analyte, an antibody, or an enzyme, or fragments thereof. In one embodiment, the target binding molecule A and the binding molecule A′ may bind separate target molecules, wherein the separate target molecules are capable of associating with each other, for example to form a complex. The separate target molecules may be capable of associating with each other directly or via one or more intermediate molecules.

The target molecule may comprise a photon, such that the sensor molecule is capable of detecting light. For example Dronpa is capable of a conformational change in the presence of a photon. Therefore, target binding molecule A may comprise a light sensitive protein such as Dronpa, and upon conformational change in the presence of a photon, the binding molecule A′ may bind to the light sensitive molecule.

In one embodiment the target molecule may comprise glycated hemoglobin and/or glycated albumin. These targets in effect measure the long-time average concentration amount of glucose in an individual's blood, and are excellent indicators of the possible future onset of diabetes, as well as the indicators of how well a given therapy is progressing. Currently these are measured using immunoassays, but could be measured using the sensor molecule of the invention. The appropriate antibodies of each of these target molecules are well known in the art for providing binding molecules A and/or A′.

The sensor molecule according to the invention may be bound to another sensor molecule. For example, a first sensor molecule according to the invention may be bound to a second sensor molecule according to the invention (i.e. a pair of sensor molecules are provided together). The binding may be via any suitable polymer (e.g. polypeptide or nucleic acid), which is capable of associated the two sensor molecules together.

The first and second sensor molecules may be different. For example directed to a different target and/or producing a different signal or effect.

An advantage of providing two sensor molecule bound together is that they may be co-located for a particular assay, or to carry out a specific function in the same area. For example, one sensor molecule may be responsible for reporting/detecting the presence of a target molecule and the other sensor molecule may be responsible for carrying out a reaction.

The sensor molecule may comprise combinations of functions provided by signal molecules B and B′. For example a single sensor molecule may comprise the target binding molecule A and binding molecule A′, and two or more sets of sensor molecules B and B′. The two or more sets of B and B′ molecules may be different in function. For example, one set may provide a signal function, such as a fluorescence signal, and the second set may provide a split molecule, such as a biological active. Therefore, the working sensor may be visualised as it provides the additional function.

Two or more sensors can be joined in unison to give rise to a collective effect. For example the sensors may form a channel, which opens in the presence of the target molecule allowing passage of other small molecules and conversely in absence of the target molecule prevents there passage. In another embodiment two or more sensors in unison can be used as a scaffold to transport a drug like molecule to a cellular compartment, and in the presence of an analyte open and releases the drug.

According to another aspect of the present invention, there is provided a nucleic acid encoding the sensor molecule of the invention herein.

In one embodiment, the entire sensor molecule may be encoded as a fusion protein. In another embodiment, parts of the sensor molecule may be encoded, for example such that the remaining components can be added at a later stage to form the complete sensor molecule.

The nucleic acid may comprise or consist of a vector. The vector may be an expression vector arranged to express the sensor molecule in a host cell.

According to another aspect of the present invention, there is provided a host cell comprising the nucleic acid according to the invention herein and/or the sensor molecule according to the invention herein.

The host cell may be capable of expressing the sensor molecule of the invention herein.

According to another aspect of the present invention, there is provided an assay method for the detection of a target molecule in sample comprising:
providing the sample;
providing the sensor molecule according to the invention in the sample;
detecting the presence or absence of a signal from the sensor molecule;
   wherein an ON signal confirms the presence of the target molecule in the sample.

The assay method may further comprise determining the level/intensity of the signal. The assay method may further comprise determining the presence or level/intensity of the signal over time. The assay method may further comprise determining the location of the signal.

The sample may comprise biological fluid sample, such as blood, serum, blood plasma, urine, faeces, aspirate, biopsy, growth media, or an environmental sample, such as a water sample.

According to another aspect of the present invention, there is provided a composition comprising the sensor molecule according to the invention.

The composition may comprise two or more different sensor molecules according to the invention. The different sensor molecule may differ in the target molecule being detected, and/or the light signal produced. For example different fluorescent or bioluminescent molecules may be provided on different sensor molecules, which provide different colour light signals.

The composition may be a pharmaceutical composition. The pharmaceutical composition may comprise any pharmaceutically acceptable excipients.

According to another aspect of the present invention, there is provided an assay method for the detection of a target molecule in vivo comprising:
providing the sensor molecule according to the invention in vivo;
detecting the presence or absence of a signal from the sensor molecule; wherein an ON signal confirms the presence of the target molecule in vivo.

In vivo may comprise in a cell, such as a prokaryote cell or eukaryote cell. In vivo may comprise extracellular environment, for example in a tissue or fluid.

The sensor molecule may be provided in vivo by expression of the sensor molecule in vivo, for example in a cell.

The sensor molecule may be attached and or embedded to a protein, protein matrix, capsid, a cell membrane, sub-cellular membrane, or an organic/inorganic substrate.

According to another aspect of the present invention, there is provided the use of the sensor molecule according to the invention to visualise or monitor any of the following: (a) the structure and conformation of proteins; (b) the spatial distribution and assembly of protein complexes; (c) protein receptor/ligand interactions including the local concentrations of analytes; (d) the interactions of single molecules; (e) the structure and conformations of nucleic acids; (f) the distributions and transport of lipids; (g) membrane potential sensing; (h) monitoring fluorogenic protease substrates; (i) local cellular concentrations of cyclic AMP and calcium.

According to another aspect of the present invention, there is provided the use of the sensor molecule according to the invention in the detection of a target analyte, and optionally its concentration, in assays or living cells.

The use may involve tracking the target analytes over time.

According to another aspect of the present invention, there is provided the use of the sensor molecule according to the invention as a drug or drug delivery vehicle to, or within, biological cells, fluids or tissue.

The drug may be a chemo-toxin.

According to another aspect of the present invention, there is provided the use of the sensor molecule according to the invention to provide or catalyse a chemical reaction in the vicinity or within biological cells, organic materials, fluids or tissue.

According to another aspect of the present invention, there is provided the use of the sensor molecule according to the invention to deliver heat in the vicinity or within biological cells, fluids, tissue or organic materials.

According to another aspect of the present invention, there is provided the use of the sensor molecule according to the invention in photodynamic therapy in the vicinity or within biological cells, fluids, tissue, or organic materials.

According to another aspect of the present invention, there is provided the use of the sensor molecule according to the invention for cell killing, wherein the sensor molecule comprises a split molecule that is an active toxin once the parts of the split molecule are brought together in the presence of a target molecule, optionally wherein the target molecule is specific to the cell or cell type.

The uses of the invention may be in vivo or in vitro.

According to another aspect of the present invention, there is provided the use of the sensor molecule according to the invention to perform assays for analytes including titration measure using microtiters or vials, with and without specialised equipment. Such use may have multiple applications including environmental, health, food safety, and security.

According to another aspect of the present invention, there is provided the use of the sensor molecule according to the invention to detect analytes in suitable continuous flow chambers. Such use may have multiple applications including environmental, health, food safety, and security.

According to another aspect of the present invention, there is provided a method of providing a biological active only in the presence of a target molecule comprising:
providing the sensor molecule according to the invention, wherein the sensor molecule comprises a split molecule, wherein the split molecule is a biological active.

According to another aspect of the present invention, there is provided a method of treatment for a disease in a subject comprising the administration of the sensor molecule or composition according to the invention to a subject, wherein the sensor molecule comprises a biological active in the form of a split molecule, which is capable of becoming an active suitable for treatment of the disease.

The disease may comprise cancer. In an embodiment wherein the disease is cancer, the biological active may comprise a chemo-toxin. The biological active may comprise a AB-toxin. In an embodiment wherein the disease is cancer, the target molecule may comprise a cancer cell specific receptor or cell surface marker.

According to another aspect of the present invention, there is provided a method of treatment for a disease in a subject comprising the administration of the sensor molecule or composition according to the invention to a subject, wherein the sensor molecule comprises a split molecule, which is capable of becoming an active suitable for treatment of the disease in the presence of a target molecule.

The subject may be mammalian. The subject may be human.

In embodiments related to sequences, the skilled person will understand that there can be some sequence variation without substantially affecting or removing the intended function of the sequence. Such variations include mutations, additions, deletions and substitutions of residues or nucleotides. Conservative substitutions may be made. In some embodiment, the sequence can have at least 80% identity with the listed sequence. In another embodiment, the sequence may have 85%, 90%, 95%, 98% or 99% sequence identity. Such variants are within the scope of the invention. Sequence identity may be determined using standard NCBI BLASTp or BLASTn parameters.

### Definitions

The terms “close proximity” or “near” is understood to mean physical interaction, such as binding, or sufficiently close for the intended function of the molecule. For example, sufficiently close for fluorescence excitation to occur between FRET molecules (for example about 40-60 Ångströms or less). In applications requiring closer proximity, such as in the use of a split molecule (such as a chemo-toxin), the distance may be considered to be close enough for the split molecule to function (e.g. less than 40 Ångströms or less than 20 Ångströms). The skilled person will understand that the distance can vary between different sensor molecule functions and components.

The term “open position” in regard to the hinge is understood to mean that the hinge is apart such that any binding or signal function between molecules, such as FRET, does not occur. The open position may require the molecules A and A′ and/or B and B′ to be at least 60 Ångströms apart. The term “closed position” in regard to the hinge is understood to mean that the hinge is together such that any binding or signal function, such as FRET, can occur. The closed position may require the molecules A and A′ and/or B and B′ to be no more than 30 Ångströms apart, or no more than 20 Ångströms apart, alternatively, no more than 10 Ångströms apart.

The term “attracted” used herein is understood to mean that one molecule is drawn towards another molecule through either direct binding or indirect binding, or brought into direct contact or closer proximity.

The sensor molecule may be capable of detection of the target molecule under physiological conditions and/or assay conditions. Furthermore, reference to binding, affinity, attraction, biasing energies or similar molecular interactions may be under physiological conditions and/or assay conditions. The term “physiological conditions” is understood to include physiological pH, physiological salt concentrations, and physiological temperature. The sensor molecule may also be required to work in in vitro assay conditions. Such assay conditions may match physiological conditions, for example substantially similar to intracellular or extracellular conditions in vivo. The skilled person can readily adjust the sensor molecule constituents and features within the scope of the invention in order to provide function in any given assay conditions. For example such changes may be based on known molecule pK values.

The sensor molecule may also be referred to as a “Tuneable Multistate Dynamical Unimolecular Hinge Sensor”.

Within this document, the terms “target molecule”, “ligand” and “analyte” are equivalent and will be used interchangeably, reflecting their usage in the literature.

The terms “connected”, “attached” or “attachment” may include a covalent binding.

The term “antibody” as used herein refers to immunoglobulin molecules and immunologically active portions of immunoglobulin molecules, i.e., molecules that contain an antigen binding site that specifically binds an antigen, whether natural or partly or wholly synthetically produced. The term also covers any polypeptide or protein having a binding domain which is, or is homologous to, an antibody binding domain. These can be derived from natural sources, or they may be partly or wholly synthetically produced. Examples of antibodies are the immunoglobulin isotypes (e.g., IgG, IgE, IgM, IgD and IgA) and their isotypic subclasses; fragments which comprise an antigen binding domain such as Fab, scFv, Fv, dAb, Fd; and diabodies. Antibodies may be polyclonal or monoclonal. A monoclonal antibody may be referred to as a “mAb”.

It is possible to take monoclonal and other antibodies and use techniques of recombinant DNA technology to produce other antibodies or chimeric molecules which retain the specificity of the original antibody. Such techniques may involve introducing DNA encoding the immunoglobulin variable region, or the CDRs, of an antibody to the constant regions, or constant regions plus framework regions, of a different immunoglobulin. See, for instance, EP-A-184187, GB 2188638A or EP-A-239400. A hybridoma or other cell producing an antibody may be subject to genetic mutation or other changes, which may or may not alter the binding specificity of antibodies produced.

As antibodies can be modified in a number of ways, the term “antibody” should be construed as covering any specific binding member or substance having a binding domain with the required specificity. Thus, this term covers antibody fragments, derivatives, functional equivalents and homologues of antibodies, humanised antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023. A humanised antibody may be a modified antibody having the variable regions of a non-human, e.g., murine, antibody and the constant region of a human antibody. Methods for making humanised antibodies are described in, for example, U.S. Pat. No. 5,225,539.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment [25] which consists of a VH domain; (v) isolated CDR regions; (vi) F(ab′)2 fragments, a bivalent fragment comprising two linked Fab fragments; (vii) single chain Fv molecules (scFv), wherein a VH domain and a VL domain are linked by a peptide linker which allows the two domains to associate to form an antigen binding site; (viii) bispecific single chain Fv dimers (PCT/US92/09965) and; (ix) “diabodies”, multivalent or multispecific fragments constructed by gene fusion (WO94/13804; [28]).

Diabodies are multimers of polypeptides, each polypeptide comprising a first domain comprising a binding region of an immunoglobulin light chain and a second domain comprising a binding region of an immunoglobulin heavy chain, the two domains being linked (e.g., by a peptide linker) but unable to associated with each other to form an antigen binding site: antigen binding sites are formed by the association of the first domain of one polypeptide within the multimer with the second domain of another polypeptide within the multimer (WO94/13804).

Where bispecific antibodies are to be used, these may be conventional bispecific antibodies, which can be manufactured in a variety of ways, e.g., prepared chemically or from hybrid hybridomas, or may be any of the bispecific antibody fragments mentioned above. It may be preferable to use scFv dimers or diabodies rather than whole antibodies. Diabodies and scFv can be constructed without an Fc region, using only variable domains, potentially reducing the effects of anti-idiotypic reaction.

Bispecific diabodies, as opposed to bispecific whole antibodies, may also be useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected.

Any known antibody or fragment thereof that is capable of specifically binding to the desired target molecule may be used in the sensor molecule according to the invention.

Reference herein regarding the binding of a molecule to a target molecule may be considered as specific binding. “Specific” is generally used to refer to the situation in which one member of a specific binding pair will not show any significant binding to molecules other than its specific binding partner(s), and, e.g., has less than about 30% cross reactivity with any other molecule. In other embodiments it has less than 20%, 10%, or 1% cross reactivity with any other molecule. The term is also applicable where e.g., an antigen binding domain is specific for a particular epitope which is carried by a number of antigens, in which case, the specific binding member carrying the antigen binding domain will be able to bind to the various antigens carrying the epitope.

In one embodiment, the signalling molecule may comprise the calcium-binding messenger protein Calmodulin. In one embodiment the novel biased hinge substitute's linkers in currently available unimolecular FRET sensors, and such that it is tuned to suitably match its FP's, ligand binding domain and sensor domain.

In another embodiment the novel biased hinge substitutes linkers in currently available unimolecular BRET sensors, and such that it is tuned to suitably match its FP's, ligand binding domain and sensor domain.

In another embodiment, the biased rigid hinge can be tuned for completely new FP's or ligand binding domain or sensor domains, but still in the context of FRET or BRET probes.

In another embodiment the curly element C depicted in FIGS. 2 and 3 is a highly flexible connected linker.

In another embodiment the curly element C depicted in FIGS. 2 and 3 is a combination of consecutive sequences of rigid and flexible peptides.

In another embodiment either the A or A′ end of the sensor (see FIGS. 2 and 3) is typically anchored via a short spacer to the C-terminus of a protein close to the cellular locale of interest.

In another embodiment A or A′ end of the sensor and fluorescent tags can be attached to target proteins using chemical labelling using covalent bonding (such as amine labelling, thiol labelling) (see reference NP24), enzymatic labelling (Labelling catalysed by post-translational enzyme modification, Labelling with self-modified enzymes like Cutinase or Interin) (see reference NP25) and non-covalent tagging (tetracysteine/biarsenical tag, histidine tag etc., see reference NP26).

In another embodiment FPs can have a variety of manifestations such as photoactivatable (PA-mRFP1, PA-mCherryl), photo-convertible (Kaede, Dendra2), photo-switchable (Dronpa, Pardon), photo-convertible/ photo-switchable (IrisFP) and Fluorescent Protein Timers (DsRed-E5, Fast-FT).

The use of multiple acceptor probes provides another embodiment of the sensor, where for instance, one or more acceptor FP's of different colours (but such that RET is possible) are placed on target proteins close to the sensor donor FP. This allows relative changes in position of the FP's to be measured at essentially the same time as variations in the local analyte concentration.

In one embodiment the invention can be used to visualise the structure and conformation of proteins.

In another embodiment the invention can be used to monitor the spatial distribution and assembly of protein complexes.

In another embodiment the invention can be used to monitor receptor/ligand interactions in proteins.

In another embodiment the invention can be used for probing interactions of single molecules.

In another embodiment the invention can be used for probing structure and conformation of nucleic acids.

In another embodiment the invention can be used for monitoring distribution and transport of lipids.

In another embodiment the invention can be used for membrane potential sensing.

In another embodiment the invention can be used for monitoring fluorogenic protease substrates.

In another embodiment the invention can be used for monitoring cyclic AMP, cyclic GMP, calcium, zinc, and Halide ions.

In another embodiment the invention can be used as redox sensors, pH sensors.

In another embodiment the invention can be used as phosphatase activity sensor, and histone acetylation/methylation sensors.

In another embodiment the invention can be used to measure the concentration of glycated haemoglobin in blood.

In another embodiment the invention can be used to measure the concentration of glycated albumin in blood.

In another embodiment the invention can be used to measure the concentration of blood clotting factors 1 to 11.

In another embodiment the invention can be used to measure growth factor such as Epidermal growth factor, fibroblast factor, vascular endothelial growth factor.

In another embodiment the invention can be used to measure insulin, insulin-like growth factor and oxytocin, and steroid hormones.

In another embodiment the invention can be used as cell cycle reporter.

In another embodiment the invention can be used as strain sensors.

In another embodiment the invention can be used as sugar sensors.

In another embodiment the invention can be used in high-throughput screening drug discovery.

In another embodiment the invention can be used in high-throughput screening of agonist and antagonist ligands of taste and olfactory receptors.

In another embodiment the invention can be used in the observation of the turnover of selected metabolites at the single cell level in real time.

In another embodiment the invention can be used in the visualization of specific macromolecular machines within the cellular environment.

In another embodiment the invention can be used to determine the effectiveness of agonist or antagonist ligands acting on G protein receptors.

In another embodiment the invention can be used as light/ligand activated sensors or targeted drug discovery.

In another embodiment the invention can be used as actuators or active agents in the manipulation and control of biological processes and signalling networks.

In another embodiment the invention can be used as an organic or inorganic indicator of the presence and concentration of analytes in analytical chemistry, biochemistry, photochemistry, food, health, and environmental sciences.

In an embodiment where the invention is composed of inorganic or organic components or mixtures thereof, it can be used as electronic sensors, nano-electromechanical systems, memory devices and nano-actuators.

In another embodiment the invention is be used to estimate the binding energies of different ligands to receptors and rank their efficacy as agonist and antagonists with applications in the development of drugs, flavours, perfumes, insectacides, with applications for human, animal and plant health, and the food and perfume indistries.

In another embodiment of the invention the detection of analytes such as antigens is made through macromolecules A and A′. Macromolecules A and A′ can be selected to be primary antibodies targeting different epitopes on the analyte, which may the same type of epitope but at different locations. Macromolecules B and B′ can each consist of one or more selected molecules or moieties of split molecules, which when brought close together produce a variety of selected effects: (a) resonance energy transfer in the presence of a suitable electro-magnetic field; (b) fluorescence in the presence of a suitable electro-magnetic field; (c) bioluminescence; (d) activated drug; (e) activated chemo toxin; (f) chemical reaction; (g) catalysed chemical reaction; (h) in the presence of a suitable electro-magnetic field the release of heat through quenching; (i) in the presence of a suitable electro-magnetic field either of an external or endogenous source, the production of reactive oxygen. In addition several of these effects can be combined in the same sensor. These effects take place in the vicinity on the sensor, which can be close to or within cells, cellular compartments, or in vitro.

Another embodiment of the invention can be used in the detection and measurement of the concentration of analytes in assays and in living cells and their tracking over time using pairs of primary antibodies or fragments thereof targeting epitopes on corresponding antigens.

Another embodiment the invention can be used in the detection of analytes and measurement of the concentration in immunoassays and in living cells and their tracking over time, using a primary antibody or suitable fragments thereof targeting an epitope, and a corresponding secondary antibody.

Another embodiment of the invention can be used in the detection and measurement of concentration of primary antibodies in immunoassays and in living cells and their tracking over time using a corresponding antigen or antigen fragment and a corresponding secondary antibody.

Another embodiment of the invention can be used in the activation of pharmaceutically active molecules and toxins on the detection of target analytes on and within living cells.

Another embodiment of the invention can be used in the activation of pharmaceutically active molecules and chemo-toxins, catalysts and other chemical reactions on the detection of target analytes on and within living cells, and simultaneous measurement including optical marking of the location and of said analytes, and their tracking over time.

Another embodiment of the invention can be used on the presence of target analytes for the heating of local cellular and subcellular regions.

Another embodiment of the invention can be used for photodynamic therapy targeting, for example, cancerous cells and various pathogens in living tissue.

Another embodiment of the invention can be used in the field for immunoassays for analytes using microtiters or vials, and without specialised equipment, and at low cost for several applications including environmental, health, food safety, and security.

Another embodiment of the invention can be used in the field immunoassays for analytes including titration measurements using microtiters or vials, with and without specialised equipment, and at low cost for several applications including environmental, health, food safety, and security.

Another embodiment of the invention can be used in the detection of analytes in suitable continuous flow chambers for several applications including environmental, health, food safety, and security.

### Applications

The sensor molecule according to the invention may be used in any one or more of the following applications.

Kits for bio-chemistry/bio-molecular/molecular medicine research.

Pharmaceuticals and Biopharmaceuticals—Drug discovery—including high throughput discovery for human and animal health. FRET assays are often used with additional techniques like robotic, ultra-high throughput screening systems to screen for potential drugs. The sensitivity of these assays can be increased using the sensor or the invention, thereby giving lower false positives.

Medical diagnostics—for human and animal health. FRET is used in designing diagnostic assays to measure analytes relevant to human health like insulin and growth factors etc. Our sensor can render them more accurate.

Food industry—including high throughput discovery of flavours and functional foods for humans and animals

Perfume and cosmetics industry—including high throughput discovery of perfumes

Biotechnology industry—Membrane fusion assays. Stuck et. al. showed the interaction of the lipid rafts could be studied as a function of addition of an analyte concentration, when a sensor domain embedded partially within a lipid rafts is attached to a FP and connected by a dynamical hinge to a FP and ligand binding domain either inside or outside the cellular compartment. Struck, D. K., D. Hoekstra, and R. E. Pagano. 1981. Use of resonance energy transfer to monitor membrane fusion. Biochemistry. 20: 4093-4099.

Biopharmaceuticals/pharmaceuticals industries—immunoassays. Immunoassay using flexible linkers are often used in assays. One can increase the signal to noise by using a dynamical hinge instead of a flexible linker.

Biopharmaceuticals/pharmaceuticals industries/medical diagnostics/forensics—automated DNA sequencing, and Real-time PCR assays and SNP detection, and detection of nucleic acid hybridization. PCR is often used as a tool to amplify the peptide based analyte. However the ability of the sensor of the invention to detect lower concentration of analytes will allow the diagnostic process to skip the intensive PCR step altogether.

Electronics, semiconductors industries, quantum computing

Health Sciences Industries—Development of testing kits in immunology for use in the health-science industry. In the case of enzyme-linked immunosorbent assay (ELISA) is often used to measure antibodies, antigens, proteins and glycoproteins, for example to diagnose HIV, test pregnancy, and measurement of cytokines or soluble receptors in cell supernatant or serum. ELISA assays are generally carried out in 96 well plates, allowing multiple samples to be measured in a single experiment. The sensor of the invention can make ELISA more accurate. Other assays include cell based (Lymphoproliferative assays to phytohemagglutinin (PHA), pokeweed mitogen (PWM) and Candida, Natural Killer Cell) and flow based assays (AssaysFlow cytometric, single platform CD4 counts).

Security industry, for example, detection of chemicals and explosives for security purposes). Explosive sensing often involves using FRET molecules where the explosive binds to the system and disrupts FRET. The sensor of the invention can be incorporated in such sensing to increase sensitivity. For example, PETN and RDX using a FRET-based fluorescence sensor system.

Food and Cosmetics industries—detection of pathogens and allergens in foods and cosmetics. FRET assays are often used for sensing toxins, or allergens in food. The sensor can be incorporated in such to increase sensitivity.

Assays in the environmental industry (e.g. testing of air and water purity, detection of pathogens and allergens in foods and cosmetics). FRET is used in detection of chemicals, which in even in minute quantities act as allergens etc. The sensor can be incorporated in such to increase sensitivity.

Life science associated industries: real time visualization of analytes in assays and in vivo. FRET is widely used to measure spatio-temporally the effect of signalling molecule like Calcium etc inside a live cell. The sensor can be incorporated in such to increase sensitivity.

Pharmaceutical industry—delivery and activation of drugs, catalysts and chemo-toxins, photodynamic therapeutics. Drug delivery is a priority area in biomedical sciences, our sensors both individually or in unison can be used to deliver drugs in presence of a target molecule.

The skilled person will understand that optional features of one embodiment or aspect of the invention may be applicable, where appropriate, to other embodiments or aspects of the invention.

Embodiments of the invention will now be described in more detail, by way of example only, with reference to the accompanying drawings.

### BRIEF DESCRIPTION OF DRAWINGS

FIG. 1 Schematic description of FRET unimolecular sensor using a flexible linker as described in reference PT1 by Matsuda et al. The OFF state of the sensor corresponding to the absence of the ligand or analyte is displayed in the left panel, where ligand binding domain and sensor domain are on average far apart, and as a consequence the RET signal intensity is low. The donor and acceptor fluorophore proteins are depicted as cylinders. The ON state of the sensor corresponding to the presence of the ligand or analyte is displayed in the right panel, where ligand binding domain and sensor domain are on average in close contact, and as a consequence the RET signal intensity is high.

FIG. 2 Schematic description of a multistate dynamical unimolecular hinge sensor of the present invention in the OFF state corresponding to the absence of the ligand.

FIG. 3 Schematic description of a multistate dynamical unimolecular hinge sensor of the present invention in the ON state sensor corresponding to the presence of the ligand or analyte.

FIG. 4 Test results for prototype 1 of a multistate dynamical unimolecular hinge sensor. The resonance energy transfer signal intensity I at physiological temperature is plotted as a function of binding energy Δε for different values of the depth of the bias Dₑ: =0; ▪=1Kcal/mol; ♦=3 Kcal;/mol; ▴=5 Kcal/mol.

FIG. 5 Test results for prototype 1 of a multistate dynamical unimolecular hinge sensor. The resonance energy transfer signal to noise ratio (I−I₀)/I₀ at physiological temperature is plotted as a function of binding energy Δε for different values of the depth of the bias Dₑ: =0; ▪=1Kcal/mol; ♦=3 Kcal/mol; ▴=5 Kcal/mol.

FIG. 6 Test results for prototype 1 of a multistate dynamical unimolecular hinge sensor, where the bias depth Dₑ=4 Kcal/mol. The probability distribution P(r) of inter macromolecule distances r at physiological temperature is plotted for different values of the binding energies Δε =1 Kcal/mol; ▪=3 Kcal/mol; ▴=5 Kcal/mol.

FIG. 7 Test results for prototype 1 of a multistate dynamical unimolecular hinge sensor, where the bias depth Dₑ=4 Kcal/mol. The probability P⁺(r) of inter macromolecule distances r at physiological temperature is plotted for different values of the binding energies Δε =1 Kcal/mol; ▪=3 Kcal/mol; ♦=5 Kcal/mol. Note that P⁺(r) is simply the integral from 0 to r of P(r) and is otherwise known as the cumulative probability. Here it gives the probability that two spheres of the model are within a distance r of each other.

FIG. 8 Test results for prototype 2 of a multistate dynamical unimolecular hinge sensor. The resonance energy transfer signal intensity I at physiological temperature is plotted as a function of binding energy Δε for different values of the depth of the bias Dₑ: =0; ▪=1 Kcal/mol; ♦=3 Kcal;/mol; ▴=5 Kcal/mol.

FIG. 9 Test results for prototype 2 of a multistate dynamical unimolecular hinge sensor. The resonance energy transfer signal to noise ratio (I−I₀)/I₀ at physiological temperature is plotted as a function of binding energy Δε for different values of the depth of the bias Dₑ:=0; ▪=1 Kcal/mol; ♦=3 Kcal/mol; ▴=5 Kcal/mol.

FIG. 10 Test results for prototype 2 of a multistate dynamical unimolecular hinge sensor, where the bias depth Dₑ=4 Kcal/mol. The probability distribution P(r) of inter macromolecule distances r at physiological temperature is plotted for different values of the binding energies Δε =1 Kcal/mol; ▪=3 Kcal/mol; ♦=5 Kcal/mol.

FIG. 11 Test results for prototype 2 of a multistate dynamical unimolecular hinge sensor, where the bias depth Dₑ=4 Kcal/mol. The probability P⁺ (r) of inter macromolecule distances r at physiological temperature is plotted for different values of the binding energies Δε =1 Kcal/mol; ▪=3 Kcal/mol; ♦=5 Kcal/mol.

FIG. 12 Test results for prototype 3 of a multistate dynamical unimolecular hinge sensor. The resonance energy transfer signal intensity I at physiological temperature is plotted as a function of binding energy Δε for different values of the depth of the bias Dₑ: =0; ▪=1 Kcal/mol; ♦=3 Kcal;/mol; ♦=5 Kcal/mol.

FIG. 13 Test results for prototype 3 of a multistate dynamical unimolecular hinge sensor. The resonance energy transfer signal to noise ratio (I−I₀)/I₀ at physiological temperature is plotted as a function of binding energy Δε for different values of the depth of the bias Dₑ: =0; ▪=1 Kcal/mol; ♦=3 Kcal/mol; ▴=5 Kcal/mol.

FIG. 14 Test results for prototype 3 of a multistate dynamical unimolecular hinge sensor, where the bias depth Dₑ=4 Kcal/mol. The probability distribution P(r) of inter macromolecule distances r at physiological temperature is plotted for different values of the binding energies Δε =1 Kcal/mol; ▪=3 Kcal/mol; ▴=5Kcal/mol.

FIG. 15 Test results for prototype 3 of a multistate dynamical unimolecular hinge sensor, where the bias depth Dₑ=4 Kcal/mol. The probability P⁺(r) of inter macromolecule distances r at physiological temperature is plotted for different values of the binding energies Δε =1 Kcal/mol; ▪=3 Kcal/mol; ♦=5 Kcal/mol.

FIG. 16 Schematic description of a primary antibody showing the heavy and light chains, the Fab and Fc regions, as well as the location of disulphide bonds and carbohydrates.

FIG. 17 Schematic description showing different fragments of a primary antibody realizable in experiments.

FIG. 18 Schematic description of Tuneable Multistate Dynamical Unimolecular Hinge Sensors, when A and A′ are primary antibodies, where the analyte is an antigen, and the system is free floating in the solution, and B and B′ are fluorophores or the corresponding moieties of a single split fluorophore or split bioluminescent molecule. The antigen is represented as a cone purely for illustrative reasons, but need not be of such a shape.

FIG. 19 Schematic description of Tuneable Multistate Dynamical Unimolecular Hinge Sensors, when A and A′ are primary and corresponding secondary antibodies, where the antigen acts like an analyte, and the system is free floating in the solution, and B and B′ are fluorophores or the corresponding moieties of a single split fluorophore or split bioluminescent molecule. The antigen is represented as a cone purely for illustrative reasons, but need not be of such a shape.

FIG. 20 Schematic description of Tuneable Multistate Dynamical Unimolecular Hinge Sensors, when A and A′ are the antigen and a secondary antibodies, where the primary antibody acts like an analyte, and the system is free floating in the solution, and B and B′ are fluorophores or the corresponding moieties of a single split fluorophore or split bioluminescent molecule. The antigen is represented as a cone purely for illustrative reasons, but need not be of such a shape.

FIG. 21 Schematic description of Tuneable Multistate Dynamical Unimolecular Hinge Sensors similar to FIG. 18 except that the sensor is not feely floating but instead is attached via one of the primary antibodies to a suitable surface.

FIG. 22 Schematic description of Tuneable Multistate Dynamical Unimolecular Hinge Sensors similar to FIG. 19 except that the sensor is not feely floating but instead is attached via the primary antibody or the secondary antibody to a suitable surface.

FIG. 23 Schematic description of Tuneable Multistate Dynamical Unimolecular Hinge Sensors similar to FIG. 20 except that the sensor is not feely floating but instead is attached via the antigen or the secondary antibody to a suitable surface.

FIG. 24 A publically available alignment code known as modeller, the visualiser code known as vmd, and the molecules simulation code known as GROMACS to relax the system in a box with water using periodic conditions to emulate bulk conditions under one atmosphere and physiological temperature and pressure. Experimental methods to build these sensors, once the protein sequence of the hinge and interconnecting flexible linkers are defined are in the literature and outlined in the prior art descriptions.

Snap shots of the two examples after a few nanoseconds simulation in water under normal temperature and pressure are attached (the water is not shown in order to visualise the sensor protein system). The first system comprises the fhal and pka substrate sensor and ligand binding domains combined with two fluorescent proteins interconnected by the biased hinge. The second system comprises the same fhal and pka substrate sensor and ligand binding domains combined with the two moieties of a split yellow fluorescent protein interconnected by the biased hinge, the latter having a smaller and more effective joint connecting the alpha helix proteins.

A few non-standard amino acids from the protein sequences were removed to facilitate full scale simulation using the CHARMM 27 and 36 force fields directly, but this does not affect the structure of the system.

First system (fhal-fpl-hinge-fp2-pka substrate, note that the “-” represent short flexible linkers A[GSG]ⁿ A (SEQ ID NO: 114) and these can be modified to optimize the functioning of the system). The protein sequence is SEQ ID NO: 1 herein.

Second system is hal-splitYFP1 -hinge-splitYFP2-pka substrate, note that the “-” represent short flexible linkers A[GSG]³A (SEQ ID NO: 115) and these can be modified to optimize the functioning of the system). Here splitYFP1 and splitYFP2 denote the two moieties of yellow fluorescent protein. The protein sequence is SEQ ID NO: 2 herein.

FIG. 25—Using the simulation methods described in the prior art and disclosed in P34, one can predict the bias energy associated with the hinge to be open to be 10-12 kcal/mol for the protein sequence above, and similar structures. For instance, [EAAAK (SEQ ID NO: 106)]⁶A[GS]²A [KAAAE(SEQ ID NO: 107)]⁶ illustrated in FIG. 25A which has the free energy as a function of distance between the end of the hinge displayed in FIG. 25B.

FIG. 26 Free energy as a function of distance (Angstrom) between the two end of the hinge [EAAAK(SEQ ID NO: 106)]⁶ SGS [KAAAE(SEQ ID NO: 107)]⁶.

FIG. 27 (FRET) Forkhead-associated fhal and protein kinase A substrate (PKA substrate with a pair of fluorescent proteins and the biased hinge).

FIG. 28 Sensor combining split YFP moieties with forkhead fhal and protein kinase A substrate and the hinge hinge [EAAAK(SEQ ID NO: 106)]⁶ SGS [KAAAE(SEQ ID NO: 107)]⁶.

FIG. 29 Sensor combining a pair of single domain camedloid antibodies with the moieties of a fluorescent protein and the biased hinge.

FIG. 30 Split AB toxin with a pair of single domain camedloid antibodies and the biased hinge. The AB toxin is split into two moieties: AB₁ and B₂. The particular division of B is made because B₂ is believed to be responsible for the binding of the toxin to the cell membrane preceding penetration of the toxin into the cell.

FIG. 31 Provides Examples 1 to 5 of sequences of the full sensor system. These examples serve to teach how the sensors are built. Also it is clear to the skilled person that the spacers would in general be adjusted to fit specific problems, reflecting steric effects of the constituent components, binding affinities, the bias specific to a given hinge, and pH and salt conditions. The hinge sequences, spacer molecules, and other molecules depicted in examples 1-5 may each be individually selected as a potential component of the sensor molecule of the invention, or combinations thereof may be selected.

### SUMMARY

Resonance Energy Transfer (RET) based probes are widely used to understand spatio-temporal dynamics of protein pairs both in-vivo and in-vitro. It is well known that the choice of molecular linker connecting FP's in such probes can have a very strong effect on its overall performance. The approach taken here is to invent a radically new type of sensor by focusing on the structural properties required of the biased hinge mechanism to complement any give pair of sensor and ligand binding domain and associated pair of FP's and ligand of interest, thereby facilitating real time tracking of biochemical events, combined with strong signal and signal to noise characteristics. Our linker design is different in several key aspects from those devised hitherto, including flexible linkers of Matsuda et al.

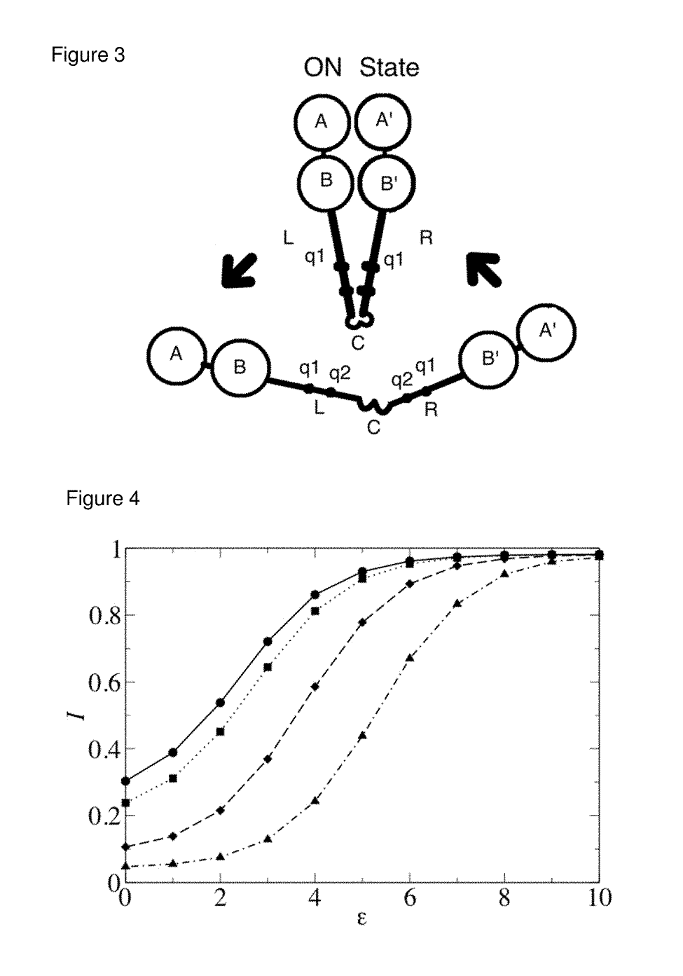

The mechanism can be understood as a radical change of the basic model of Komatsu et al, realizable when the flexible linker connecting the sensor and ligand binding domain is replaced by a biased hinge. The biased hinge in the latter context is designed to be in an open conformation (where the FP's are far apart and the FRET signal is low or negligible) in the OFF state see FIG. 2, and in the ON state oscillating between an open and closed conformation frequently enough to allow local concentration of analytes to remain close to endogenous levels see FIG. 3. In the rest of this document the sensor that results from this replacement will be referred to as a multistate dynamical unimolecular hinge sensor, or simply as the sensor in contexts when what is intended is clear.

An example of the unimolecular FRET or BRET sensor that is realizable using this biased hinge linker is drawn schematically in FIG. 2. The spheres represent macromolecules, interconnecting straight-lines denote rigid peptides labelled L and R respectively, while the curly element C denotes a highly flexible connected linker. The points labelled q1 and q2 denote charged or hydrophilic or hydrophobic residues (note the number 2 of such residues on L and on R is illustrative, there can be more and the number per peptide need not be the same). Not shown are short peptides acting as spacers between the macromolecules and linkers, or possible additional genetic sequences used for expression of the sensor in target organelles. The residues q1, q2, . . . and their locations are selected so that the probability of the hinge being open (i.e. the angle BCB′ approximately equals 180 degrees) in the ON state is approximately the same as being closed (i.e. the angle BCB' approximately 0 degrees). In one arrangement, the FP pair are depicted as macromolecules A and A′, and the ligand binding domain and sensor domain are depicted as macromolecules B and B′. In another, different arrangement, the FP pair can be depicted as macromolecules B and B′, and the ligand binding domain and sensor domain are depicted as macromolecules A and A′.

In the case that q1, q2, . . . are charged residues, their corresponding charges can be positive (arginine, histidine, lysine) or negative (asparatic and glutamic acid), for instance at a physiological pH corresponding to the selection of amino acid sequence.

The alpha-helical propensity of these molecules vary with arginine (0.21), histidine (0.61), lysine (0.26), asparatic Acid (0.69) and glutamic Acid (0.40) making histidine and asparatic acids possible choices (see reference NP14).

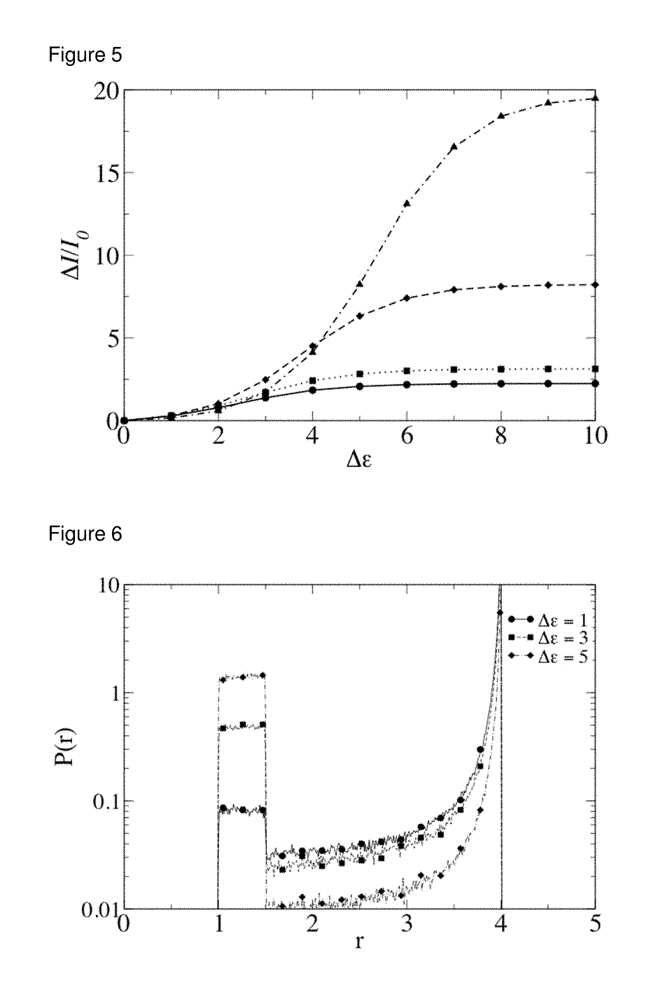

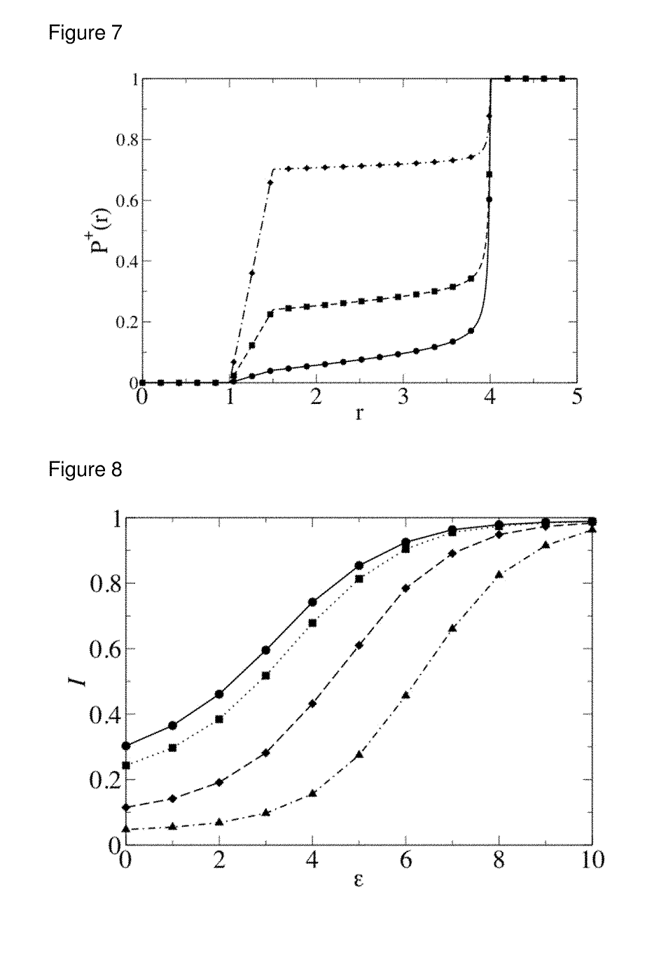

Test results obtained through Metropolis Monte Carlo simulation of an example of a unimolecular RET sensor (referred to as prototype 1) at physiological temperature (36° C.) are given in FIGS. 4-7 where the RET signal intensity, signal to noise ratio, and related probability distributions are displayed. The numerical model of prototype 1 consists of a potential V(r₁, r₂)=Vₛ(r₁, r₂)+V₁(r₁, r₂) where Vₛ(r₁, r₂) represents a switchable interaction between the ligand binding domain and sensor domain, and (r₁, r₂) are the position vectors of the idealized spheres modelling the ligand binding domain and donor FP, and the sensor domain and acceptor FP respectively, each of diameter G. In the OFF state, i.e. in the absence of the ligand or analyte Vₛ(r₁,r₂) ensures that the two spheres cannot overlap, which mathematically is implemented by the constraint that the distance r between the spheres is never less than σ, r>σ; in the ON state, it ensures that the two spheres do not overlap, but also experience a uniform attractive interaction of depth ε for σ<r≦σ+δ. The potential V₁(r₁, r₂)=Dₑ (1−exp(α(θ−180)))² models a biased hinge, using a Morse potential of depth Dₑ and inverse-width α, where θ is the angle between r₁ and r₂. The values of the parameters ε, σ, δ are generally selected to be close to the values of the real system of interest, for instance typically E is between 2 and 10 Kcal/mol, σ is ˜2.4 nm, δ is ˜1.5 nm to and a reasonable choice for Dₑ˜ε, and α−3.141/60.

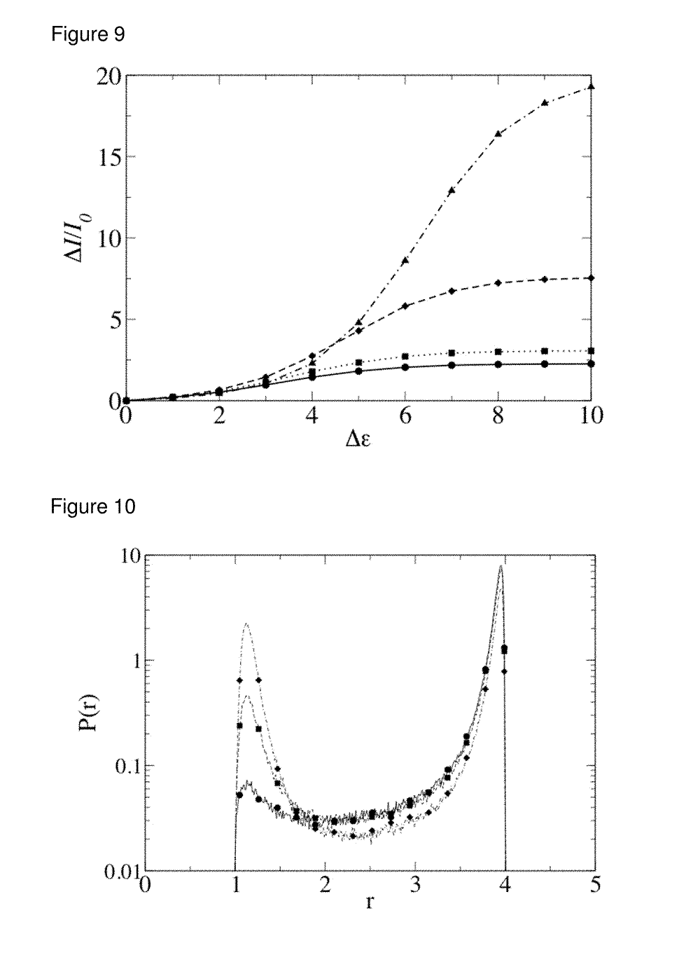

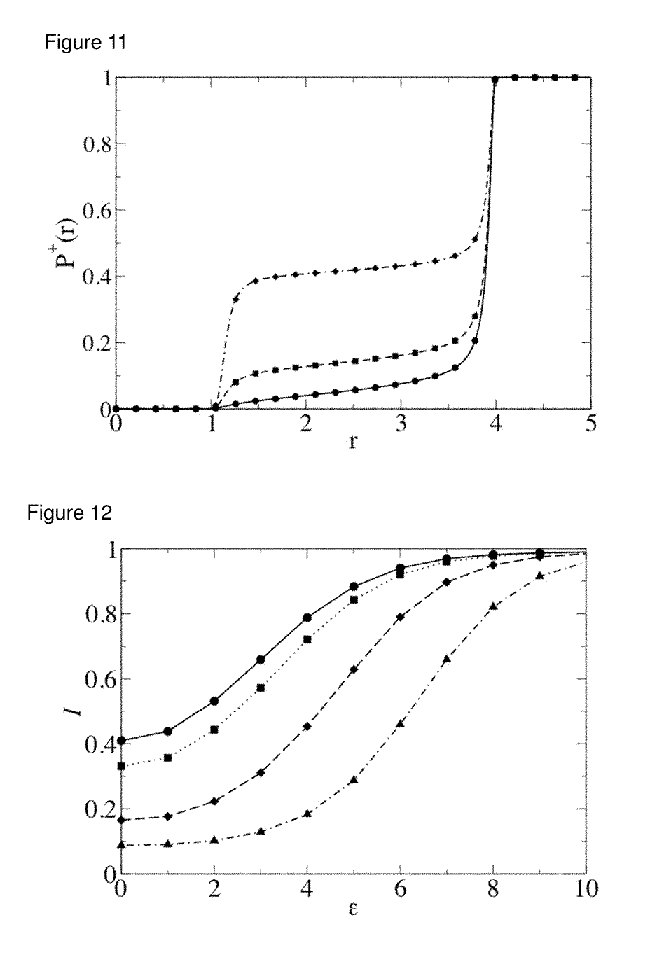

Prototype 2 is similar to prototype 1, except that in the ON state the attractive interaction of depth ε is replaced by a Lennard-Jones potential Vₛ (r₁, r₂)=4ε([σ/r]¹²−[σ/r]⁶). The test results are given in FIGS. 8-11 where the RET signal intensity, signal to noise ratio, and related probability distributions are displayed.

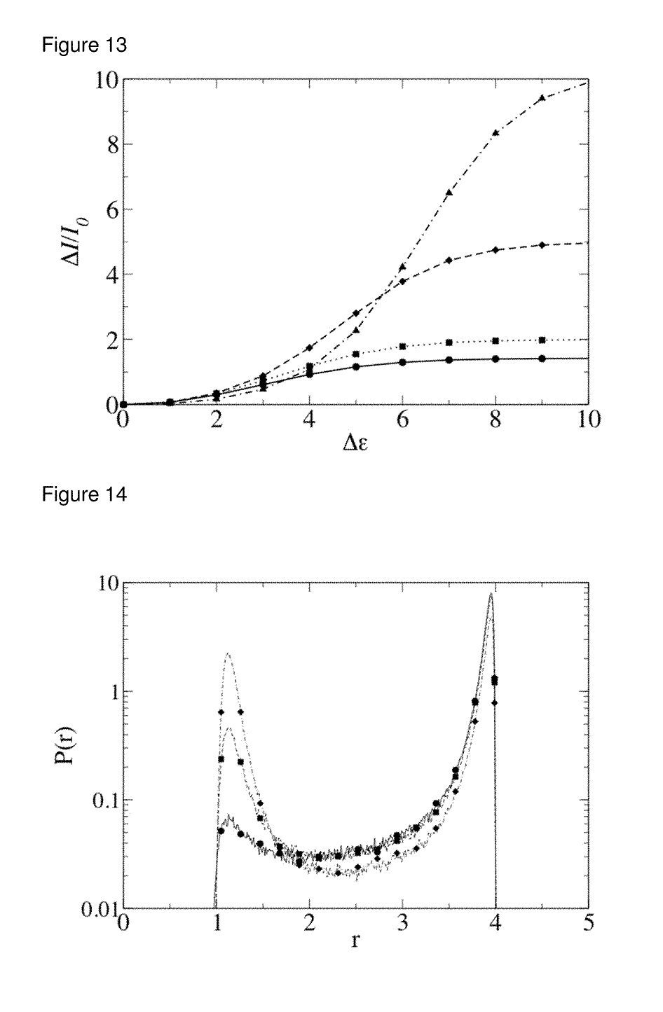

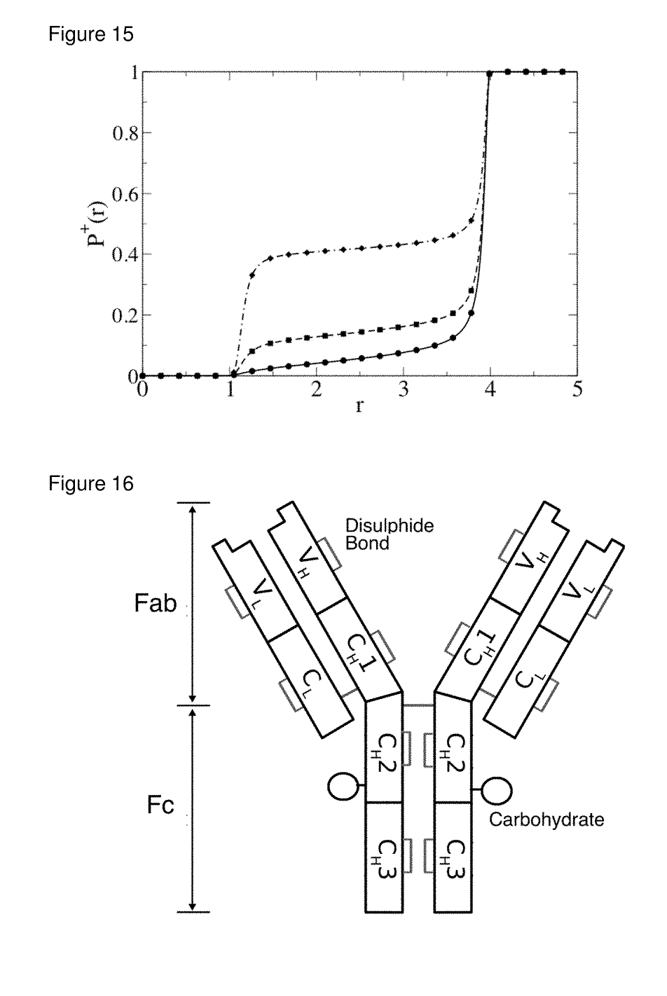

Prototype 3 is similar to prototype 1, except that in the OFF state Vₛ (r₁, r₂)=0.004 ([σ/r]¹²−[σ/r]⁶); and in the ON state 1 Vₛ (r₁, r₂)=4ε([σ/r]¹²−[σ/r]⁶). Qualitatively, the main difference between prototype 1 and prototype 3 is the use of a soft (continuous and differentiable interaction rather than a “hard core” interaction. The test results are given in FIGS. 12-15 where the RET signal intensity, signal to noise ratio, and related probability distributions are displayed. The test results are given in FIGS. 12-15 where the RET signal intensity, signal to noise ratio, and related probability distributions are displayed.

Unimolecular sensors having the structural and dynamical features depicted in FIGS. 2 and 3, can be readily generated. First, residue sequences of amino acids giving rise to stiff rod like peptides such as L or R are well known, and are widely available in the literature in the form of long alpha helical proteins such as Basic Leucine Zipper Domain (bZIP domain) found in many DNA binding proteins of almost eukaryotes. One example of bZIP, is a domain found in Maf transcription factor proteins NP15. Other long alpha helical structural motifs include coiled coils, examples include the muscle protein tropomyosin and oncoproteins c-Fos and c-jun (see reference NP16). Shorter alpha-helical motifs include the widely studied villin headpiece (see reference NP17).

Second, short very flexible peptides connecting the rods such as peptide C are also well known. Third, as mentioned above charged amino acids are also well known.

Fourth, the proteins which comprise the ligand binding domain, and sensor domains, and FP's can be taken from the literature (see reference NP1). Where estimates of the binding energy between particular ligand binding domains and sensor domains in the ON state in the presence of the ligand are not available, they can be estimated experimentally (see reference NP18), or computed via molecular simulation, using publically available standard force-fields developed for biology such as CHARMM (see reference NP19) or AMBER (see reference NP20), open source and publically available simulation engines such as NAMD (see reference NP21) or GROMACS (see reference NP22) and biased sampling methods such as those available in the open software package PLUMED, (see reference NP23) as well as commercial packages.

Once the binding energy is known (or estimated), the residues q1, q2, .. . in FIGS. 2 and 3 and their locations in the residue sequence defining the biased hinge can be optimised, via molecular simulation so that in the ON state the probability of the biased hinge sensor being open is slightly higher or equal to the probability of it being closed.

Another example of a biased hinge type sensor can be constructed where the curly element C in FIGS. 2 and 3 denotes a combination of rigid and flexible peptides rather than only a highly flexible peptide as the interconnecting linker. This example is by design more adaptable to chemical constraints associated with charged residues, and steric effects.

Having determined the full residue sequence of the full biosensor, the sensor can be generated using “off the shelf” biotechnology kits for example those made by: PURExpress® In Vitro Protein Synthesis Kit; Mammalian expression kits such as Jump In™ T-REx™ HEK 293 Kit; Cell-Free Expression Kits such as Expressway™ Maxi Cell-Free E. coli Expression System; and Bacterial expression kits such as Champion™ pET160 Directional TOPO® Expression Kit with LumioTM Technology.

Thus the linker can be tailor-made to match essentially any ligand binding domain and sensor domain, ligand and FP pair.

The ligand binding domain can be designed using various method such as Monoclonal Antibody, Polyclonal antibody or Genomic antibody technologies.

Macromolecules (for example FPs, ligand binding domains, sensor domains and even full unimolecular sensors) can be attached to specific sites of proteins of interest using chemical labelling for example covalent bonding amine labelling, thiol labelling etc. (see reference NP24), enzymatic labelling (labelling catalysed by post-translational enzyme modification, labelling with self-modified enzymes such as cutinase or interin, see reference NP25) and non-covalent tagging (tetracysteine/ biarsenical tag, histidine tag, see reference NP26). Other tags can be genetic based which include SNAP and CLIP tags (see reference NP27).

A practical issue in analytical chemistry, biochemistry, related sciences and industry is the perturbative effect of chemical sensors/indicators used to measure the concentrations of analytes of interest. If the sensor is not very sensitive to the target analyte, large volumes of probe may be required. Another frequent situation is that the design of the probe is such that it has a disruptive effect on the system it is designed to monitor, which complicates fine scale measurements, including the tracking of temporal and spatial variations of analyte concentrations. The present invention resolves both of these difficulties.

In parallel with developments of RET sensors using single donor and acceptor FPs, a method using a single FP donor but multiple FP acceptors (of different colours) has been reported, for instance by Sun et al, (see reference NP13). The latter method can be combined with the present invention, where for example the additional acceptor FPs are attached to sites of the protein of interest.

The present invention resolves many of the difficulties in performing immunoassays through the application of Tuneable Multistate Dynamical Unimolecular Hinge Sensors. In the context of immunoassays, the sensors have several novel capabilities, not possible or very difficult to implement with available methods. These include the facility to track in time the local concentrations of target analytes, to turn on pharmaceutically active molecules or toxins, and do not require the complex set of washing steps typically used with conventional immunoassays described above in the background art.

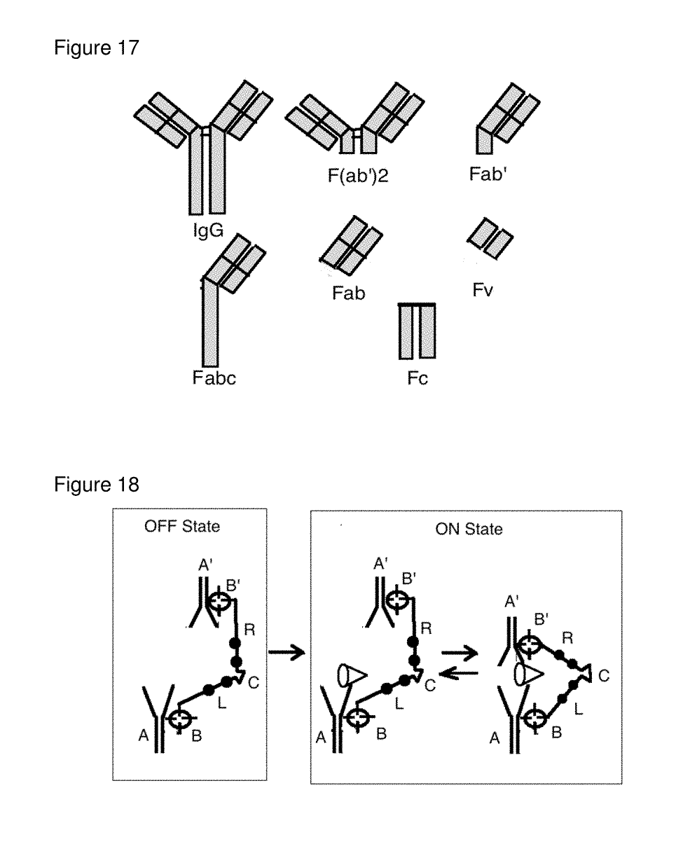

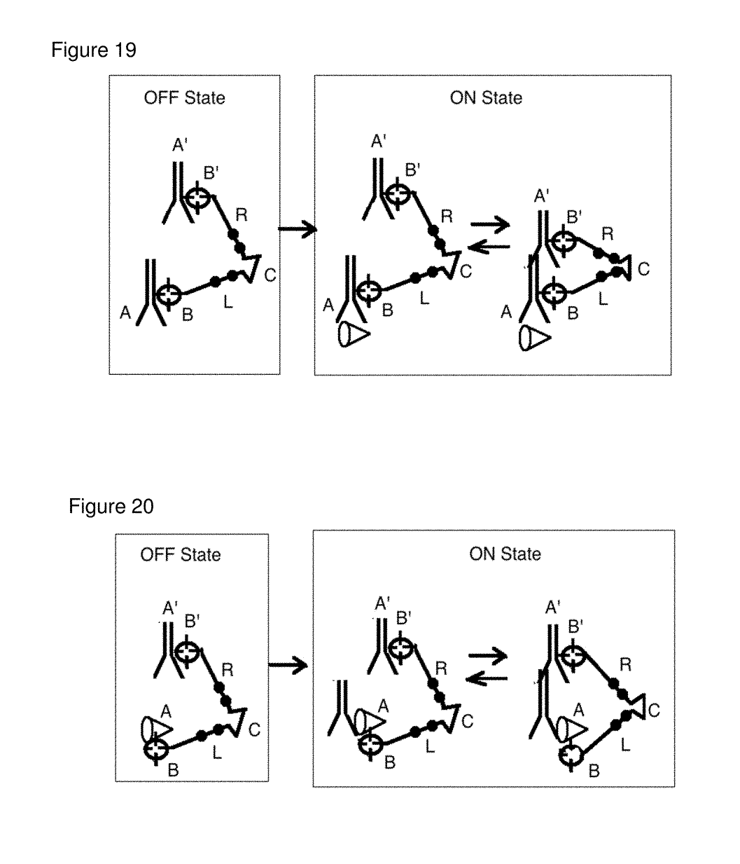

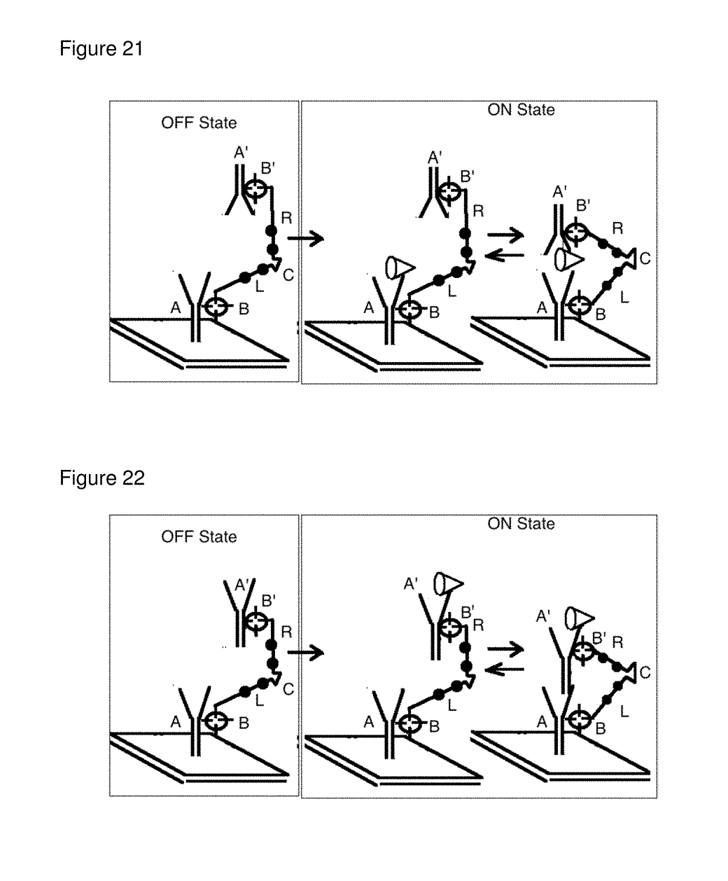

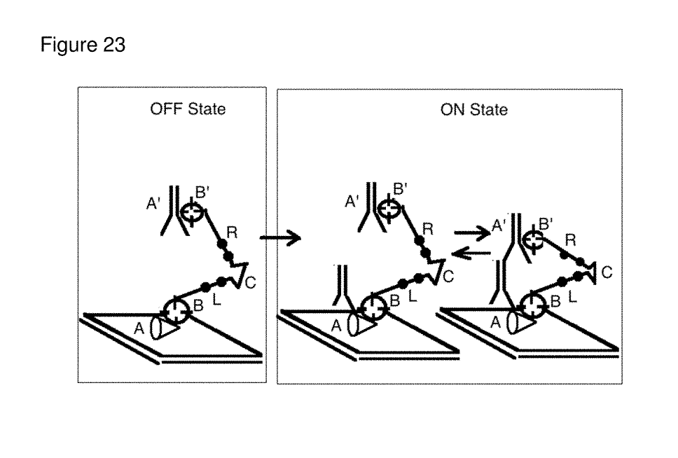

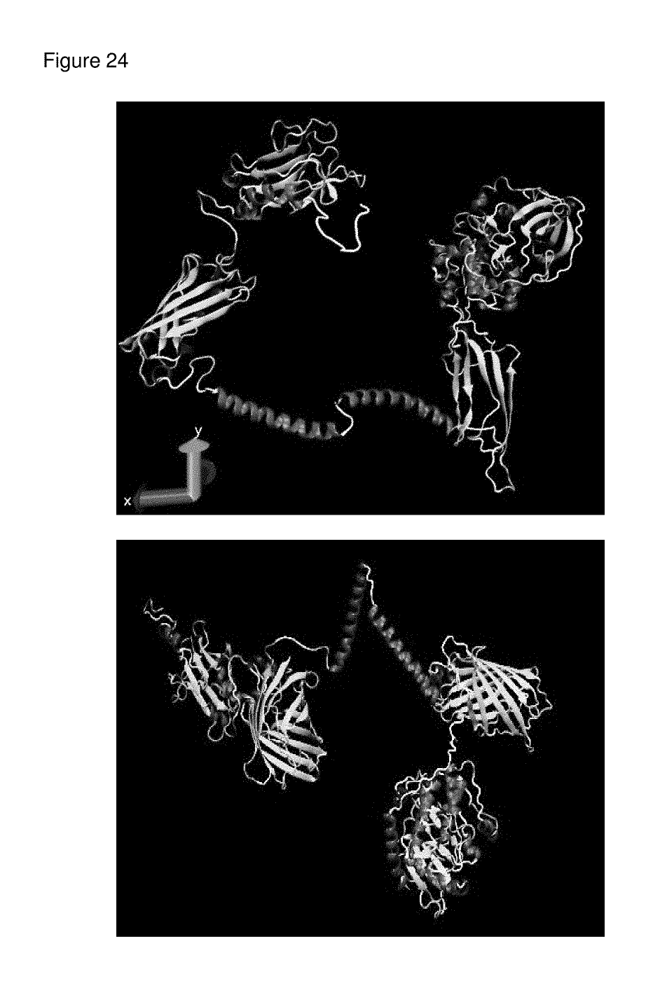

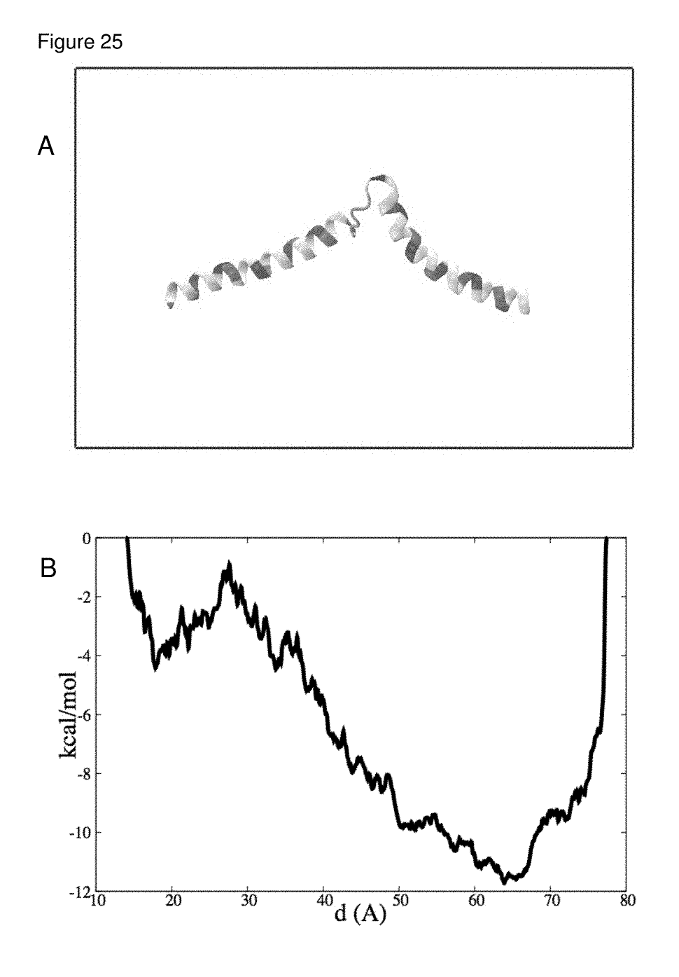

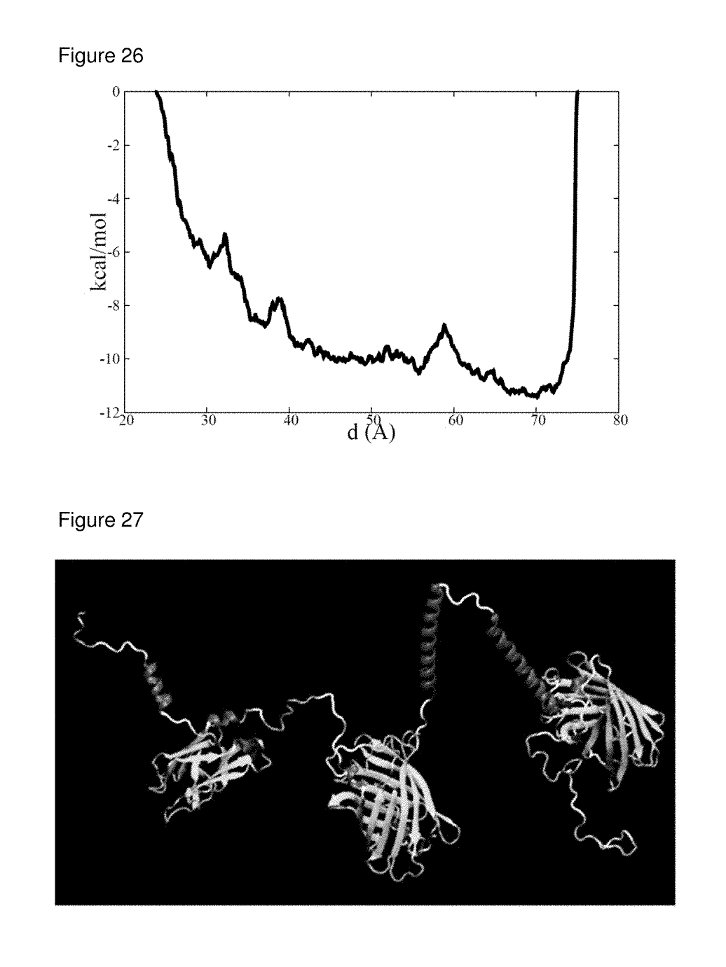

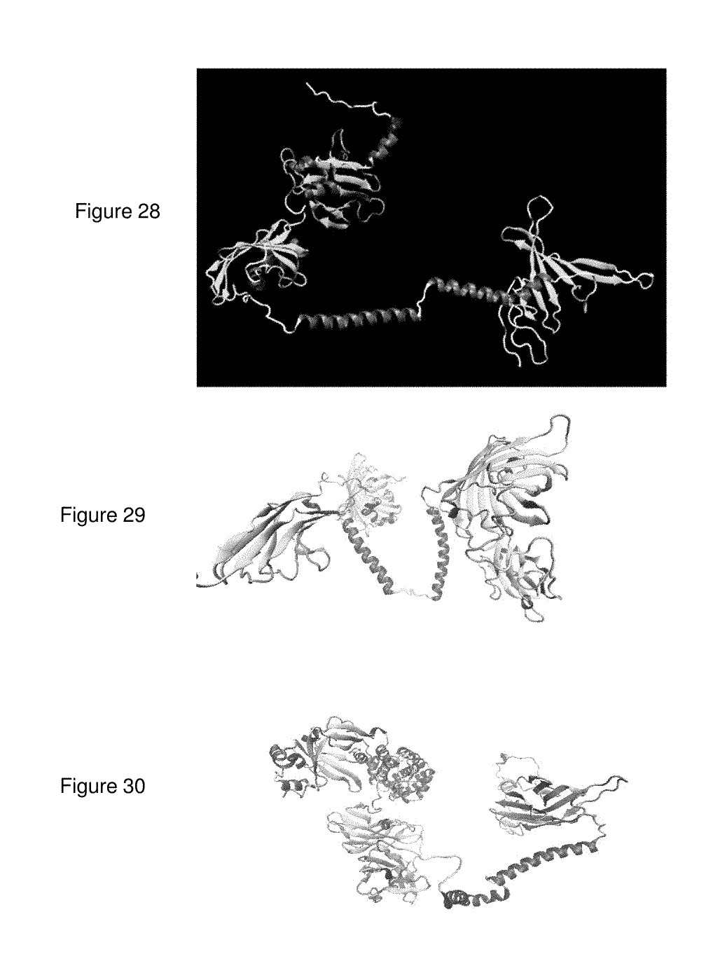

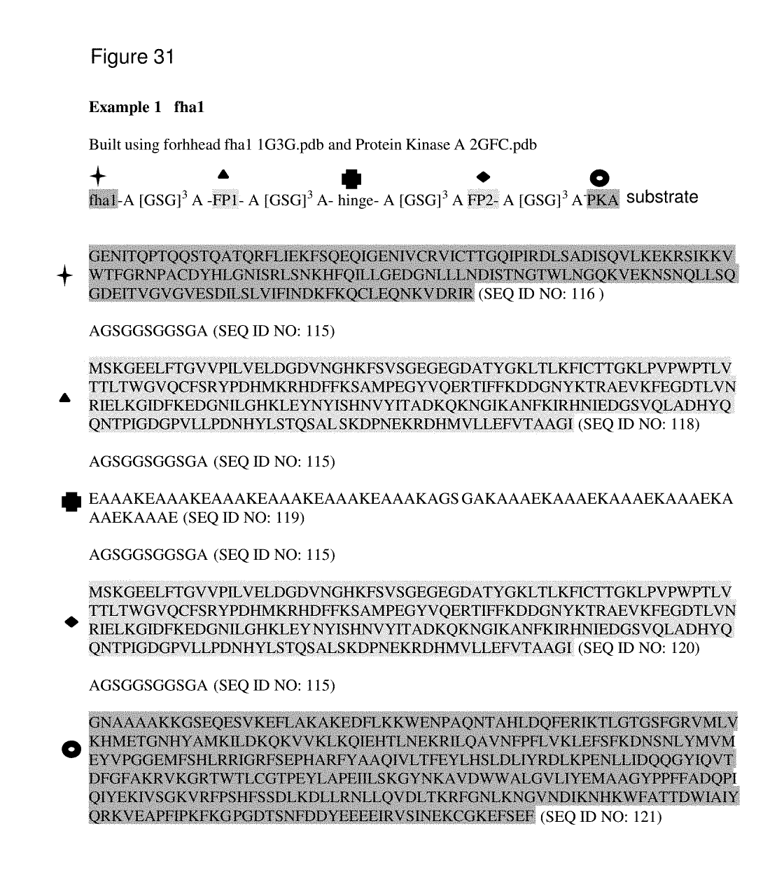

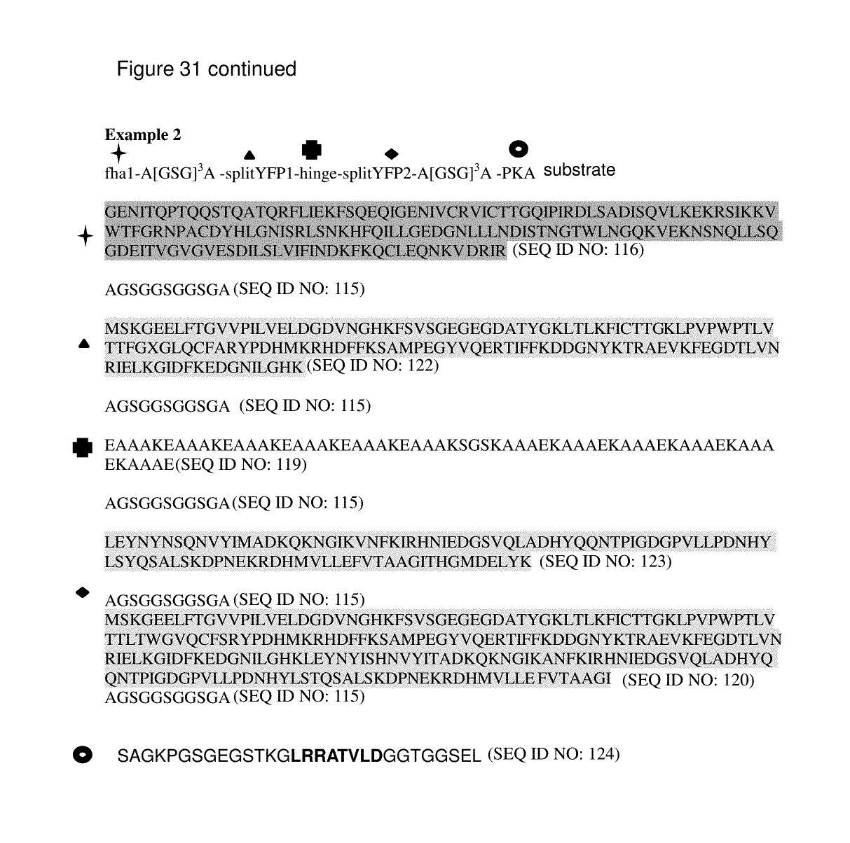

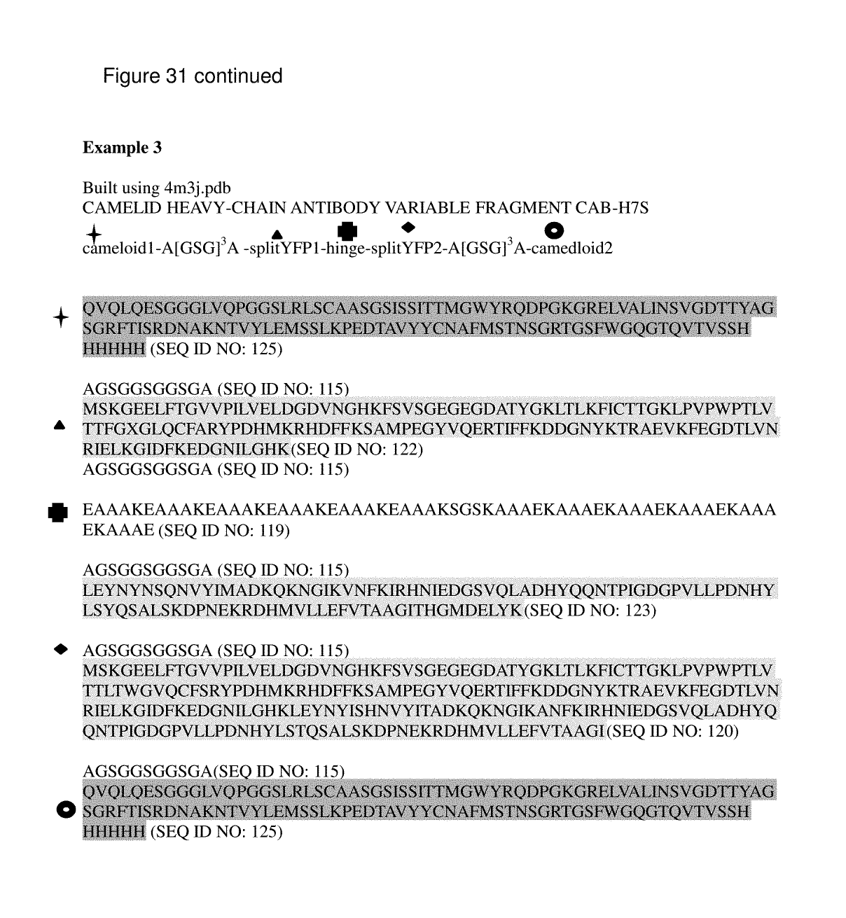

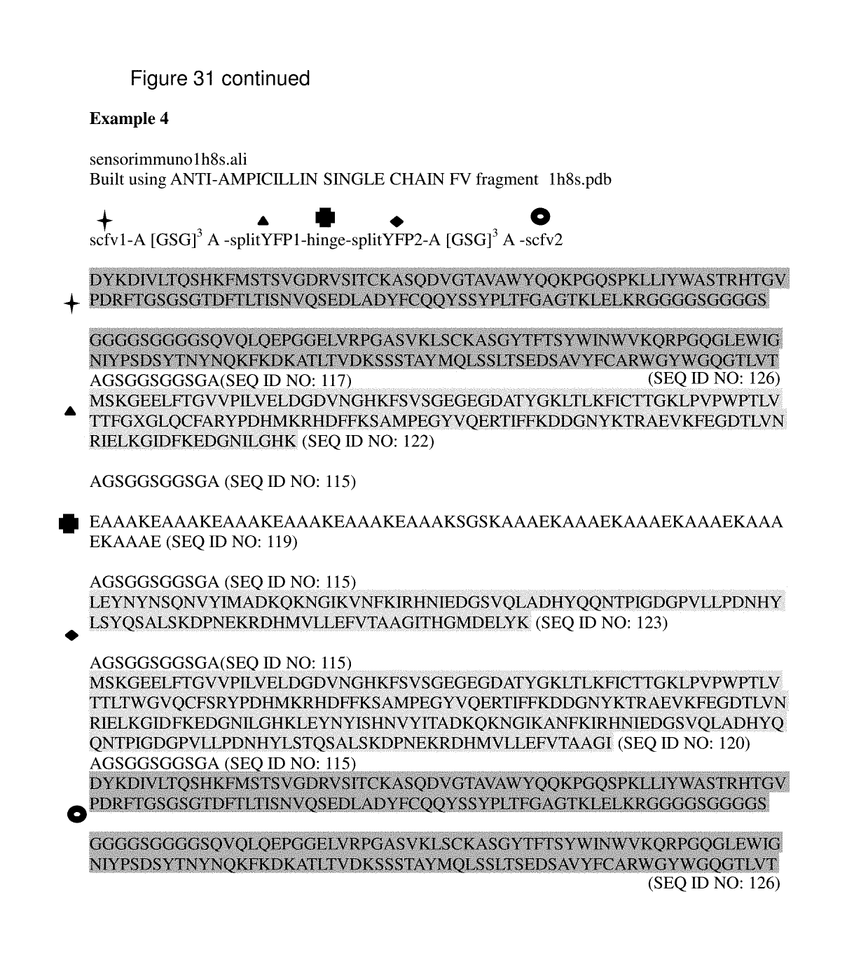

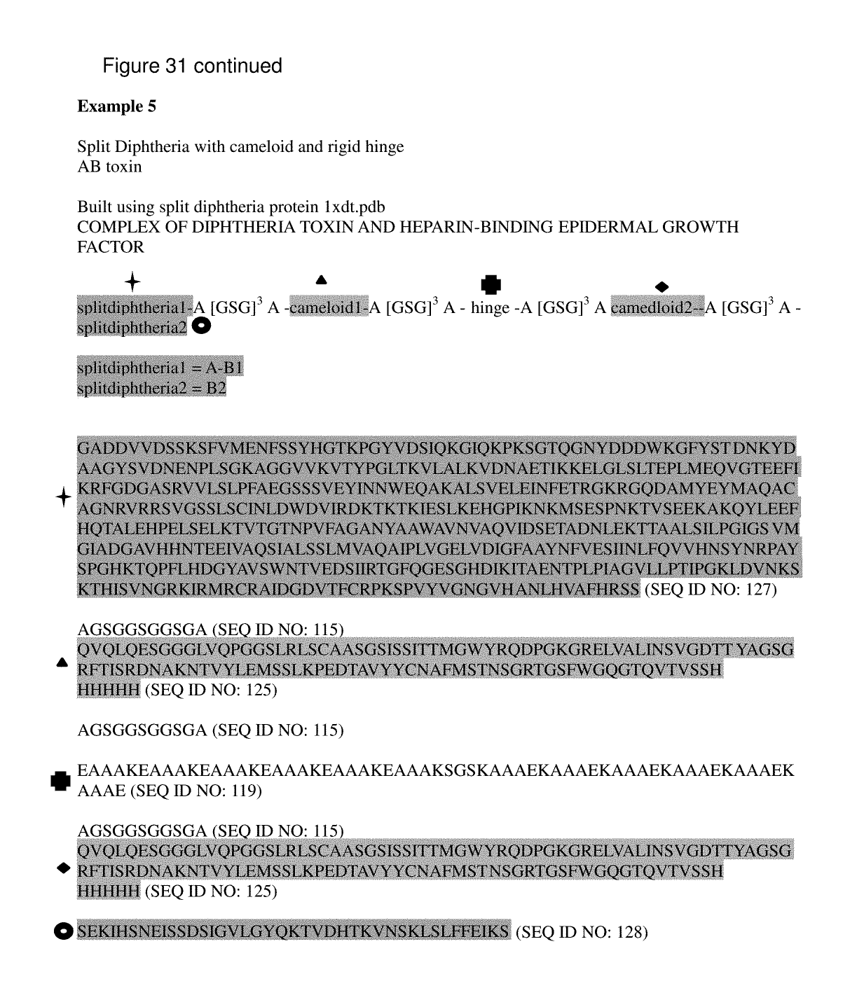

In this invention the detection of analytes such as antigens or antibodies (see FIG. 16) is made by selecting macromolecules A and A′ of the Tuneable Multistate Dynamical Unimolecular Hinge Sensors (see FIG. 2) to be primary antibodies or suitable antibody fragments (see FIG. 17). Macromolecules B and B′ can be a variety of different types of macromolecules interconnected through a biased hinge. In one version of this invention, A and A′ are selected to target different epitopes on the analyte, typically an antigen (see FIGS. 18 and 21). Alternatively, for cases where a second antibody binds to a corresponding primary antibody on the presence of the corresponding antigen, A is said primary antibody and A′ is the secondary antibody (see FIGS. 19 and 22). In another variant of this invention, A is an antigen, A′ is a secondary antibody, and the analyte is a corresponding primary antibody (see FIGS. 20 and 23. The sensor can be tuned so that in the OFF state (i.e. in the absence of target analytes close to A or A′), the arms of the hinge are open, and in the ON state (i.e. in the presence of target analytes close to A or A′), the arms of the hinge oscillate between open and closed configurations.

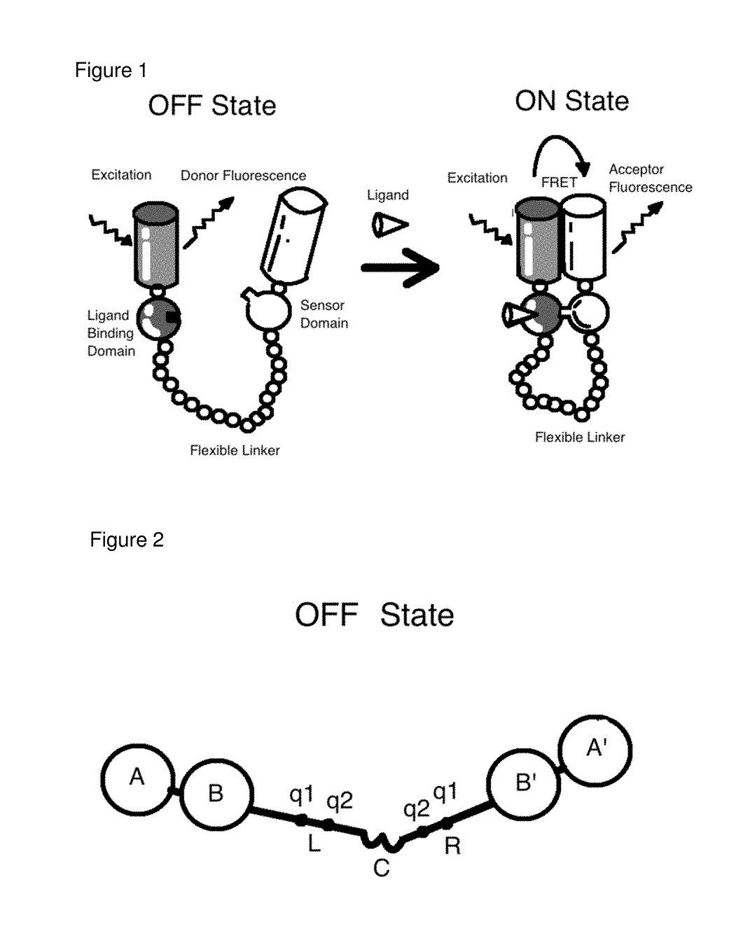

When A and A′ are primary antibodies (or primary antibody fragments containing accessible carbohydrates such as Fc or F(abc)) targeting different epitopes on the same antigen (which could be epitopes of the same type but at different locations), using the background art described above B can be conjugated with A through primary amines in the antibody, or through carbohydrates in the F_{C} region (see FIG. 16), and similarly B′ can be conjugated with A′ (with the use of blocking reagents as described above where required) as shown in FIG. 1.

When A and A′ are fragments of primary antibodies (e.g. F(ab′)₂, Fab′, Fab, Fv or F(abc)) targeting different epitopes on the same antigen (which could be epitopes of the same type but at different locations), A can be conjugated with B and A′ can be conjugated with B′ respectively using the sulfhydryl groups.

When A is a primary antibody (or primary antibody fragments containing accessible carbohydrates such as Fc or F(abc)), and A′ is a corresponding secondary antibody (or secondary antibody fragments containing accessible carbohydrates such as Fc or F(abc)), using the background art described above B can be conjugated with A through primary amines in the antibody, or through carbohydrates in the F_{C} region, and similarly B′ can be conjugated with A′ (with the use of blocking reagents as described above where required). It is also possible to conjugate A with B and A′ with B′ using the sulfhydryl groups described in the background art.

When A is an antigen and A′ is a secondary antibody (or fragment thereof), both targeting the same primary antibody or fragment thereof, appropriate labelling methods include the following. B can be conjugated with A through primary amines in the antibody, or through carbohydrates in the F_{C} region, and similarly B′ can be conjugated with A″(with the use of blocking reagents as described above where required). It is also possible to conjugate A with B and A′ with B′ using the sulfhydryl groups described in the background art.

In variant 1, and other aspects and embodiments, of this invention, macromolecules B and B′ are suitable fluorescent proteins or bioluminescent molecules, which can undergo resonance energy transfer when brought together by the action of A and A′ on the sensing of a target analyte, such as an antigen or antibody as described above.

In variant 2, and other aspects and embodiments, of this invention, B is a fluorescent protein or bioluminescent molecule or dye molecule capable of absorbing light from an external field and B′ to be a quencher macromolecule, for instance made from a metal such as gold, such that when A and A′ come together in their ON state, B and B′ to so that the energy absorbed by B is transferred non-radiatively to B′ and released locally in the form of heat on or close to the target analyte within or close to a cell or cellular compartment.

In variant 3, and other aspects and embodiments, of this invention, B or B′ are macromolecules which are the reactants of a chemical reaction such that when B and B′ are far apart the reaction cannot take place (in OFF state of A and A′), and when A and A′ come together in their ON state, the reaction can take place to produce products which may be pharmaceutical active or chemo-toxic. It is also possible that B consists of reactants which require a catalyst to react, and B′ consists of the corresponding catalyst, such that when A and A′ come together in their ON state, the resulting the products of the catalysed reaction are active drugs or chemo-toxic on or close to the target analyte within or close to a cell or cellular compartment.

In variant 4, and other aspects and embodiments, of this invention, B and B′ are each a moiety of a split single fluorophore (or split bioluminescent molecule) such that in the OFF state they are far apart and do not fluoresce (or bio-luminesce), and in the ON when A and A′ can come together, B and B′ are also brought together and fluoresce when illuminated at appropriate wavelengths (or bio-luminesce) on or close to the target analyte within or close to a cell or cellular compartment.

In variant 5, and other aspects and embodiments, of this invention, B and B′ are each the moieties of a split macromolecule which is pharmaceutically inactive (or non-chemo-toxic or non-photosensitizing) when they are far apart (in the OFF state of A and A′), and when A and A′ come together in their ON state due to the presence of the target analyte, B and B′ are brought together so that the complex is pharmaceutical active (or chemo-toxic or photosensitizing).

Variant 6 of this invention is a combination of variant 4 and variant 5. B consists of a moiety of a split fluorescent protein or split bioluminescent molecule and the moiety of a split drug or split chemo-toxic or split photosensitizing molecule macromolecule, and B′ consists of the other moiety of the split fluorescent protein or split bioluminescent molecule and the other moiety of a split drug or split chemo-toxic molecule, such that when B and B′ are apart the complex is inactive, and when B and B′ are brought together due to the presence of target analyte by A and A′, the complex becomes pharmaceutically active or chemo-toxic or photosensitizing and fluorescent or bioluminescent.

In variant 7, and other aspects and embodiments, of this invention, B consists of the moieties of a split fluorescent protein or split bioluminescent molecule and a quencher molecule, and B′ consist of the other moieties of the split fluorescent protein or split bioluminescent molecule and a quencher molecule, such that B and B′ are apart they cannot become easily optically excited, for instance by an external field, but when B and B′ are brought together due to the recognition of a target analyte by A and A′, the resulting complex both easily absorbs energy from an externally applied source and transfers it non-radiatively through the quencher molecule, thereby heating the local cellular or sub-cellular region wherein the analyte is located. It is also possible to divide the quencher into two moieties, with one quencher moiety and one fluorescent moiety in B, and one quencher moiety and one fluorescent moiety in B′.

Variant 8, and other aspects and embodiments, of this invention is a combination of variants 3 and 4. B consists of the moieties of a split fluorescent protein or split bioluminescent molecule and some of the reactants of a chemical reaction and B′ consist of the other moieties of the split fluorescent protein or split bioluminescent molecule and the rest of the reactants required for a chemical reaction. It is also possible that B consists of reactants which require a catalyst to react, and and B′ consists of the corresponding catalyst. When B and B′ are apart the they cannot easily fluoresce or bioluminesce, and the chemical reaction cannot easily take place, but when B and B′ are brought together due to the recognition of a target analyte by A and A′, the resulting complex can fluoresce or bioluminesce, and the chemical reaction can take place at or close to the local cellular or sub-cellular region wherein the analyte is located.

As well as sensors targeting single analytes in the above variants of the invention, multiple sensors targeting different analytes, each using corresponding macromolecules (A,A′,B and B′) can be used at the same time within a living sample or microtiter testing well or vial. When appropriate, different acceptor fluorophores (or moieties thereof) emitting at different wavelengths can be used so as to allow simultaneous use and/or measurement (using microscopy) of each type of possible analyte present in the sample.

Variants 1, 4, 6 and 8, and other aspects and embodiments, of this invention can be used for performing immunoassays to identify the presence of analytes including antibodies in samples using microscopy and suitable light sources for the selected fluorescent proteins, or no external light sources if the donor fluorescent protein is bioluminescent or chemiluminescent.

Variants 1, 4, 6 and 8, and other aspects and embodiments, of this invention, when combined with confocal scanning microscopy described in the background art, can be used to identify the time dependent concentration and location of analytes in a sample.

This includes the capacity to generate three dimensional spatial images of the concentration of analytes and track their position over time, including in living cells. The invention can also be used to monitor changes in real time in such analyte concentration through the use of appropriate flow chambers, or in living cells.

Desktop scanners or an ordinary CCD camera, and either a single LED of a single wavelength or a combination of Red-Green-Blue LEDs can be combined with variants 1, 4, 6 and 8, and other aspects and embodiments, of this invention to determine the concentration of the analyte in a sample, including its time dependence, to produce multi-dimensional images tracking over time the concentration of the analyte.

Variants 1, 4, 6 and 8, and other aspects and embodiments, of this invention can be combined with either a single LED of a single wavelength or a combination of Red-Green-Blue LEDs and photomultiplier diode chips to measure the photo emission of the sensor, and thereby determine the concentration of the analyte in a sample, including its time dependence, for instance when a three or two dimensional image is not required. This can be used for taking and analysing immunoassays in the field, as well as in specialised laboratories.

When A or A′ is bonded to a suitable surface of a flow chamber, variants 1,4,6 and 8, and other aspects and embodiments, of this invention can be used to measure the time dependent concentration of analytes, including their detection in continuous sample measurement (see FIGS. 21,22, and 23).

When A or A′ is bonded to a suitable surface of a suitable titration vessel, the invention can be used for measuring the concentration of one or multiple analytes during a titration.

When macromolecules B and B′ are the moieties of a split bio-luminescent or chemiluminescent molecule, the invention can be combined with low cost microtiter and vials described in the background art, to perform and analyse immunoassays in the field i.e. in non-laboratory conditions and without the use of specialised equipment.

When macromolecules B and B′ are the moieties of a split bio-luminescent or chemiluminescent molecule, the invention can be used to deliver light close to the location of analytes, which can be close to or within living cells or cellular compartments, for the purpose of targeted electromagnetic radiation treatment.

When macromolecules B and B′ are pairs of chemical reactants which react spontaneously when brought together, and such that the chemical product is either a drug or a chemo-toxin or a chemo-toxin or photosensitizing, the invention can be used as a therapeutic for diseased living cells or sub-cellular compartments, and such that the drug or chemo-toxin becomes activated on the presence of a target analyte. If B also includes either a donor FP or bioluminescent molecule or a moiety of a split FP or a moiety of a split bioluminescent molecule and B′ includes a corresponding acceptor FP, or corresponding moiety of a split FP or corresponding moiety of a split bioluminescent, the activation of a drug or chemo-toxin or photosensitizing agent can be marked by the emission of light. When a donor fluorophore is used an external light source is required. When either a non-split fluorophore or non-split bioluminescent molecule is used as a donor, the emission signalling recognition of the analyte is due to resonance energy transfer.

When B is a fluorophore or a chromophore and B′ is a quencher, and given a light source, variants 2 and 7, and other aspects and embodiments, of the invention can be used to deliver non-radiative energy in the form of heat at or close to the location of target analytes in living cells.

### Discussion

It is well known that the choice of molecular linker connecting FP's in the probe can have a very strong effect on its overall performance. The approach taken here is to invent a radically new type of sensor by focusing on the structural properties required of the linker mechanism to complement any given pair of sensor and ligand binding domain and associated pair of FP's and ligand of interest, thereby facilitating real time tracking of biochemical events, combined with strong signal and signal to noise characteristics. Our biased hinge design and the resulting sensor is different in several key aspects from the one described in references PT1 and NP11.

The highly tuneable multistate dynamical hinge sensor is designed (i.e. biased) to be normally fully open in OFF state (i.e. the absence of the target ligand) so as to ensure the FP pair are far apart and the corresponding RET rate is very low. The sensor is tuned so that when combined with a ligand binding domain and sensor domain and associated FP's, it can open and close frequently in the ON state, but in such a way that it is can be selected to be on average open more often than closed.

This intrinsic fluctuating feature in the ON state accounts for the high signal and signal to noise properties, while allowing concentrations levels of target analyte to be maintained close to endogenous levels. This design feature of choosing the ON state to be fluctuating between two conformations (open and closed) rather than simply closed, is completely counter intuitive, and novel.

The sensor described above need not be protein based, its components can be organic or inorganic or a mixture thereof.

The present invention in the context of immunoassays also creates a series of novel capabilities not possible or very difficult to implement with available methods. In this invention the detection of analytes such as antigens is made through macromolecules A and A′ of the Tuneable Multistate Dynamical Unimolecular Hinge Sensors (see FIG. 2). Macromolecules A and A′ can be selected to each be primary antibodies targeting different epitopes on the analyte, which may the same type of epitope but at different locations (see FIGS. 18 and 21). Alternatively, for cases where a second antibody binds to a corresponding primary antibody only the presence of the corresponding antigen, A is said primary antibody and A′ is the secondary antibody (see FIGS. 19 and 22). In another variant of this invention, A is an antigen, A′ is a secondary antibody, and the analyte is a corresponding primary antibody (see FIGS. 20 and 23). The sensor can be tuned so that in the OFF state (i.e. in the absence of target analytes close to A or A′), the arms of the hinge are open, and in the ON state (i.e. in the presence of target analytes close to A or A′), the arms of the hinge oscillate between open and closed configurations. Macromolecules B and B′ can each consist of one or more selected molecules or moieties of split molecules, which when brought close together due to the presence of target analytes can produce a variety of selected effects: (a) resonance energy transfer in the presence of a suitable electro-magnetic radiation field; (b) fluorescence in the presence of a suitable electro-magnetic radiation field; (c) bioluminescence; (d) activated drug; (e) activated chemo toxin; (f) chemical reaction; (g) catalysed chemical reaction; (h) in the presence of a suitable electro-magnetic radiation field, the release of heat through quenching; (i) in the presence of a suitable electro-magnetic radiation field either of an external or endogenous source, the production of reactive oxygen. In addition several of these effects can be combined in the same sensor, including: {C1 (a-c), C2 (a-d), C3 (a-e), C4 (a-f), C5 (a-g), C6 (a-h); C7 (a-i), C8 (b-d), C9 (b-e), C10 (b-f), C11 (b-g), C12 (b-h), C13 (b-i), C14 (c-d), C15 (c-e), C16 (c-f), C17 (c-g), C17 (c-h), C18 (c-i)}. These effects take place in the vicinity on the sensor, which can be close to or within cells, cellular compartments, or in vitro.

Variants of the invention can be used to visualise and track in time analytes in vivo and in vitro in assays to create multi-dimensional images of said analytes using confocal scanning microscopy, desktop scanners, a variety of suitable LEDS and photo cascade chips. In addition variants of the invention can be deployed in the field using microtiters or vials, without specialised equipment, and at low cost for several applications including environmental, health, and food safety. Multiple sensors targeting different analytes can be deployed to measure a single sample simultaneously, and can be used to measure the time dependent concentration of analytes in suitable flow chambers or through titration.

Variants of the invention can be used to deliver payloads to regions close to and within cellular environments which can be specified if required by genetic targeting, and such that the payloads become activated on the presence of target analytes, and are inactive in their absence. The payloads can include drugs, chemo-toxins, chemicals, catalysts, heat through the localised absorption of external electro-magnetic or chemical fields, and hydrogen radicals using photosensitizers coupled with electro-magnetic fields of external or endogenous origin.

The hinge of the highly tuneable multistate dynamical unimolecular hinge sensor is designed (i.e., biased) to be normally fully open in OFF state (i.e. the absence of the target ligand) so as to ensure the FP pair are far apart and the corresponding RET rate is very low. The biased hinge is tuned so that when combined with a ligand binding domain and sensor domain and associated FP's, it can open and close frequently in the ON state, but in such a way that it is can be selected to be on the average open more often than closed. This intrinsic fluctuating feature in the ON state accounts for the high signal and signal to noise properties, while allowing concentrations levels of target analyte to be maintained close to endogenous levels. This design feature of choosing the ON state to be fluctuating between two configurations (open and closed) rather than simply closed, is completely counter intuitive, and novel. A version of this sensor including inorganic components can be used in electronic, semi-conducting and quantum computing industries as electronic sensors, memory devices and nano-actuators. The present invention in the context of immunoassays also creates a series of novel capabilities not possible or very difficult to implement with available methods. Variants of the invention can be used to deliver payloads to regions close to and within cellular environments which can be specified if required by genetic targeting, and such that the payloads become activated on the presence of target analytes, and are inactive in their absence. The payloads can include drugs, chemo-toxins, chemicals, catalysts, heat through the localised absorption of external optical or chemical fields, and oxygen radicals as used in photodynamic therapy.

### EXAMPLE SEQUENCES

fha1-fp1-hinge-fp2-pka substrate
(SEQ ID NO: 1)
The protein sequence (single letter) is
GENITQPTQQSTQATQRFLIEKFSQEQIGENIVCRVICTTGQIPIRDLSA
DISQVLKEKRSIKKVWTFGRNPACDYHLGNISRLSNKHFQILLGEDGNLL
LNDISTNGTWLNGQKVEKNSNQLLSQGDEITVGVGVESDILSLVIFINDK
FKQCLEQNKVDRIRAGSGGSGGSGAMSKGEELFTGVVPILVELDGDVNGH
KFSVSGEGEGDATYGKLTLKFICTTGKLPVPWPTLVTTLVQCFSRYPDHM
KRHDFFKSAMPEGYVQERTIFFKDDGNYKTRAEVKFEGDTLVNRIELKGI
DFKEDGNILGHKLEYNYISHNVYITADKQKNGIKANFKIRHNIEDGSVQL
ADHYQQNTPIGDGPVLLPDNHYLSTQSALSKDPNEKRDHMVLLEFVTAAG
IAGSGGSGGSGAEAAAKEAAAKEAAAKEAAAKEAAAKEAAAKAGSGAKAA
AEKAAAEKAAAEKAAAEKAAAEKAAAEAGSGGSGGSGAMSKGEELFTGVV
PILVELDGDVNGHKFSVSGEGEGDATYGKLTLKFICTTGKLPVPWPTLVT
TFLQCFARYPDHMKRHDFFKSAMPEGYVQERTIFFKDDGNYKTRAEVKFE
GDTLVNRIELKGIDFKEDGNILGHKLEYNYNSQNVYIMADKQKNGIKVNF
KIRHNIEDGSVQLADHYQQNTPIGDGPVLLPDNHYLSYQSALSKDPNEKR
DHMVLLEFVTAAGIAGSGASAGKPGSGEGSTKGLRRATVLDGGTGGSEL
fha1-splitYFP1 -hinge-splitYFP2-pka substrate
(SEQ ID NO: 2)
The protein sequence (single letter) is
GENITQPTQQSTQATQRFLIEKFSQEQIGENIVCRVICTTGQIPIRDLSA
DISQVLKEKRSIKKVWTFGRNPACDYHLGNISRLSNKHFQILLGEDGNLL
LNDISTNGTWLNGQKVEKNSNQLLSQGDEITVGVGVESDILSLVIFINDK
FKQCLEQNKVDRIRAGSGGSGGSGAMSKGEELFTGVVPILVELDGDVNGH
KFSVSGEGEGDATYGKLTLKFICTTGKLPVPWPTLVTTFGXGLQCFARYP
DHMKRHDFFKSAMPEGYVQERTIFFKDDGNYKTRAEVKFEGDTLVNRIEL
KGIDFKEDGNILGHKAGSGGSGGSGAEAAAKEAAAKEAAAKEAAAKEAAA
KEAAAKSGSKAAAEKAAAEKAAAEKAAAEKAAAEKAAAEAGSGGSGGSGA
LEYNYNSQNVYIMADKQKNGIKVNFKIRHNIEDGSVQLADHYQQNTPIGD
GPVLLPDNHYLSYQSALSKDPNEKRDHMVLLEFVTAAGITHGMDELYKAG
SGASAGKPGSGEGSTKGLRRATVLDGGTGGSEL

### Antibody Binding Affinities

TABLE 1
K_{D} (the equilibrium dissociation constant between the antibody and its
antigen), of 840 Rabbit Monoclonal Antibodies (RabbitMAbs) and 88
MouseAbs expressed as percentage distribution at a given binding
affinity from micromolar to femtomolar range.

| K_{D} Value | RabbitAbs | MouseAbs |
|---|---|---|
| >10⁻⁷ | 6% | |
| 10⁻⁷ | 11% | |
| 10⁻⁸ | 19% | 1% |
| 10⁻⁹ | 39% | 1% |
| 10⁻¹⁰ | 10% | 35% |
| 10⁻¹¹ | 13% | 54% |
| 10⁻¹² | 2% | 8% |
| 10⁻¹³ | | 1% |

K_{D} values for 88 MouseAbs were derived from published literature. The K_{D} measurement values for the 863 RabbitMAbs were all from the OI-RD measurements. RabbitMAbs appear to be on average 1-2 order of magnitude higher affinity. Origin of data—http://www.abcam.com/index.html?pageconfig=resource&rid=15749

| K_{D} value | Molar concentration (sensitivity) |
|---|---|
| 10⁻⁴ to 10⁻⁶ | Micromolar (uM) |
| 10⁻⁷ to 10⁻⁹ | Nanomolar (nM) |
| 10⁻¹⁰ to 10⁻¹² | Picomolar (pM) |
| 10⁻¹³ to 10⁻¹⁵ | Femtomolar (fM) |

### REFERENCES

References discussed herein are incorporated by reference.

Patents and Published Patent Applications

PT1 EP 2623514 A (KYOTO UNIVERSITY) 26.09.2011

PT2 Wang. X. Optical systems for microarray scanning. U.S. Pat. No. 7,706,419 B2. (Apr. 7, 2010)

### Non-Patent Literature

NP1 NEWMAN, Robert, et al. Genetically Encodable Fluorescent Biosensors for Tracking Signaling. Chemical Reviews. 2011, vol. 111, no. 5, p 3614-3666.

NP2 MIYAWAKI, Atsushi. Visualization of Spatial and Temporal Dynamics on Intracellular Signaling. Developmental Cell. 2003, vol. 4, n. 3, p. 295-305.

NP3 MIYAWAKI, Atsushi. Cellular Functions Using Fluorescent Proteins and Fluorescence Resonance Energy Transfer. Annual Review of Biochemistry. 2011, vol. 80, p. 357-373.

NP4 FRITZ, Rafael, et al. A Versatile Toolkit to Produce Sensitive FRET Biosensors to Visualize Signaling in Time and Space. Science 2013, vol 6, no. 285, p. 1-13.

NP5 LAKOWICZ, Joseph. Principles of Fluorescent Spectroscopy. USA: Springer, 2010. ISBN 0387312781. p. 954.

NP6 MIYAWAKI, Atsushi, et al. Fluorescent indicators for Ca2+ based on green fluorescent proteins and calmodulin. Nature. 1997, vol. 388, no. 6645, p 882-7.

NP7 ALLEN, Michael, et al. Subcellular dynamics of protein kinase activity visualized by FRET-based reporters, 2006, Biochemical and bio-physical research communications. 2006, vol. 348, no. 2, p. 716-721.

NP8 ZHANG, Jin, et al. FRET-based biosensors for protein kinases: illuminating the kinome. Mol. BioSyst. 2007, vol. 3, no. 11, p. 759-765.

NP9 LISSANDRON, Valentina, et al. Improvement of a fret-based indicator for camp by linker design and stabilization of donor-acceptor interaction. J. Mol. Biol. 2005, vol 354, no. 3, p. 546-55.

NP10 PERTZ, Oliver, et al. Spatiotemporal dynamics of rhoa activity in migrating cells. Nature. 2006, vol. 440, p. 1069-1072.

NP11 KOMATSU, Naoki, et al. A Development of an optimized backbone of FRET biosensors for kinases and GTPases. Molecular Biology of the Cell. 2011, vol. 22, p. 4647-4658.

NP12 Palmer, Amy, et al. Ca2+ indicators based on computationally redesigned calmodulin-peptide pairs. Chem. Biol. 2006, vol. 13, p. 521.

NP13 SUN, Yuansheng, et al. Three-Color Spectral FRET Microscopy Localizes Three Interacting Proteins in Living. Biophys J. 2010, vol. 99, no. 4, p. 1274-1283.

NP14 PACE, C, et al. A Helix Propensity Scale Based on Experimental Studies of Peptides and Proteins. Biophys J. 2010, vol. 75, no. 1, p. 422-427.

NP15 KUSUNOKI, Hideki, et al. Solution structure of the DNA-binding domain of MafG. Nat. Struct. Biol. 2010, vol. 9, no. 4, p. 252-256.

NP16 Mason, Jody, et al. Coiled coil domains: stability, specificity, and biological implications. Chembiochem. 2004, vol. 5, no. 2, p. 170-176.

NP17 RIPOLL, Daniel, et al. Folding of the villin headpiece subdomain from random structures. Analysis of the charge distribution as a function of pH. J Mol Biol. 2004, vol. 339, no. 4, p. 915-925.

NP18 VREVEN, Thom, et al. Prediction of protein-protein binding free energies. Protein Sci. 2012, vol. 21, no. 3, p. 396-404.

NP19 MACKERELL, Alex, et al. All-atom empirical potential for molecular modeling and dynamics studies of proteins. Journal of Physical Chemistry B. 1998, vol. 102, p. 3586-3616.

NP20 LINDORFF-LARSEN, Kresten, et al. Improved side-chain torsion potentials for the Amber ff99SB protein force field. Protein Sci. 2010, vol. 78, no. 8, p. 1950-1958.

NP21 PHILLIPS, James, et al. Spatiotemporal dynamics of rhoa activity in migrating cells. Journal of Computational Chemistry. 2005, vol. 26, p. 1781-1802.

NP22 PRONK, Sander, et al. GROMACS 4.5: a high-throughput and highly parallel open source molecular simulation toolkit. Bioinformatcs. 2012, vol. 29, no. 7, p. 845-854.

NP23 Bonomi, Massimiliano, et al. PLUMED: A portable plugin for free-energy calculations with molecular dynamics. Comp. Phys. Comm. 2009, vol. 180, no 10, p. 1961-1972.

NP24 LOMANT, A, et al. Chemical probes of extended biological structures: Synthesis and properties of the cleavable protein cross-linking reagent [35S] dithiobis (succinimidyl propionate) J. Mol. Biol. 1976, vol. 104, no. 1, p. 243-261.

NP25 X U Ming-qun, et al. In vitro protein splicing of purified precursor and the identification of a branched intermediate. Cell. 1993, vol. 75, no. 7, p. 1371-1377.

NP26 OJIDA, Akido, et al. Oligo-Asp Tag/Zn(II) Complex Probe as a New Pair for Labelling and Fluorescence Imaging of Proteins. J. Am. Chem. Soc., 2006, vol. 128, no. 32, p. 10452-10459.

NP27 GAUTIER, Arnaud, et al. Selective Cross-Linking of Interacting Proteins Using Self-Labelling Tags. J. Am. Chem. Soc., 2009, vol. 131, no. 49, p. 17954-17962.

NP28 LEQUIN, Rudolf. Enzyme Immunoassay (EIA)/Enzyme-Linked Immunosorbent Assay (ELISA). Clinical Chemistry, 2005, vol. 51, 2415-2418.

NP29 ODELL, Ian, et al. Immunofluorescence techniques J. Invest. Dermatology, 2013, vol. 133, e4.

NP30 MAHMOOD, Tahrin, et al. Western Blot: Technique, Theory, and Trouble Shooting. North American Journal of Medical Sciences, 2012, vol. 4, 429-434.

NP31 HERMANSON, Greg. Bioconjugate Techniques. USA: Academic Press, 26 Jul 2010.

NP32 HUDSON, Philipp, et al. Engineered antibody fragments and the rise of single domains. Nat. Biotechnol., 2005, vol. 23, 1126-1136.

NP33 CABANTOUS, Stephanie, et al. Protein tagging and detection with engineered self-assembling fragments of green fluorescent protein. Nat. Biotechnol., 2005, vol. 23, 102-107.

NP34 LUKER, Kathryn, et al. Split Gaussia Luciferase for Imaging Ligand-Receptor Binding in Bioluminescent Imaging. Methods in Molecular Biology, 2013, vol. 1098, 59-69.

NP35 BUCHWALOW, Igor, et al. Immunochemistry: Basics and Methods. Springer: ISBN: 978-3-642-04608-7.

NP36 PALMER, J.L., et al. Reduction and re-oxidation of a critical disulphide bond in rabit antibidy molecule. J. Biol. Chem., 1963, vol. 238, no.7, p 2369-2398.

NP37 GE, Hui. UPA, a universal protein array system for quantitative detection of protein-protein, protein-DNA, protein-RNA and protein-ligand interactions. Nucleic Acids Res. 2000, vol. 28, e3.

NP38 ZHU, Heng, et al. Analysis of yeast protein kinases using protein chips. Nat. Genet., 2000, vol. 26, 283-289.

NP39 COHEN, Claudia, et al. A microchip-based enzyme assay for protein kinase A. Anal. Biochem., 1999, vol. 273, 89.

NP40 CHENG, Chao-Min, et al. Paper-Based ELISA. Angew. Chem., 2010, vol. 49, 4771-4774.

NP41 DULKEITH, E., et al. Fluorescence Quenching of Dye Molecules near Gold Nanoparticles: Radiative and Non-radiative Effects. Phys. Rev. Lett., 2002, vol. 89, 203002.

NP42 SPERLING, Ralph, et al. Biological applications of gold nanoparticles. Chem. Soc. Rev., 2008, vol. 37, 1896-19

NP43 DOLMANS, Dennis, et al. Photodynamic therapy for cancer. Nature reviews cancer, 2003, vol. 3, 380-387.

NP44 HAMBLIN, Michael, et al. Photodynamic therapy: a new antimicrobial approach to infectious disease? Photochem. Photobiol. Sci., 2004, vol. 3, 436-450.

### Aspects and Embodiments of the Invention

Aspects and embodiments of the invention may also be described in accordance with the following numbered paragraphs.

1. A multi-state dynamical hinge sensor comprising:
(a) a rod L and a rod R connected to each other by a joint C;
(b) the joint C comprises one or more elements connected end to end, where each element is selected to be rigid or flexible and is made of atoms or molecules;

(b) an atom or molecule or macromolecule B bonded to the end of L opposite to the joint;

(c) an atom or molecule or macromolecule B′ is bonded to the end of R opposite the joint;

(d) a state denoted as the ON state where B is attracted to B′ ;

(e) a state denoted as the OFF state where B is not attracted to B′;

(f) spacer atoms or molecules in various locations within the hinge;

(g) a number N constituent atoms or molecules q1, q2, . . . , qN of L and a number N′ constituent atoms or molecules q′1, q′2, . . . , q′N′ of R selected so that in the OFF state the hinge is open and in the ON state the hinge repeatedly opens and closes.

2. A multi-state dynamical hinge sensor as described in paragraph 1 to which is added: a fluorophore A or bioluminescent molecule and a fluorophore A′, wherein A is coupled directly to B or indirectly via the spacer atoms, and wherein A′ is coupled directly to B′ or indirectly via the spacer atoms, and wherein q1, q2, . . . , qN of L and q′1, q′2, . . . , q′N′ of R are adjustable so that in the OFF state the hinge is open and in the ON state the hinge repeatedly opens and closes.

3. The multi-state dynamical hinge sensor as described in paragraph 2 wherein A and A′ undergo measurable resonance energy transfer when sufficiently close to each other when B and B′ are in the ON state.

4. The multi-state dynamical hinge sensors described in paragraph 2 or 3 wherein one or both fluorophores or the bioluminescent molecule are photo-activatable or photo-convertible or photo-switchable or fluorescent protein timers or phosphorescent.

5. A multi-state dynamical hinge sensors described in paragraph 4 wherein the roles of (A and A′) and (B and B′) are exchanged.

6. The multi-state dynamical hinge sensors as described in paragraphs 4 to 5 wherein the constituents A,A′,B,B′,L,R, and spacers are comprised of either single amino acids, peptides or proteins and wherein q1, q2, . . . , qN, q′1, q′2, . . . , q′N′ are selected to be charged amino acids, or hydrophilic or hydrophobic amino acids, or a combination thereof.

7. The multi-state dynamical hinge sensors as described in paragraph 6 bonded directly or indirectly through A or A′ to any target protein or proteins of interest.

8. The multi-state dynamical hinge sensors as described in paragraph 7 wherein additional acceptor fluorophores absorb or emit light of different colours.

9. Copies of multi-state dynamical hinge sensors as described in paragraphs 7 or 8 and their insertions into sub-cellular locales described using plasmid and genetic-vector technology.

10. The use of multi-state dynamical hinge sensors described in paragraphs 7 to 9 to: (a) to visualise or monitor any of the following: (a) the structure and conformation of proteins; (b) the spatial distribution and assembly of protein complexes; (c) protein receptor/ligand interactions including the local concentrations of analytes; (d) the interactions of single molecules; (e) the structure and conformations of nucleic acids; (f) the distributions and transport of lipids; (g) membrane potential sensing; (h) monitoring fluorogenic protease substrates; (i) local cellular concentrations of cyclic AMP and calcium.

11. Use of the multi-state dynamical hinge sensors described in paragraphs 7 to 9 including within any of the following applications: biomolecular/molecular medicine and drug discovery; functional food and food for health research; development of ligands targeted at taste and olfactory receptors; cosmetics and perfume industries;

immunoassays and membrane fusion assays in biopharmaceuticals and pharmaceuticals industries.

12. Use of the multi-state dynamical hinge sensors described in paragraphs 7 to 9 for applications including medical diagnostics and forensic industries for automated DNA sequencing, real-time PCR assays, SNP detection, and detection of nucleic acid hybridization.

13. The use of the multi-state dynamical hinge sensors described in paragraphs 7 to 9 as light/ligand activated actuators or active agents in the targeted delivery of drugs;

and the manipulation and control of biological processes, and signalling networks.

14. The use of multi-state dynamical hinge sensors as described in paragraphs 2-5 in chemical, electronic, semi-conducting and quantum computing industries such as analytical indicators, electronic sensors, memory devices and nano-actuators.

15. Methods wherein multi-state dynamical hinge sensors described in paragraphs 7 to 9 are used including: biomolecular/molecular medicine and drug discovery; functional food and food for health research; development of ligands targeted at taste and olfactory receptors; cosmetics and perfume discovery; immunoassays and membrane fusion assays; medical diagnostics and forensic industries for automated DNA sequencing, real-time PCR assays, SNP detection, and detection of nucleic acid hybridization; targeted delivery of drugs; manipulation and control of biological processes, and signalling networks.

16. Methods wherein multi-state dynamical hinge sensors described in paragraphs 7 to 9 are used including: biomolecular/molecular medicine and drug discovery; functional food and food for health research; development of ligands targeted at taste and olfactory receptors; cosmetics and perfume discovery; immunoassays and membrane fusion assays; medical diagnostics and forensic industries for automated DNA sequencing, real-time PCR assays, SNP detection, and detection of nucleic acid hybridization; targeted delivery of drugs; manipulation and control of biological processes, and signalling networks.

17. Methods wherein multi-state dynamical hinge sensors described in paragraphs 7 to 9 are used to estimate the binding energies of ligands to receptors, and the ranking of their efficacy as agonists or antagonists in bimolecular/molecular medicine and drug discovery; functional food and food for health research; development of ligands targeted at taste and olfactory receptors; cosmetics and perfume discovery.

18. The multi-state dynamical hinge sensors as described in paragraph 6 and paragraph 7 where macromolecules A and A′ are selected to each be primary antibodies targeting different epitopes on the same analyte or antigen.

19. The multi-state dynamical hinge sensors as described in paragraph 6 and paragraph 7 where the macromolecule A is a primary antibody targeting an analyte or antigen, and and A′ is the corresponding secondary antibody.

20. The multi-state dynamical hinge sensors as described in paragraph 6 and paragraph 7 where the macromolecule A is an antigen targeting a primary antibody, and A′ is the corresponding secondary antibody.

21. The multi-state dynamical hinge sensors as described in paragraphs 6, 7, 17, 18, 19 where macromolecules B and B′ each comprise one or more selected molecules or moieties of split molecules, which, when brought close together due to the close presence of target analytes, undergo resonance energy transfer in the presence of a suitable electro-magnetic field of external or endogenous origin.

22. The multi-state dynamical hinge sensors as described in paragraphs 6, 7, 17, 18, 19 where macromolecules B and B′ each comprise one or more selected molecules or moieties of split molecules, which, when brought close together due to the close presence of target analytes, bioluminesce.

23. The multi-state dynamical hinge sensors as described in paragraphs 6, 7, 17, 18, 19 where macromolecules B and B′ each comprise one or more selected molecules or moieties of split molecules, which, when brought close together due to the close presence of target analytes, become an activated drug.

24. The multi-state dynamical hinge sensors as described in paragraphs 6, 7, 17, 18, 19 where macromolecules B and B′ each comprise one or more selected molecules or moieties of split molecules, which, when brought close together due to the close presence of target analytes, become an activated chemo-toxin.

25. The multi-state dynamical hinge sensors as described in paragraphs 6, 7, 17, 18, 19 where macromolecules B and B′ each comprise one or more selected molecules or moieties of split molecules, which, when brought close together due to the close presence of target analytes, become a chemical reaction or a catalysed chemical reaction.

26. The multi-state dynamical hinge sensors as described in paragraphs 6, 7, 17, 18, 19 where macromolecules B and B′ each comprise one or more selected molecules or moieties of split molecules in the presence of a suitable electro-magnetic field, which, when brought close together due to the presence of the target analytes, releases heat through quenching.

27. The multi-state dynamical hinge sensors as described in paragraphs 6, 7, 17, 18, 19 where macromolecules B and B′ each comprise one or more selected molecules or moieties of split molecules in the presence of a suitable electro-magnetic field, which, when brought close together due to the close presence of target analytes, become an activated photosensitizer complex producing oxygen radicals.

28. Multi-state dynamical hinge sensors with any combination of the effects described in paragraphs 20 to paragraph 26 in the same sensor.

29. The use of multi-state dynamical hinge sensors described in paragraphs 20, 21, 22 and 28 in the detection of analytes and their concentrations in assays and in living cells and their tracking over time.

30. The use of multi-state dynamical hinge sensors described in paragraphs 23 and 28 as a drug in the vicinity or within biological cells, fluids and tissue, and in vitro.

31. The use of multi-state dynamical hinge sensors described in paragraphs 24 and 28 as a chemotoxin in the vicinity or within biological cells, fluids and tissue, in vitro, and in organic materials.

32. The use of the multi-state dynamical hinge sensors described in paragraphs 25 and 28 as chemical reaction or a catalysed chemical reaction in the vicinity or within biological cells, fluids and tissue, in vitro, and in organic materials.

33. The use of the multi-state dynamical hinge sensors described in paragraphs 26 and 28 to deliver heat in the vicinity or within biological cells, fluids and tissue, in vitro, and in organic materials.

34. The use of the multi-state dynamical hinge sensors described in paragraphs 27 and 28 in photodynamic therapy in the vicinity or within biological cells, fluids and tissue, in vitro, and in organic materials.

35. Methods of use described in paragraph 28 wherein multi-state dynamical hinge sensors are used to perform assays for analytes including titration measure using microtiters or vials, with and without specialised equipment, for multiple applications including environmental, health, food safety, and security.

36. Methods of use described in paragraph 28 wherein multi-state dynamical hinge sensors is used to detect analytes in suitable continuous flow chambers for several applications including environmental, health, food safety, and security.

1. A sensor molecule for detecting a target molecule comprising:
(a) a rod-like molecule L and a rod-like molecule R connected to each other by a joint molecule C to form a hinge;
(b) a target binding molecule A bonded to the end of rod-like molecule L opposite to the joint molecule C;
(c) a binding molecule A′ bonded to the end of rod-like molecule R opposite the joint molecule C;
wherein the target binding molecule A is arranged to bind to the target molecule to be detected, and binding molecule A′ is arranged to bind to:
i) the same target molecule as target binding molecule A; or
ii) a complex of the target binding molecule A and the target; and
wherein the hinge is biased into an open position, such that target binding molecule A and binding molecule A′ are biased apart by the hinge.

2. The sensor molecule according to claim 1, wherein the presence and binding of a target molecule by target binding molecule A, and the binding of binding molecule A′ to either i) the target molecule or ii) the complex of the target binding molecule and target molecule, is arranged to bias the hinge into a closed position in opposition to the force of the hinge, which is biased to an open position.

3. The sensor molecule according to claim 1 or claim 2, wherein the sensor molecule comprises a state denoted as the ON state wherein A is attracted towards A′ and the hinge is arranged to repeatedly open and close; and a state denoted as the OFF state wherein the hinge is in an open position and A is not attracted towards A′.

4. The sensor molecule according to claim 3, wherein the ON state is detectable.

5. The sensor molecule according to any preceding claim, wherein the target binding molecule A and/or the binding molecule A′ are capable of emitting a signal for detection when they are in proximity to each other, or bound to each other.

6. The sensor molecule according to any preceding claim, wherein the sensor further comprises a signal molecule B and a signal molecule B′.

7. The sensor molecule according to claim 6, wherein the detectable ON state signal is provided by the pair of signal molecules B and B′ being brought into sufficient proximity to cause a detectable ON state signal to be emitted.

8. The sensor molecule according to claim 6 or claim 7, wherein the signal molecule B and/or B′ comprises a chromophore, fluorophore or bioluminescent molecule; and/or the target binding molecule A and/or binding molecule A′ comprises a chromophore, fluorophore or bioluminescent molecule.

9. The sensor molecule according to any of claims 4 to 8, wherein the detectable ON state signal is provided by resonance energy transfer (RET) between target binding molecule A and binding molecule A′.

10. The sensor molecule according to any of claims 6 to 9, wherein the detectable ON state signal is provided by resonance energy transfer (RET) between signal molecule B and signal molecule B′.

11. The sensor molecule according to claim 9 or 10, wherein the resonance energy transfer (RET) is Förster resonance energy transfer (FRET) or bioluminescent resonance energy transfer (BRET).

12. The sensor molecule according to any of claims 6 to 11, wherein the signal molecule B is bound to ligand binding molecule A and the signal molecule B′ is bound to binding molecule A′ or vice versa.

13. The sensor molecule according to any of claims 6 to 12, where B and B′ are respectively bound to A and A′, or vice versa, the binding is via a spacer molecule.

14. The sensor molecule according to any of claims 6 to 13, wherein the sensor molecules B and B′ each comprise a part of a split molecule.

15. The sensor molecule according to claim 14, wherein the split molecule comprises a split fluorescent protein.

16. The sensor molecule according to claim 14, wherein the split molecule comprises a biological active molecule that can be split into two or more parts, such that when the parts are brought back together in the presence of a target molecule of the sensor molecule, the function of the biological active molecule is restored.

17. The sensor molecule according to claim 16, wherein the biological active molecule comprises an active drug, a pro-drug, an enzyme, or a co-factor.

18. The sensor molecule according to any of claims 14 to 17, wherein the split molecule comprises a toxin.

19. The sensor molecule according to claim 18, wherein the toxin is the A and B components of an AB protein toxin.

20. The sensor molecule according to any preceding claim, wherein the aspect ratio of the rod-like molecules L and R is about 6-10:1 (length to width).

21. The sensor molecule according to any preceding claim, wherein the rod-like molecules L and R are each at least 40 Ångströms in length.

22. The sensor molecule according to any preceding claim, wherein the rod-like molecules L and R are substantially rigid.

23. The sensor molecule according to any preceding claim, wherein the rod-like molecule L and/or rod-like molecule R comprise or consist of polypeptide.

24. The sensor molecule according to any preceding claim, wherein the rod-like molecule L and/or rod-like molecule R comprise or consist of an alpha-helical polypeptide.

25. The sensor molecule according to any preceding claim, wherein the rod-like molecule L comprises a number N of constituent molecules q1, q2, . . . , qN; and the rod-like molecule R comprises a number N′ of constituent molecules q′1, q′2, . . . , q′N′;
wherein q1, q2, . . . , qN, q′1, q′2, . . . , q′N′ are selected to be charged amino acids, or hydrophilic or hydrophobic amino acids, or a combination thereof.

26. The sensor molecule according to any preceding claim, wherein the rod-like molecules L and R of the sensor molecule are symmetrical in sequence.

27. The sensor molecule according to any preceding claim, wherein the binding energy of A and A′ are substantially similar to the opposing bias energy of the hinge.

28. The sensor molecule according to any preceding claim, wherein the rod-like molecules L and R comprise an alpha helix of the following repeat residues [EAAAK(SEQ ID NO: 106)]^{m} and [KAAAE(SEQ ID NO: 107)]^{m} respectively, where m is the number of repeats ranging from 6 to 12, and E and K are positively charged at physiological pH condition; or
wherein the rod-like molecules L and R comprise an alpha helix of the following repeat residues and [EAAAAK (SEQ ID NO: 108)]^{m} and [KAAAAE(SEQ ID NO: 109)]^{m} respectively, where m is the number of repeats ranging from 6 to 12, and E and K are positively charged at physiological pH condition; or
wherein the the rod-like molecules L and R comprise an alpha helix of the following repeat residues [EAAAAAK(SEQ ID NO: 110)]^{m} and [KAAAAAE(SEQ ID NO: 111)]^{m} respectively, where m is the number of repeats ranging from 6 to 12, and E and K are positively charged at physiological pH condition.

29. The sensor molecule according to any preceding claim, wherein the joint molecule C is flanked according to the following sequence [EAAAAAK(SEQ ID NO: 110)]⁴ EAAKAAKA(SEQ ID NO: 112)-[Joint Molecule C]-AKAAKAAE(SEQ ID NO: 113) [KAAAAAE(SEQ ID NO: 111)]⁴.

30. The sensor molecule according to any preceding claim, wherein the rod-like molecule L and R together with the joint molecule C comprise the sequence [EAAAAAK(SEQ ID NO: 110)]⁴ EAAKAAKA(SEQ ID NO: 112) S G S AKAAKAAE(SEQ ID NO: 113) [KAAAAAE(SEQ ID NO: 111)]⁴.

31. The sensor molecule according to any preceding claim, wherein the sensor molecule is a fusion protein.

32. The sensor molecule according to any preceding claim, wherein the sensor molecule comprises or consists of the protein sequence FP1-A[GSG]^{m1} A (SEQ ID NO: 114)-TBM-A[GSG]^{m2} A L (SEQ ID NO: 114)-[hinge]-R A[GSG]^{m3} A (SEQ ID NO: 114)-BM-A[GSG]^{m4} A (SEQ ID NO: 114)-FP2,
wherein FP1 and FP2 are a signal molecule B and B′ respectively;
TBM and BM are the target binding molecule A and binding molecule A′ respectively;
L and R denote the Left and Right alpha helices of the hinge;
A, S, and G denote the amino acids Alanine, Glycine and Serine; and
m1, m2, m3 and m4 are appropriately selected number of repeats to ensure that the sensor is functional according to the invention.

33. The sensor molecule according to any preceding claim, wherein the rod-like molecules L and R and joint molecule C (the hinge) are composed of residue sequences such as:
[EAAAK(SEQ ID NO: 106)]ⁿ A[joint molecule C]^{m} A [KAAAE(SEQ ID NO: 107)]ⁿ; or
[EAAAK(SEQ ID NO: 106)]ⁿ A[joint molecule C]^{m} A [KAAAE(SEQ ID NO: 107)]ⁿ,
wherein E, A, G, S, and K are the single letter codes for amino acids and n and m are non-zero positive integers.

34. The sensor molecule according to any preceding claim, wherein the joint molecule C is flexible.

35. The sensor molecule according to any preceding claim, wherein the joint molecule C comprises or consists of amino acids.

36. The sensor molecule according to any preceding claim, wherein the joint molecule C comprises or consists of the amino acid glycine.

37. The sensor molecule according to any preceding claim, wherein the joint molecule C comprises the amino acid sequence SGS or GS.

38. The sensor molecule according to any preceding claim, wherein the target binding molecule A and/or binding molecule A′ comprises any one of an antibody, antibody fragment or mimic thereof; an antigen, for example a protein or peptide, which is capable of being bound by an antibody; a receptor protein, which comprises a ligand binding site; a ligand, which is capable of being bound by a receptor molecule; or nucleic acid.

39. The sensor molecule according to any preceding claim, wherein the sensor molecule is bound to another sensor molecule according to any preceding claim.

40. A nucleic acid encoding the sensor molecule according to any preceding claim.

41. The nucleic acid according to claim 40, wherein the entire sensor molecule is encoded as a fusion protein.

42. The nucleic acid according to claim 40 or 41, wherein the nucleic acid comprises or consists of a vector.

43. A host cell comprising the nucleic acid according to any of claims 40 to 42; and/or comprising the sensor molecule according to any of claims 1 to 39.

44. A composition comprising the sensor molecule according to any of claims 1 to 39, or the nucleic acid according to any of claims 40 to 42; optionally, wherein the composition is a pharmaceutically acceptable composition.

45. An assay method for the detection of a target molecule in sample comprising:
providing the sample;
providing the sensor molecule according to any of claims 1 to 39 in the sample;
detecting the presence or absence of a signal from the sensor molecule;
wherein an ON signal confirms the presence of the target molecule in the sample.

46. An assay method for the detection of a target molecule in vivo comprising:
providing the sensor molecule according to any of claims 1 to 39 in vivo;
detecting the presence or absence of a signal from the sensor molecule;
wherein an ON signal confirms the presence of the target molecule in vivo.

47. Use of the sensor molecule according to any of claims 1 to 39 to visualise or monitor any of the following: (a) the structure and conformation of proteins; (b) the spatial distribution and assembly of protein complexes; (c) protein receptor/ligand interactions including the local concentrations of analytes; (d) the interactions of single molecules; (e) the structure and conformations of nucleic acids; (f) the distributions and transport of lipids; (g) membrane potential sensing; (h) monitoring fluorogenic protease substrates; (i) local cellular concentrations of cyclic AMP and calcium.

48. Use of the sensor molecule according to any of claims 1 to 39 in the detection of a target analyte, and optionally its concentration, in assays or living cells; or
as a drug or drug delivery vehicle to, or within, biological cells, fluids or tissue; or
to provide or catalyse a chemical reaction in the vicinity or within biological cells, organic materials, fluids or tissue; or
to deliver heat in the vicinity or within biological cells, fluids, tissue or organic materials; or
in photodynamic therapy in the vicinity or within biological cells, fluids, tissue, or organic materials; or
to perform assays for analytes including titration measure using microtiters or vials, with and without specialised equipment; or to detect analytes in suitable continuous flow chambers.

49. Use of the sensor molecule according to any of claims 1 to 39 for cell killing, wherein the sensor molecule comprises a split molecule that is an active toxin once the parts of the split molecule are brought together in the presence of a target molecule.

50. A method of providing a biological active only in the presence of a target molecule comprising:
providing the sensor molecule according to any of claims 1 to 39, wherein the sensor molecule comprises a split molecule, wherein the split molecule is a biological active.

51. A method of treatment for a disease in a subject comprising the administration of the sensor molecule according to any of claims 1 to 39 or the composition according to claim 43, wherein the sensor molecule comprises a biological active in the form of a split molecule, which is capable of becoming an active suitable for treatment of the disease.

52. The sensor molecule according to any of claims 1 to 39 or the composition according to claim 44, for use as medicament; optionally for use to treat cancer in a subject.

53. The sensor molecule, use, method or composition substantially as described herein; optionally with reference to the accompanying drawings (except for FIG. 1).


SEQUENCE LISTING (128 sequences)

SEQ ID NO: 1 - protein, 749 aa, Homo sapiens
GENITQPTQQSTQATQRFLIEKFSQEQIGENIVCRVICTTGQIPIRDLSADISQVLKEKRSIKKVWTFGRNPACDYHLGNISRLSNKHFQILLGEDGNLLLNDISTNGTWLNGQKVEKNSNQLLSQGDEITVGVGVESDILSLVIFINDKFKQCLEQNKVDRIRAGSGGSGGSGAMSKGEELFTGVVPILVELDGDVNGHKFSVSGEGEGDATYGKLTLKFICTTGKLPVPWPTLVTTLVQCFSRYPDHMKRHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGIAGSGGSGGSGAEAAAKEAAAKEAAAKEAAAKEAAAKEAAAKAGSGAKAAAEKAAAEKAAAEKAAAEKAAAEKAAAEAGSGGSGGSGAMSKGEELFTGVVPILVELDGDVNGHKFSVSGEGEGDATYGKLTLKFICTTGKLPVPWPTLVTTFLQCFARYPDHMKRHDFFKSAMPEGYVQERTIFFKDDGNYKTRAEVKFEGDTLVNRIELKGIDFKEDGNILGHKLEYNYNSQNVYIMADKQKNGIKVNFKIRHNIEDGSVQLADHYQQNTPIGDGPVLLPDNHYLSYQSALSKDPNEKRDHMVLLEFVTAAGIAGSGASAGKPGSGEGSTKGLRRATVLDGGTGGSEL

SEQ ID NO: 2 - protein, 533 aa, Homo sapiens
  misc_feature (241)..(241): Xaa can be any naturally occurring amino acid
GENITQPTQQSTQATQRFLIEKFSQEQIGENIVCRVICTTGQIPIRDLSADISQVLKEKRSIKKVWTFGRNPACDYHLGNISRLSNKHFQILLGEDGNLLLNDISTNGTWLNGQKVEKNSNQLLSQGDEITVGVGVESDILSLVIFINDKFKQCLEQNKVDRIRAGSGGSGGSGAMSKGEELFTGVVPILVELDGDVNGHKFSVSGEGEGDATYGKLTLKFICTTGKLPVPWPTLVTTFGXGLQCFARYPDHMKRHDFFKSAMPEGYVQERTIFFKDDGNYKTRAEVKFEGDTLVNRIELKGIDFKEDGNILGHKAGSGGSGGSGAEAAAKEAAAKEAAAKEAAAKEAAAKEAAAKSGSKAAAEKAAAEKAAAEKAAAEKAAAEKAAAEAGSGGSGGSGALEYNYNSQNVYIMADKQKNGIKVNFKIRHNIEDGSVQLADHYQQNTPIGDGPVLLPDNHYLSYQSALSKDPNEKRDHMVLLEFVTAAGITHGMDELYKAGSGASAGKPGSGEGSTKGLRRATVLDGGTGGSEL

SEQ ID NO: 3 - protein, 66 aa, Homo sapiens
EAAAKEAAAKEAAAKEAAAKEAAAKEAAAKAGSGSAKAAAEKAAAEKAAAEKAAAEKAAAEKAAAE

SEQ ID NO: 4 - protein, 6 aa, Homo sapiens
GSGGSG

SEQ ID NO: 5 - protein, 9 aa, Homo sapiens
GSGGSGGSG

SEQ ID NO: 6 - protein, 12 aa, Homo sapiens
GSGGSGGSGGSG

SEQ ID NO: 7 - protein, 15 aa, Homo sapiens
GSGGSGGSGGSGGSG

SEQ ID NO: 8 - protein, 5 aa, Homo sapiens
AGSGA

SEQ ID NO: 9 - protein, 8 aa, Homo sapiens
AGSGGSGA

SEQ ID NO: 10 - protein, 11 aa, Homo sapiens
AGSGGSGGSGA

SEQ ID NO: 11 - protein, 14 aa, Homo sapiens
AGSGGSGGSGGSGA

SEQ ID NO: 12 - protein, 17 aa, Homo sapiens
AGSGGSGGSGGSGGSGA

SEQ ID NO: 13 - protein, 30 aa, Homo sapiens
EAAAKEAAAKEAAAKEAAAKEAAAKEAAAK

SEQ ID NO: 14 - protein, 35 aa, Homo sapiens
EAAAKEAAAKEAAAKEAAAKEAAAKEAAAKEAAAK

SEQ ID NO: 15 - protein, 40 aa, Homo sapiens
EAAAKEAAAKEAAAKEAAAKEAAAKEAAAKEAAAKEAAAK

SEQ ID NO: 16 - protein, 45 aa, Homo sapiens
EAAAKEAAAKEAAAKEAAAKEAAAKEAAAKEAAAKEAAAKEAAAK

SEQ ID NO: 17 - protein, 50 aa, Homo sapiens
EAAAKEAAAKEAAAKEAAAKEAAAKEAAAKEAAAKEAAAKEAAAKEAAAK

SEQ ID NO: 18 - protein, 55 aa, Homo sapiens
EAAAKEAAAKEAAAKEAAAKEAAAKEAAAKEAAAKEAAAKEAAAKEAAAKEAAAK

SEQ ID NO: 19 - protein, 60 aa, Homo sapiens
EAAAKEAAAKEAAAKEAAAKEAAAKEAAAKEAAAKEAAAKEAAAKEAAAKEAAAKEAAAK

SEQ ID NO: 20 - protein, 30 aa, Homo sapiens
KAAAEKAAAEKAAAEKAAAEKAAAEKAAAE

SEQ ID NO: 21 - protein, 35 aa, Homo sapiens
KAAAEKAAAEKAAAEKAAAEKAAAEKAAAEKAAAE

SEQ ID NO: 22 - protein, 40 aa, Homo sapiens
KAAAEKAAAEKAAAEKAAAEKAAAEKAAAEKAAAEKAAAE

SEQ ID NO: 23 - protein, 45 aa, Homo sapiens
KAAAEKAAAEKAAAEKAAAEKAAAEKAAAEKAAAEKAAAEKAAAE

SEQ ID NO: 24 - protein, 50 aa, Homo sapiens
KAAAEKAAAEKAAAEKAAAEKAAAEKAAAEKAAAEKAAAEKAAAEKAAAE

SEQ ID NO: 25 - protein, 55 aa, Homo sapiens
KAAAEKAAAEKAAAEKAAAEKAAAEKAAAEKAAAEKAAAEKAAAEKAAAEKAAAE

SEQ ID NO: 26 - protein, 60 aa, Homo sapiens
KAAAEKAAAEKAAAEKAAAEKAAAEKAAAEKAAAEKAAAEKAAAEKAAAEKAAAEKAAAE

SEQ ID NO: 27 - protein, 36 aa, Homo sapiens
EAAAAKEAAAAKEAAAAKEAAAAKEAAAAKEAAAAK

SEQ ID NO: 28 - protein, 42 aa, Homo sapiens
EAAAAKEAAAAKEAAAAKEAAAAKEAAAAKEAAAAKEAAAAK

SEQ ID NO: 29 - protein, 48 aa, Homo sapiens
EAAAAKEAAAAKEAAAAKEAAAAKEAAAAKEAAAAKEAAAAKEAAAAK

SEQ ID NO: 30 - protein, 54 aa, Homo sapiens
EAAAAKEAAAAKEAAAAKEAAAAKEAAAAKEAAAAKEAAAAKEAAAAKEAAAAK

SEQ ID NO: 31 - protein, 60 aa, Homo sapiens
EAAAAKEAAAAKEAAAAKEAAAAKEAAAAKEAAAAKEAAAAKEAAAAKEAAAAKEAAAAK

SEQ ID NO: 32 - protein, 66 aa, Homo sapiens
EAAAAKEAAAAKEAAAAKEAAAAKEAAAAKEAAAAKEAAAAKEAAAAKEAAAAKEAAAAKEAAAAK

SEQ ID NO: 33 - protein, 72 aa, Homo sapiens
EAAAAKEAAAAKEAAAAKEAAAAKEAAAAKEAAAAKEAAAAKEAAAAKEAAAAKEAAAAKEAAAAKEAAAAK

SEQ ID NO: 34 - protein, 36 aa, Homo sapiens
KAAAAEKAAAAEKAAAAEKAAAAEKAAAAEKAAAAE

SEQ ID NO: 35 - protein, 42 aa, Homo sapiens
KAAAAEKAAAAEKAAAAEKAAAAEKAAAAEKAAAAEKAAAAE

SEQ ID NO: 36 - protein, 48 aa, Homo sapiens
KAAAAEKAAAAEKAAAAEKAAAAEKAAAAEKAAAAEKAAAAEKAAAAE

SEQ ID NO: 37 - protein, 54 aa, Homo sapiens
KAAAAEKAAAAEKAAAAEKAAAAEKAAAAEKAAAAEKAAAAEKAAAAEKAAAAE

SEQ ID NO: 38 - protein, 60 aa, Homo sapiens
KAAAAEKAAAAEKAAAAEKAAAAEKAAAAEKAAAAEKAAAAEKAAAAEKAAAAEKAAAAE

SEQ ID NO: 39 - protein, 66 aa, Homo sapiens
KAAAAEKAAAAEKAAAAEKAAAAEKAAAAEKAAAAEKAAAAEKAAAAEKAAAAEKAAAAEKAAAAE

SEQ ID NO: 40 - protein, 72 aa, Homo sapiens
KAAAAEKAAAAEKAAAAEKAAAAEKAAAAEKAAAAEKAAAAEKAAAAEKAAAAEKAAAAEKAAAAEKAAAAE

SEQ ID NO: 41 - protein, 42 aa, Homo sapiens
EAAAAAKEAAAAAKEAAAAAKEAAAAAKEAAAAAKEAAAAAK

SEQ ID NO: 42 - protein, 49 aa, Homo sapiens
EAAAAAKEAAAAAKEAAAAAKEAAAAAKEAAAAAKEAAAAAKEAAAAAK

SEQ ID NO: 43 - protein, 56 aa, Homo sapiens
EAAAAAKEAAAAAKEAAAAAKEAAAAAKEAAAAAKEAAAAAKEAAAAAKEAAAAAK

SEQ ID NO: 44 - protein, 63 aa, Homo sapiens
EAAAAAKEAAAAAKEAAAAAKEAAAAAKEAAAAAKEAAAAAKEAAAAAKEAAAAAKEAAAAAK

SEQ ID NO: 45 - protein, 70 aa, Homo sapiens
EAAAAAKEAAAAAKEAAAAAKEAAAAAKEAAAAAKEAAAAAKEAAAAAKEAAAAAKEAAAAAKEAAAAAK

SEQ ID NO: 46 - protein, 77 aa, Homo sapiens
EAAAAAKEAAAAAKEAAAAAKEAAAAAKEAAAAAKEAAAAAKEAAAAAKEAAAAAKEAAAAAKEAAAAAKEAAAAAK

SEQ ID NO: 47 - protein, 84 aa, Homo sapiens
EAAAAAKEAAAAAKEAAAAAKEAAAAAKEAAAAAKEAAAAAKEAAAAAKEAAAAAKEAAAAAKEAAAAAKEAAAAAKEAAAAAK

SEQ ID NO: 48 - protein, 42 aa, Homo sapiens
KAAAAAEKAAAAAEKAAAAAEKAAAAAEKAAAAAEKAAAAAE

SEQ ID NO: 49 - protein, 49 aa, Homo sapiens
KAAAAAEKAAAAAEKAAAAAEKAAAAAEKAAAAAEKAAAAAEKAAAAAE

SEQ ID NO: 50 - protein, 56 aa, Homo sapiens
KAAAAAEKAAAAAEKAAAAAEKAAAAAEKAAAAAEKAAAAAEKAAAAAEKAAAAAE

SEQ ID NO: 51 - protein, 63 aa, Homo sapiens
KAAAAAEKAAAAAEKAAAAAEKAAAAAEKAAAAAEKAAAAAEKAAAAAEKAAAAAEKAAAAAE

SEQ ID NO: 52 - protein, 70 aa, Homo sapiens
KAAAAAEKAAAAAEKAAAAAEKAAAAAEKAAAAAEKAAAAAEKAAAAAEKAAAAAEKAAAAAEKAAAAAE

SEQ ID NO: 53 - protein, 77 aa, Homo sapiens
KAAAAAEKAAAAAEKAAAAAEKAAAAAEKAAAAAEKAAAAAEKAAAAAEKAAAAAEKAAAAAEKAAAAAEKAAAAAE

SEQ ID NO: 54 - protein, 84 aa, Homo sapiens
KAAAAAEKAAAAAEKAAAAAEKAAAAAEKAAAAAEKAAAAAEKAAAAAEKAAAAAEKAAAAAEKAAAAAEKAAAAAEKAAAAAE

SEQ ID NO: 55 - protein, 8 aa, Homo sapiens
EAAKAAKA

SEQ ID NO: 56 - protein, 8 aa, Homo sapiens
AKAAKAAE

SEQ ID NO: 57 - protein, 5 aa, Homo sapiens
AGSGA

SEQ ID NO: 58 - protein, 8 aa, Homo sapiens
AGSGGSGA

SEQ ID NO: 59 - protein, 11 aa, Homo sapiens
AGSGGSGGSGA

SEQ ID NO: 60 - protein, 14 aa, Homo sapiens
AGSGGSGGSGGSGA

SEQ ID NO: 61 - protein, 17 aa, Homo sapiens
AGSGGSGGSGGSGGSGA

SEQ ID NO: 62 - protein, 20 aa, Homo sapiens
AGSGGSGGSGGSGGSGGSGA

SEQ ID NO: 63 - protein, 23 aa, Homo sapiens
AGSGGSGGSGGSGGSGGSGGSGA

SEQ ID NO: 64 - protein, 26 aa, Homo sapiens
AGSGGSGGSGGSGGSGGSGGSGGSGA

SEQ ID NO: 65 - protein, 29 aa, Homo sapiens
AGSGGSGGSGGSGGSGGSGGSGGSGGSGA

SEQ ID NO: 66 - protein, 32 aa, Homo sapiens
AGSGGSGGSGGSGGSGGSGGSGGSGGSGGSGA

SEQ ID NO: 67 - protein, 63 aa, Homo sapiens
EAAAKEAAAKEAAAKEAAAKEAAAKEAAAKSGSKAAAEKAAAEKAAAEKAAAEKAAAEKAAAE

SEQ ID NO: 68 - protein, 61 aa, Homo sapiens
EAAAKEAAAKEAAAKEAAAKEAAAKEAAAKEAAAKEAAAKEAAAKEAAAKEAAAKEAAAKA

SEQ ID NO: 69 - protein, 66 aa, Homo sapiens
EAAAKEAAAKEAAAKEAAAKEAAAKEAAAKEAAAKEAAAKEAAAKEAAAKEAAAKEAAAKEAAAKA

SEQ ID NO: 70 - protein, 71 aa, Homo sapiens
EAAAKEAAAKEAAAKEAAAKEAAAKEAAAKEAAAKEAAAKEAAAKEAAAKEAAAKEAAAKEAAAKEAAAKA

SEQ ID NO: 71 - protein, 76 aa, Homo sapiens
EAAAKEAAAKEAAAKEAAAKEAAAKEAAAKEAAAKEAAAKEAAAKEAAAKEAAAKEAAAKEAAAKEAAAKEAAAKA

SEQ ID NO: 72 - protein, 81 aa, Homo sapiens
EAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 73 - protein, 86 aa, Homo sapiens
EAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 74 - protein, 91 aa, Homo sapiens
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 75 - protein, 96 aa, Homo sapiens
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 76 - protein, 101 aa, Homo sapiens
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 77 - protein, 106 aa, Homo sapiens
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 78 - protein, 111 aa, Homo sapiens
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 79 - protein, 116 aa, Homo sapiens
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 80 - protein, 121 aa, Homo sapiens
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 81 - protein, 36 aa, Homo sapiens
EAAAAAKEAAAAAKEAAAAAKEAAAAAKEAAKAAKA

SEQ ID NO: 82 - protein, 75 aa, Homo sapiens
EAAAAAKEAAAAAKEAAAAAKEAAAAAKEAAKAAKASGSAKAAKAAEKAAAAAEKAAAAAEKAAAAAEKAAAAAE

SEQ ID NO: 83 - protein, 6 aa, Homo sapiens
SGSSGS

SEQ ID NO: 84 - protein, 9 aa, Homo sapiens
SGSSGSSGS

SEQ ID NO: 85 - protein, 12 aa, Homo sapiens
SGSSGSSGSSGS

SEQ ID NO: 86 - protein, 15 aa, Homo sapiens
SGSSGSSGSSGSSGS

SEQ ID NO: 87 - protein, 18 aa, Homo sapiens
SGSSGSSGSSGSSGSSGS

SEQ ID NO: 88 - protein, 21 aa, Homo sapiens
SGSSGSSGSSGSSGSSGSSGS

SEQ ID NO: 89 - protein, 24 aa, Homo sapiens
SGSSGSSGSSGSSGSSGSSGSSGS

SEQ ID NO: 90 - protein, 27 aa, Homo sapiens
SGSSGSSGSSGSSGSSGSSGSSGSSGS

SEQ ID NO: 91 - protein, 30 aa, Homo sapiens
SGSSGSSGSSGSSGSSGSSGSSGSSGSSGS

SEQ ID NO: 92 - protein, 4 aa, Homo sapiens
SGSG

SEQ ID NO: 93 - protein, 6 aa, Homo sapiens
SGSGSG

SEQ ID NO: 94 - protein, 8 aa, Homo sapiens
SGSGSGSG

SEQ ID NO: 95 - protein, 10 aa, Homo sapiens
SGSGSGSGSG

SEQ ID NO: 96 - protein, 12 aa, Homo sapiens
SGSGSGSGSGSG

SEQ ID NO: 97 - protein, 14 aa, Homo sapiens
SGSGSGSGSGSGSG

SEQ ID NO: 98 - protein, 16 aa, Homo sapiens
SGSGSGSGSGSGSGSG

SEQ ID NO: 99 - protein, 18 aa, Homo sapiens
SGSGSGSGSGSGSGSGSG

SEQ ID NO: 100 - protein, 20 aa, Homo sapiens
SGSGSGSGSGSGSGSGSGSG

SEQ ID NO: 101 - protein, 799 aa, Homo sapiens
GENTTQPTQQSTQATQRFLIEKFSQEQIGENIVCRVICTTGQIPIRDLSADISQVLICEICRSIKKVWTFGRNPACDYHLGNISRLSNICHFQILLGEDGNLLLNDISTNGTWLNGQKVEICNSNQLLSQGDEITVGVGVESDILSLVIFINDKFKQCLEQNKVDRIRAGSGGSGGSGAMSKGEELFTGVVPILVELDGDVNGHICFSVSGEGEGDATYGICLTLKFICTIGICLPVPWPTLVTTLTWGVQCFSRYPDHMICRHDFFKSAMPEGYVQERTIFFICDDGNYICTRAEVKFEGDTLVNRIELKGIDFICEDGNILGHICLEYNYISHNVYITADKQICNGIICANFICIRBNIEDGSVQLADHYQQNTPIGDGPVLLPDNHYLSTQSALSICDPNEICRDHMVLLEFVTAAGIAGSGGSGGSGAEAAAICEAAAICEAAAICEAAAICEAAAICEAAAICAGSGAICAAAEICAAAEICAAAEICAAAEICAAAEKAAAEAGSGGSGGSGAMSKGEELFTGVVPILVELDGDVNGHICFSVSGEGEGDATYGICLTLKFICTTGICLPVPWPTLVTTLTWGVQCFSRYPDHMKRHDFFKSAMPEGYVQERTIFFKDDGNYKFRAEVKFEGDTLVNRIELKGIDFICEDGNILGHICLEYNYISHNVYITADKQICNGIICANFICIRBNIEDGSVQLADHYQQNTPIGDGPVLLPDNHYLSTQSALSICDPNEICRDHMVLLEFVTAAGIAGSGGSGGSGASAGKPGSGEGSTKGLRRATVLDGGTGGSEL

SEQ ID NO: 102 - protein, 797 aa, Homo sapiens
  misc_feature (245)..(245): Xaa can be any naturally occurring amino acid
GENITQPTQQSTQATQRFLIEKESQEQIGENIVCRVICTTGQIPIRDLSADISQVLKEICRSIKKVWTFGRNPACDYHLGNISRLSNICHFQILLGEDGNLLLNDISTNGTWLNGQKVEICNSNQLLSQGDEITVGVGVESDILSLVIFINDKFKQCLEQNKVDRIRAGSGGSGGSGAMSKGEELFTGVVPILVELDGDVNGHKFSVSGEGEGDATYGKLTLKFICTTGKLPVPWPTLVATTEGXGLQCFARYPDHMICRHDFFKSAMPEGYVQERTIFFICDDGNYKTRAEVICFEGDTLVNRIELKGIDFKEDGNILGHKAGSGGSGGSGAEAAAKEAAAKEAAAKEAAAKEAAAKEAAAKSGSKAAAEICAAAEICAAAEICAAAEICAAAEKAAAEAGSGGSGGSGALEYNYNSQNVYIMADKQICNGIKVNFKIRHNIEDGSVQLADHYQQNTPIGDGPVLLPDNHYLSYQSALSICDPNEICRDHMVLLEFVTAAGITHGMDELYKAGSGGSGGSGAMSKGEELFTGVVPILVELDGDVNGHKFSVSGEGEGDATYGKLTLKFICTTGKLPVPWPTLVTTLTWGVQCFSRYPDHMICRHDFFKSAMPEGYVQERTIFFICDDGNYKTRAEVICFEGDTLVNRIELKGIDEKEDGNILGHKLEYNYISHNVYITADKQICNGIKANFKIRHNIEDGSVQLADHYQQNTPIGDGPVLLPDNHYLSTQSALSKDPNEKRDHMVLLEFVTAAGIAGSGGSGGSGASAGKPGSGEGSTKGLRRATVLDGGTGGSEL

SEQ ID NO: 103 - protein, 833 aa, Homo sapiens
  misc_feature (201)..(201): Xaa can be any naturally occurring amino acid
QVQLQESGGGLVQPGGSLRLSCAASGSISSITTMGWYRQDPGKGRELVALINSVGDTTYAGSGRFTISRDNAKNTVYLEMSSLKPEDTAVYYCNAFMSTNSGRTGSFWGQGTQVTVSSHHHHHHAGSGGSGGSGAMSKGEELFTGVVPILVELDGDVNGHKFSVSGEGEGDATYGKLTLKFICTTGKLPVPWPTLVTTFGXGLQCFARYPDHMKRHDFFKSAMPEGYVQERTIFFKDDGNYKTRAEVKFEGDTLVNRIELKGIDFKEDGNILGHKAGSGGSGGSGAEAAAKEAAAKEAAAKEAAAKEAAAKEAAAKSGSKAAAEKAAAEKAAAEKAAAEKAAAEKAAAEAGSGGSGGSGALEYNYNSQNVYIMADKQKNGIKVNFKIRHNIEDGSVQLADHYQQNTPIGDGPVLLPDNHYLSYQSALSKDPNEKRDHMVLLEFVTAAGITHGMDELYKAGSGGSGGSGAMSKGEELFTGVVPILVELDGDVNGHKFSVSGEGEGDATYGKLTLKFICTTGKLPVPWPTLVTTLTWGVQCFSRYPDHMKRHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGIAGSGGSGGSGAQVQLQESGGGLVQPGGSLRLSCAASGSISSITTMGWYRQDPGKGRELVALINSVGDTTYAGSGRFTISRDNAKNTVYLEMSSLKPEDTAVYYCNAFMSTNSGRTGSFWGQGTQVTVSSHHHHHH

SEQ ID NO: 104 - protein, 1065 aa, Homo sapiens
  misc_feature (317)..(317): Xaa can be any naturally occurring amino acid
DYKDIVLTQSHKFMSTSVGDRVSITCKASQDVGTAVAWYQQKPGQSPKLLIYWASTRHTGVPDRFTGSGSGTDFTLTISNVQSEDLADYFCQQYSSYPLTFGAGTKLELKRGGGGSGGGGSGGGGSGGGGSQVQLQEPGGELVRPGASVKLSCKASGYTFTSYWINWVKQRPGQGLEWIGNIYPSDSYTNYNQKFKDKATLTVDKSSSTAYMQLSSLTSEDSAVYFCARWGYWGQGTLVTAGSGGSGGSGAMSKGEELFTGVVPILVELDGDVNGHKFSVSGEGEGDATYGKLTLKFICTTGKLPVPWPTLVTTFGXGLQCFARYPDHMKRHDFFKSAMPEGYVQERTIFFKDDGNYKTRAEVKFEGDTLVNRIELKGIDFKEDGNILGHKAGSGGSGGSGAEAAAKEAAAKEAAAKEAAAKEAAAKEAAAKSGSKAAAEKAAAEKAAAEKAAAEKAAAEKAAAEAGSGGSGGSGALEYNYNSQNVYIMADKQKNGIKVNFKIRHNIEDGSVQLADHYQQNTPIGDGPVLLPDNHYLSYQSALSKDPNEKRDHMVLLEFVTAAGITHGMDELYKAGSGGSGGSGAMSKGEELFTGVVPILVELDGDVNGHKFSVSGEGEGDATYGKLTLKFICTTGKLPVPWPTLVTTLTWGVQCFSRYPDHMKRHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGIAGSGGSGGSGADYKDIVLTQSHKFMSTSVGDRVSITCKASQDVGTAVAWYQQKPGQSPKLLIYWASTRHTGVPDRFTGSGSGTDFTLTISNVQSEDLADYFCQQYSSYPLTFGAGTKLELKRGGGGSGGGGSGGGGSGGGGSQVQLQEPGGELVRPGASVKLSCKASGYTFTSYWINWVKQRPGQGLEWIGNIYPSDSYTNYNQKFKDKATLTVDKSSSTAYMQLSSLTSEDSAVYFCARWGYWGQGTLVT

SEQ ID NO: 105 - protein, 879 aa, Homo sapiens
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSAGSGGSGGSGAQVQLQESGGGLVQPGGSLRLSCAASGSISSITTMGWYRQDPGKGRELVALINSVGDTTYAGSGRFTISRDNAKNTVYLEMSSLKPEDTAVYYCNAFMSTNSGRTGSFWGQGTQVTVSSHHHHHHAGSGGSGGSGAEAAAKEAAAKEAAAKEAAAKEAAAKEAAAKSGSKAAAEKAAAEKAAAEKAAAEKAAAEKAAAEAGSGGSGGSGAQVQLQESGGGLVQPGGSLRLSCAASGSISSITTMGWYRQDPGKGRELVALINSVGDTTYAGSGRFTISRDNAKNTVYLEMSSLKPEDTAVYYCNAFMSTNSGRTGSFWGQGTQVTVSSHHHHHHSEKIHSNEISSDSIGVLGYQKTVDHTKVNSKLSLFFEIKS

SEQ ID NO: 106 - protein, 5 aa, artificial sequence
  synthetic polypeptide
EAAAK

SEQ ID NO: 107 - protein, 5 aa, artificial sequence
  synthetic polypeptide
KAAAE

SEQ ID NO: 108 - protein, 6 aa, artificial sequence
  synthetic polypeptide
EAAAAK

SEQ ID NO: 109 - protein, 6 aa, artificial sequence
  synthetic polypeptide
KAAAAE

SEQ ID NO: 110 - protein, 7 aa, artificial sequence
  synthetic polypeptide
EAAAAAK

SEQ ID NO: 111 - protein, 7 aa, artificial sequence
  synthetic polypeptide
KAAAAAE

SEQ ID NO: 112 - protein, 8 aa, artificial sequence
  synthetic polypeptide
EAAKAAKA

SEQ ID NO: 113 - protein, 8 aa, artificial sequence
  synthetic polypeptide
AKAAKAAE

SEQ ID NO: 114 - protein, 5 aa, artificial sequence
  synthetic polypeptide
  MISC_FEATURE (2)..(4): Amino acids at positions 2 to 4 may be repeated 1, 2, 3, 4, 5 times or more.
AGSGA

SEQ ID NO: 115 - protein, 11 aa, artificial sequence
  synthetic polypeptide
AGSGGSGGSGA

SEQ ID NO: 116 - protein, 164 aa, artificial sequence
  synthetic polypeptide
GENITQPTQQSTQATQRFLIEKFSQEQIGENIVCRVICTTGQIPIRDLSADISQVLKEKRSIKKVWTFGRNPACDYHLGNISRLSNKHFQILLGEDGNLLLNDISTNGTWLNGQKVEKNSNQLLSQGDEITVGVGVESDILSLVIFINDKFKQCLEQNKVDRIR

SEQ ID NO: 117 - protein, 11 aa, artificial sequence
  synthetic polypeptide
AGSGGSGGSGA

SEQ ID NO: 118 - protein, 229 aa, artificial sequence
  synthetic polypeptide
MSKGEELFTGVVPILVELDGDVNGHKFSVSGEGEGDATYGKLTLKFICTTGKLPVPWPTLVTTLTWGVQCFSRYPDHMKRHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGI

SEQ ID NO: 119 - protein, 65 aa, artificial sequence
  synthetic polypeptide
EAAAKEAAAKEAAAKEAAAKEAAAKEAAAKAGSGAKAAAEKAAAEKAAAEKAAAEKAAAEKAAAE

SEQ ID NO: 120 - protein, 229 aa, artificial sequence; identical to SEQ ID NO: 118
  synthetic polypeptide

SEQ ID NO: 121 - protein, 350 aa, artificial sequence
  synthetic polypeptide
GNAAAAKKGSEQESVKEFLAKAKEDFLKKWENPAQNTAHLDQFERIKTLGTGSFGRVMLVKHMETGNHYAMKILDKQKVVKLKQIEHTLNEKRILQAVNFPFLVKLEFSFKDNSNLYMVMEYVPGGEMFSHLRRIGRFSEPHARFYAAQIVLTFEYLHSLDLIYRDLKPENLLIDQQGYIQVTDFGFAKRVKGRTWTLCGTPEYLAPEIILSKGYNKAVDWWALGVLIYEMAAGYPPFFADQPIQIYEKIVSGKVRFPSHFSSDLKDLLRNLLQVDLTKRFGNLKNGVNDIKNHKWFATTDWIAIYQRKVEAPFIPKFKGPGDTSNFDDYEEEEIRVSINEKCGKEFSEF

SEQ ID NO: 122 - protein, 140 aa, artificial sequence
  synthetic polypeptide
  misc_feature (66)..(66): Xaa can be any naturally occurring amino acid
MSKGEELFTGVVPILVELDGDVNGHKFSVSGEGEGDATYGKLTLKFICTTGKLPVPWPTLVTTFGXGLQCFARYPDHMKRHDFFKSAMPEGYVQERTIFFKDDGNYKTRAEVKFEGDTLVNRIELKGIDFKEDGNILGHK

SEQ ID NO: 123 - protein, 98 aa, artificial sequence
  synthetic polypeptide
LEYNYNSQNVYIMADKQKNGIKVNFKIRHNIEDGSVQLADHYQQNTPIGDGPVLLPDNHYLSYQSALSKDPNEKRDHMVLLEFVTAAGITHGMDELYK

SEQ ID NO: 124 - protein, 30 aa, artificial sequence
  synthetic polypeptide
SAGKPGSGEGSTKGLRRATVLDGGTGGSEL

SEQ ID NO: 125 - protein, 124 aa, artificial sequence
  synthetic polypeptide
QVQLQESGGGLVQPGGSLRLSCAASGSISSITTMGWYRQDPGKGRELVALINSVGDTTYAGSGRFTISRDNAKNTVYLEMSSLKPEDTAVYYCNAFMSTNSGRTGSFWGQGTQVTVSSHHHHHH

SEQ ID NO: 126 - protein, 240 aa, artificial sequence
  synthetic polypeptide
DYKDIVLTQSHKFMSTSVGDRVSITCKASQDVGTAVAWYQQKPGQSPKLLIYWASTRHTGVPDRFTGSGSGTDFTLTISNVQSEDLADYFCQQYSSYPLTFGAGTKLELKRGGGGSGGGGSGGGGSGGGGSQVQLQEPGGELVRPGASVKLSCKASGYTFTSYWINWVKQRPGQGLEWIGNIYPSDSYTNYNQKFKDKATLTVDKSSSTAYMQLSSLTSEDSAVYFCARWGYWGQGTLVT

SEQ ID NO: 127 - protein, 495 aa, artificial sequence
  synthetic polypeptide
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSS

SEQ ID NO: 128 - protein, 40 aa, artificial sequence
  synthetic polypeptide
SEKIHSNEISSDSIGVLGYQKTVDHTKVNSKLSLFFEIKS